(19) United States
(12) Patent Application Publication
(10) Pub. No.: US 2024/0100150 A1
(43) Pub. Date: Mar. 28, 2024

(54) REVERSE GENETICS-BASED COMPOSITIONS OF RECOMBINANT SARS-COV-2

(71) Applicants: TEXAS BIOMEDICAL RESEARCH INSTITUTE, San Antonio, TX (US); Spanish National Centre for Biotechnology, Madrid (ES); Scripps Research, La Jolla, CA (US)

(72) Inventors: Luis Martinez-Sobrido, San Antonio, TX (US); Chengjin Ye, San Antonio, TX (US); Fernando Almazan Toral, Madrid (ES); Juan Carlos de La Torre, La Jolla, CA (US)

(21) Appl. No.: 18/255,612

(22) PCT Filed: Dec. 2, 2021
(86) PCT No.: PCT/US21/72714

§ 371 Date: Jun. 2, 2023

Related U.S. Application Data
(60) Provisional application No. 63/203,914, filed on Aug. 4, 2021.
(60) Provisional application No. 63/199,021, filed on Dec. 2, 2020.

(51) Int. Cl. A61K 39/215; A61P 31/14; C12N 15/70

(52) U.S. Cl. CPC A61K 39/215; A61P 31/14; C12N 15/70; A61K 2039/53; C12N 2770/20022; C12N 2770/20034; C12N 2770/20062; C12N 2770/20071; C12N 2800/204

(57) ABSTRACT

Provided here are compositions for a bacterial artificial chromosome-based construct containing a replication-competent recombinant severe acute respiratory syndrome coronavirus-2 genome and methods of making such compositions and uses thereof.

### CROSS-REFERENCE TO RELATED APPLICATIONS

This present patent application claims priority to, and the benefit of, the pending U.S. Provisional Patent Application No. 63/199,021, filed Dec. 2, 2020, and U.S. Provisional Patent Application No. 63/203,914, filed Aug. 4, 2021, which are incorporated by reference herein in their entirety.

### TECHNICAL FIELD

The present disclosure relates to recombinant bacterial artificial chromosome (BAC) constructs containing coronavirus polynucleotides, methods of making such compositions, and methods of use of such compositions.

### BACKGROUND

The pandemic coronavirus (CoV) disease 2019 (COVID-19) caused by severe acute respiratory syndrome coronavirus-2 (SARS-CoV-2) is a major threat to global human health. As of November 2021, SARS-CoV-2 has spread worldwide and it has been responsible of over 250 million confirmed cases and around 5 million deaths. SARS-CoV-2 is a single-stranded, positive-sense RNA Betacoronavirus that belongs to the Coronaviridae family. Prior to SARS-CoV-2, only six coronavirus (CoVs) species were known to cause disease in humans. Of the six, four human (h)CoVs are prevalent and responsible of causing common cold in immunocompetent individuals (hCoV-229E, hCoV-OC43, hCoV-NL63, and hCoV-HKU1). The two other CoVs, severe acute respiratory syndrome coronavirus (SARS-CoV) and Middle East respiratory syndrome coronavirus (MERS-CoV), have been associated with severe illness and significant morbidity and mortality. SARS-CoV was responsible for an outbreak of severe acute respiratory syndrome in 2002-2003 in Guangdong Province, China, with a fatality rate of around 9.5%. MERS-CoV was responsible for an outbreak of severe respiratory disease in 2012-2013 in the Middle East, with a fatality rate of around 30%. SARS-CoV-2 has a viral genome of approximately 30,000 nucleotides in length and high similarity to that of SARS-CoV (˜79%) and lower to MERS-CoV (˜50%). The fatality rate of SARS-CoV-2 can be as high as 49% in critically ill patients making the COVID-19 pandemic rival that of the “Spanish flu” in 1918-1919.

### SUMMARY

There is an urgent need to identify effective prophylactics and therapeutics for the treatment of SARS-CoV-2 infection and associated COVID-19 disease. Recombinant BAC constructs containing the recombinant SARS-CoV-2 (rSARS-CoV-2) have been developed for use in screening of antiviral agents and development of vaccines. These rSARS-CoV-2 compositions possesses the same phenotype as the natural isolate in vitro and in vivo in two validated rodent (K18 hACE2 transgenic mice and golden Syrian hamsters) animal models of SARS-CoV-2 infection.

Embodiments include a bacterial artificial chromosome-based construct containing a replication-competent recombinant SARS-CoV-2 genome, USA-WA1/2020 strain. The rSARS-CoV-2 genome contains one or more of the following: a portion of or the complete SARS-CoV-2 genome, a deletion of a group-specific open reading frame, or a reporter, such as a fluorescent or luciferase gene adapted to report transcription of the rSARS-CoV-2 genome. The group-specific open reading frame can be one or more of Spike (S), Envelope (E), ORF3a, ORF6, ORF7a, ORF7B, ORF8, and ORF10. In an embodiment, the reporter gene encodes a fluorescent protein. The fluorescent protein can be a red fluorescent protein or a yellow fluorescent protein. In an embodiment, the reporter gene encodes a luciferase. In an embodiment, the compositions are used in immunization of a subject.

The replication-competent rSARS-CoV-2 compositions expressing fluorescent (such as, Venus or mCherry), or bioluminescent (such as, Nluc) reporter genes were generated using the bacterial artificial chromosome (BAC)-based reverse genetics methods described herein. These recombinant compositions were used in methods to track viral infections in vitro. These reporter-expressing rSARS-CoV-2 display similar growth kinetics and plaque phenotype as their wild-type counterpart (rSARS-CoV-2/WT). These compositions can be used in methods to identify chemical agents or neutralizing antibodies for the therapeutic treatment of SARS-CoV-2. These reporter-expressing rSARS-CoV-2 compositions can be used to interrogate large libraries of compounds or antibodies, in high-throughput screening settings, to identify those with therapeutic potential against SARS-CoV-2. These compositions provide a direct correlation between reporter gene expression and viral replication, and infected cells can be easily detected, without the need of secondary approaches, based on reporter gene expression.

Embodiments also include a reverse genetics system for screening and identifying an anti-SARS-CoV-2 agent. One such system includes a bacterial artificial chromosome-based construct containing a replication-competent SARS-CoV-2 genome. In an embodiment, the SARS-CoV-2 genome contains a deletion of a group-specific open reading frame and a reporter gene adapted to report transcription of the SARS-CoV-2 genome. Embodiments also include a bacterial artificial chromosome-based construct containing a replication-competent recombinant SARS-CoV-2 genome. In an embodiment, the SARS-CoV-2 genome contains a mutation in the gene encoding for a spike protein and a reporter gene adapted to report transcription of the SARS-CoV-2 genome. In an embodiment, the mutation is a Bristol deletion or a Furin deletion. Using these reporter-expressing rSARS-CoV-2 compositions, fluorescent-based microneutralization assays have been developed that can be used to identify neutralizing antibodies (NAbs) or antiviral agents. The neutralization titers and inhibitory activities of NAbs or antivirals, respectively, obtained in these reporter-based microneutralization assays were similar to those observed in classical microneutralization assays using rSARS-CoV-2/WT. These reporter-expressing rSARS-CoV-2 compositions facilitate characterization of the virus and methods for the identification of therapeutics for the treatment of SARS-CoV-2. These rSARS-CoV-2 compositions expressing foreign genes can be used to generate vaccines for the treatment of SARS-CoV-2 infections and/or associated COVID-19 disease.

Disclosed here are vaccine compositions against SARS-CoV-2 and methods of generation, safety testing, and protection efficacy of the live attenuated vaccines against SARS-CoV-2. Using the reverse genetics system, recombinant (r) SARS-CoV-2 with double open reading frame (ORF) deletions, rSARS-CoV-2 Δ3a/Δ6, Δ3a/Δ7a, and Δ3a/Δ7b were rescued and evaluated for safety, protection, and immunogenicity. When characterized in vitro, all double ORF mutants were significantly attenuated in both VeroE6 and Δ549 cell lines expressing the human angiotensin converting enzyme 2 (hACE2) and produced smaller plaque morphologies. When tested for virulence and pathogenicity in the K18 hACE-2 transgenic SARS-CoV-2 mouse animal model, rSARS-CoV-2 Δ3a/Δ7a was lethal in 25% of animals, while animals challenged with rSARS-CoV-2 Δ3a/Δ6 and Δ3a/Δ7b had a 100% survival rate. The protection efficacy of rSARS-CoV-2 Δ3a/Δ6 or rSARS-CoV-2 Δ3a/Δ7b in vaccinated animals was further evaluated by a challenge with a lethal dose of rSARS-CoV-2 Nluc-2A. Both vaccine compositions were able to provide protection and resulted in delayed and controlled immune response. Both rSARS-CoV-2 Δ3a/Δ6 or rSARS-CoV-2 Δ3a/Δ7b can serve as vaccine candidates against SARS-CoV-2.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. To facilitate this description, like reference numerals designate like structural elements or procedures in a method. Embodiments are illustrated by way of example and not by way of limitation in the figures of the accompanying drawings.

FIG. 1A is a schematic representation of the SARS-CoV-2 genome, USA-WA1/2020 strain. Length is not to scale. FIG. 1B is a schematic representation of the full-length infectious cDNA clone as assembled by sequentially cloning chemically synthesized fragments 1 to 5, which cover the entire viral genome, into the pBeloBAC11 plasmid by using the indicated restriction sites under the control of the cytomegalovirus (CMV) promoter. The clone was flanked at the 3′ end by the hepatitis delta virus (HDV) ribozyme (Rz) and the bovine growth hormone (bGH) termination and polyadenylation sequences. FIG. 1C is a photographic representation of the analysis of the BAC clone harboring the entire viral genome after digestion with the indicated restriction enzymes (top), as analyzed in a 0.5% agarose gel.

FIG. 2A is a schematic representation of the method used to generate rSARS-CoV-2 compositions. FIG. 2B is a set of photographic images of empty BAC-transfected (left panel) or SARS-CoV-2 BAC-transfected (right panel) Vero E6 cells at 72 h post transfection to evaluate the cytopathic effect (CPE). Scale bars, 100 m. FIG. 2C is a graphical representation of the viral titers in these cell lines. Data are presented as means SDs. LOD, limit of detection. FIG. 2D is a set of photographic images following evaluation of empty BAC (left panel) or SARS-CoV-2 BAC (right panel) infected Vero cells as analyzed by an immunofluorescence assay. N protein (green), 4,6-diamidino-2-phenylindole (DAPI; blue). Scale bars, 100 m.

FIG. 3A is a photographic representation of undigested (top panel) and digested (bottom panel) samples of the rSARS-CoV-2 or the SARS-CoV-2 constructs as analyzed on an agarose gel. FIG. 3B is an illustration of the MluI restriction site (underlined in red) in the SARS-CoV-2 construct (bottom panel), and the silent mutation introduced in the rSARS-CoV-2 construct (top panel) to remove the MluI restriction site (T to A) is shown in the black box. FIG. 3C is a representation of verification of SARS-CoV-2 sequence (top panel), rSARS-CoV-2 sequence (middle panel), and SARS-CoV-2 BAC sequence (bottom panel). Non-reference alleles present in less than 1% of reads are not shown. FIG. 3D is a photographic representation of the plaque phenotype following infection of Vero E6 cells with 20 PFU of rSARS-CoV-2 (left) or the natural SARS-CoV-2 isolate (right) and immunostaining analysis with the N protein 1C7 monoclonal antibody. FIG. 3E is a graphical representation of the growth kinetics of Vero E6 cells infected (MOI, 0.01) with rSARS-CoV-2 or the natural SARS-CoV-2 isolate. Data are presented as means SDs. LOD, limit of detection.

FIG. 4A is a set of photographs of gross pathological lung lesions to demonstrate the pathogenicity of rescued rSARS-CoV-2 in vivo. Golden Syrian hamsters were mock infected (n2) or infected (n6) with 2104 PFU of rSARS-CoV-2 or SARS-CoV-2. Animals were euthanized at 2 and 4 days' post infection, and lungs from mock-infected (i and iv) or infected (rSARS-CoV-2 [ii and v] and SARS-CoV-2 [iii and vi]) animals were observed for gross pathological changes, including congestion and atelectasis (white arrows) and frothy trachea exudates (black arrows). Scale bars, 1 cm. FIG. 4B is a graphical representation of the macroscopic pathology scoring analysis of lungs from rSARS-CoV-2 and SARS-CoV-2 animals at Day 2 and Day 4. Distributions of pathological lesions, including consolidation, congestion, and atelectasis, were measured using ImageJ and are represented as percentages of the total lung surface area. ns, not significant. FIG. 4C and FIG. 4D are graphical representations of the viral titers in the lungs and nasal turbinates, respectively, of rSARS-CoV-2- and SARS-CoV-2-infected golden Syrian hamsters as evaluated at days 2 and 4 post infection (3 hamsters per time point). Data are means SDs. ns, not significant.

FIG. 5A is a schematic representation of the rSARS-CoV-2 constructs with Venus, mCherry, or Nluc genes. FIG. 5B is a photographic image of the RT-PCR analysis of the rSARS-CoV-2 constructs containing Venus, mCherry, or Nluc reporters, for the presence of the viral NP, the ORF7a region, or the individual reporter genes (Venus, mCherry, or Nluc).

FIG. 6A is a set of photographic images under fluorescence microscopy of cell cultures infected with a mock construct, a reporter-expressing rSARS-CoV-2 construct, and a rSARS-CoV-2/WT construct and evaluated for expression of Venus and mCherry and the SARS-CoV NP. FIG. 6B is a graphical representation of the expression of Nluc in rSARS-CoV-2-Nluc-infected cells and rSARS-CoV-2/WT infected cells at 48 h post-infection. FIG. 6C is a set of photographic images of Western blot assays using cell lysates from either mock, rSARS-CoV-2-WT, or rSARS-CoV-2-Venus (left panel), -mCherry (middle panel), or -Nluc (right panel) infected cells using monoclonal antibodies (MAbs) against the viral NP, the reporter genes, or actin as a loading control.

FIG. 7A is a set of photographic images under bright-field and fluorescence microscopy of cell cultures infected with reporter-expressing rSARS-CoV-2 and rSARS-CoV-2/WT and evaluated for expression of control (left panel), Venus (middle panel) and mCherry (right panel) over a period of 96 hours. FIG. 7B is a graphical representation of the expression of Nluc in rSARS-CoV-2-Nluc-infected cells and rSARS-CoV-2/WT infected cells over a period of 96 hours as detected using a luminometer. FIG. 7C is a graphical representation of the growth kinetics of reporter-expressing rSARS-CoV-2 constructs to that of rSARS-CoV-2/WT. FIG. 7D is a set of photographic representations of the plaque phenotype following infection of Vero E6 cells with rSARS-CoV-2/WT (left panel), and rSARS-CoV-2 expressing fluorescent reporter genes-rSARS-CoV-2-Venus (middle panel) and rSARS-CoV-2-mCherry (right panel) as analyzed by fluorescence and immunostaining.

FIG. 8A is a graphical representation of the EC₅₀ of Remdesivir against a rSARS-CoV-2-Venus construct in a reporter-based microneutralization assay. FIG. 8B is a photographic image of the immunohistochemical analysis of the reporter-expressing rSARS-CoV-2 assay for rSARS-CoV-2-Venus construct under different concentrations of Remdesivir. FIG. 8C is a graphical representation of the EC₅₀ of Remdesivir against a rSARS-CoV-2-mCherry construct in a reporter-based microneutralization assay. FIG. 8D is a photographic image of the immunohistochemical analysis of the reporter-expressing rSARS-CoV-2 assays for rSARS-CoV-2-mCherry under different concentrations of Remdesivir. FIG. 8E is a graphical representation of the EC₅₀ of Remdesivir against a rSARS-CoV-2-Nluc construct in a reporter-based microneutralization assay. FIG. 8F is a graphical representation of the EC₅₀ of Remdesivir against a rSARS-CoV-2/WT construct in a reporter-based microneutralization assay.

FIG. 9A is a graphical representation of the NT₅₀ of 1212C2 against a rSARS-CoV-2-Venus construct in a reporter-based microneutralization assay. FIG. 9B is a photographic image of the immunohistochemical analysis of the reporter-expressing rSARS-CoV-2 assay for rSARS-CoV-2-Venus construct under different concentrations of 1212C2. FIG. 9C is a graphical representation of the NT₅₀ of 1212C2 against a rSARS-CoV-2-mCherry construct in a reporter-based microneutralization assay. FIG. 9D is a photographic image of the immunohistochemical analysis of the reporter-expressing rSARS-CoV-2 assays for rSARS-CoV-2-mCherry under different concentrations of 1212C2. FIG. 9E is a graphical representation of the NT₅₀ of 1212C2 against a rSARS-CoV-2-Nluc construct in a reporter-based microneutralization assay. FIG. 9F is a graphical representation of the NT₅₀ of 1212C2 against a rSARS-CoV-2/WT construct in a reporter-based microneutralization assay.

FIG. 10A is a set of photographic images of the Venus (top panels) and mCherry (bottom panels) fluorescent expression from the rSARS-CoV-2 constructs as evaluated before immunostaining with an anti-SARS-CoV NP MAb 1C7 at a particular passage (P3, right panels) with those of additional passages (P4, middle panels and P5, left panels). FIG. 10B is a set of analysis of genome sequences of the reporter-expressing rSARS-CoV-2 constructs—Venus (top panels), mCherry (middle panels), and Nluc (bottom panels)—used herein at a particular passage (P3, right panels) with those of additional passages (P4, middle panels and P5, left panels) using NGS.

FIG. 11A is a diagrammatic representation of the SARS-CoV-2/WT and the various Venus, mCherry, or Nluc expressing rSARS-CoV-2 compositions. FIG. 11B is a diagrammatic representation of the various rSARS-CoV-2 compositions that are deficient in specific viral genes. FIG. 11C is a diagrammatic representation of the various rSARS-CoV-2 compositions that contain mutations in the Spike protein.

FIG. 12A is a diagrammatic representation of the rSARS-CoV-2 reporter viruses with the Venus or mCherry fluorescent proteins (FP). FIGS. 12B and 12C are sets of photographic images of Vero E6 cells infected with either rSARS-CoV-2 WT, rSARS-CoV-2 Venus, rSARS-CoV-2 mCherry, or mock-infected, and then visualized by fluorescence microscopy. FIGS. 12D and 12E are graphical representations of the multi-step growth kinetics (viral titers and Fluorescent PFUs) of the rSARS-CoV-2 Venus or rSARS-CoV-2 mCherry, individually or together in tissue culture supernatants collected over a course of 96 hours. FIG. 12F is a set of photographic images of Vero E6 cells expressing rSARS-CoV-2 Venus or rSARS-CoV-2 mCherry, individually and in combination, and then visualized by fluorescence microscopy at four time points (24, 48, 72, and 96 hours). FIG. 12G is a set of photographic images of plaque formation of rSARS-CoV-2 WT, rSARS-CoV-2 Venus, rSARS-CoV-2 mCherry, individually and in combination.

FIGS. 13A-13F are sets of graphical representations and photographic images directed to the bifluorescent-based assay to identify Nabs. Confluent monolayers of Vero E6 cells (4×10⁴ cells/well, 96-plate well format, quadruplicates) were infected (MOI 0.1) with rSARS-CoV-2 Venus (FIGS. 13A and 13D), rSARS-CoV-2 mCherry (FIGS. 13B and 13E), or both rSARS-CoV-2 Venus and rSARS-CoV-2 mCherry (FIGS. 13C and 13F), respectively. After 1 h infection, post-infection (pi) media containing 3-fold serial dilutions of 1212C2 (FIGS. 13A-13C) or 1213H7 (FIGS. 13D-13F) hMAbs (starting concentration at 500 ng) was added to the cells. At 48 hours post-infection (hpi), cells were fixed with 10% neutral buffered formalin and levels of fluorescence expression were quantified in a fluorescent plate reader and analyzed using Gen5 data analysis software (BioTek). NT₅₀ values of 1212C2 and 1213H7 hMAbs for each virus, alone or in combination, were determined using GraphPad Prism. Dashed lines indicate 50% viral neutralization. Data are means and SD from quadruplicate wells. Representative images are shown. Scale bars=300 μm.

FIGS. 14A-14G are directed to the generation and characterization of rSARS-CoV-2 mCherry SA. FIG. 14A is a schematic representation of rSARS-CoV-2 mCherry SA construct. The genome of a rSARS-CoV-2 Venus (top) and the rSARS-CoV-2 with the three mutations (K417N, E484K, and N501Y) present in the S RBD of the SA B.1.351 (beta, f) VoC expressing mCherry (bottom) is shown. FIG. 14B is a set of images from the sequencing of rSARS-CoV-2 mCherry SA. Sanger sequencing results of the rSARS-CoV-2 Venus (top panels) and the rSARS-CoV-2 mCherry SA with the K417N (left), E484K (middle), and N501Y (right) substitutions in the RBD of the S glycoprotein (bottom panels) are indicated. FIG. 14C is a set of photographic images of reporter gene expression. Vero E6 cells (6-well plate format, 10⁶ cells/well, triplicates) were mock-infected or infected (MOI 0.01) with rSARS-CoV-2, rSARS-CoV-2 Venus, or rSARS-CoV-2 mCherry SA. Infected cells were fixed in 10% neutral buffered formalin at 24 hpi and visualized under a fluorescence microscope for Venus or mCherry expression. FIGS. 14D and 14E are graphical representations of the multi-step growth kinetics (viral titers and Fluorescent PFUs) of the rSARS-CoV-2 Venus or rSARS-CoV-2 mCherry SA, individually or together in tissue culture supernatants collected over a course of 96 hours. FIG. 14F is the corresponding set of photographic images. Vero E6 cells (6-well plate format, 10⁶ cells/well, triplicates) were mock-infected or infected (MOI 0.01) with rSARS-CoV-2 Venus, rSARS-CoV-2 mCherry SA, or both rSARS-CoV-2 Venus and rSARS-COV-2 mCherry SA. Tissue cultured supernatants were collected at the indicated times p.i. to assess viral titers using standard plaque assay (FIG. 14D). The amount of Venus- and/or mCherry-positive plaques at the same times p.i. were determined using fluorescent microscopy (FIG. 14E). Images of infected cells under a fluorescent microscope at the same times p.i. are shown (FIG. 14F). FIG. 14G is a set of photographic images of the plaque assay. Vero E6 cells (6-well plate format, 10⁶ cells/well, triplicates) were mock-infected or infected with ˜20 PFU of rSARS-CoV-2, rSARS-CoV-2 Venus, rSARS-CoV-2 mCherry SA, or both rSARS-CoV-2 Venus and rSARS-CoV-2 mCherry SA. At 72 hpi, fluorescent plaques were assessed using a Chemidoc instrument. Viral plaques were also immunostained with the SARS-CoV N protein 1C7C7 cross-reactive mMAb. Fluorescent green, red and merge imaged as shown. Representative images are shown for panels C, F and G. Scale bars=300 μm.

FIGS. 15A-15F are sets of graphical representations and photographic images directed to a bifluorescent-based assay to identify SARS-CoV-2 broadly Nabs. Confluent monolayers of Vero E6 cells (4×10⁴ cells/well, 96-plate well format, quadruplicates) were infected (MOI 0.1) with rSARS-CoV-2 Venus (FIGS. 15A and 15D), rSARS-CoV-2 mCherry SA (MOI 0.01) (FIGS. 15B and 15E), o both rSARS-CoV-2 Venus (MOI 0.1) and rSARS-CoV-2 mCherry SA (MOI 0.01) (FIGS. 15C and 15F). After 1 h infection, p.i. media containing 3-fold serial dilutions of 12C2C2 (FIGS. 15A-15C) or 1213H7 (FIGS. 15D-15F) hMAbs (starting concentration of 500 ng) was added to the cells. At 48 hpi, cells were fixed with 10% neutral buffered formalin and levels of fluorescence expression were quantified in a fluorescent plate reader and analyzed using Gen5 data analysis software (BioTek). The NT₅₀ values of 1212C2 and 1213H7 hMAbs for each virus, alone or in combination, were determined using GraphPad Prism. Dashed lines indicate 50% viral neutralization. Data are means and SD from quadruplicate wells. Representative images are shown. Scale bars=300 μm.

FIGS. 16A-16I are graphical representations directed to bifluorescent-based assays for identification of SARS-CoV-2 broadly NAbs. Confluent monolayers of Vero E6 cells (4×10⁴ cells/well, 96-plate well format, quadruplicates) were co-infected with rSARS-CoV-2 Venus (MOI 0.1) and rSARS-CoV-2 mCherry SA (MOI 0.01). After 1 h infection, p.i. media containing 3-fold serial dilutions (starting concentration 500 ng) of the indicated hMAbs was added to the cells. At 48 hpi, cells were fixed with 10% neutral buffered formalin and levels of fluorescence were quantified using a fluorescent plate reader and analyzed using Gen5 data analysis software (BioTek). NT₅₀ values of each of the hMAbs was determined using GraphPad Prism. Dashed lines indicate 50% viral neutralization. FIGS. 16A, 16B, 16C, 16D, 16E, 16F, 16G, 16H, and 16I are results for the treatment of the cells with CB6, REGN10933, REGN10987, 1206D12, 1212D5, hMAb 1215D1, 1206G12, 1212F2, and 1207B4 antibodies, respectively. Data are means and SD from quadruplicate wells. Representative images are shown. Scale bars=300 μm.

FIGS. 17A and 17B are sets of graphical representations of body weight (17A) and survival (17B) of K18 hACE2 transgenic mice treated with 1212C2 and 1213H7 against rSARS-CoV-2 Venus and rSARS-CoV-2 mCherry SA, alone or in combination. Six-to-eight-week-old female K18 hACE2 transgenic mice (n=5) were treated (i.p.) with 25 mg/kg of IgG isotype control, hMAb 1212C2, or hMAb 1213H7 and infected with 10⁴ PFU of rSARS-CoV-2 Venus (left), rSARS-CoV-2 mCherry SA (middle) or both, rSARS-CoV-2 Venus and rSARS-CoV-2 mCherry SA (right). Mice were monitored for 12 days for changes in body weight (17A) and survival (17B). Data represent the means and SD of the results determined for individual mice.

FIGS. 18A and 18B are sets of photographic images and graphical representations of the kinetics of fluorescent expression in the lungs of K18 hACE2 transgenic mice treated with 1212C2 or 1213H7 hMAbs and infected with rSARS-CoV-2 Venus and rSARS-CoV-2 mCherry SA. Six-to-eight-week-old female K18 hACE2 transgenic mice (n=3) were injected (i.p.) with 25 mg/kg of an IgG isotype control, hMAb 1212C2, or hMAb 1213H7 and infected with 10⁴ PFU of rSARS-CoV-2 Venus (top), rSARS-CoV-2 mCherry SA (middle), or both, rSARS-CoV-2 Venus and rSARS-CoV-2 mCherry SA (bottom). At days 2 and 4 pi, lungs were collected to determine Venus and mCherry fluorescence expression using an Ami HT imaging system (FIG. 18A). Venus and mCherry radiance values were quantified based on the mean values for the regions of interest in mouse lungs (FIG. 18B). Mean values were normalized to the autofluorescence in mock-infected mice at each time point and were used to calculate fold induction. Gross pathological scores in the lungs of mock-infected and rSARS-CoV-2-infected K18 hACE2 transgenic mice were calculated based on the % area of the lungs affected by infection. BF, bright field.

FIGS. 19A-19D are sets of graphical representations of viral titers in the lungs, nasal turbinate and brain, respectively, of K18 hACE2 transgenic mice treated with 1212C2 or 1213H7 hMAbs and infected with rSARS-CoV-2 Venus and rSARS-CoV-2 mCherry SA. Six-to-eight-week-old female K18 hACE2 transgenic mice (n=3) injected (i.p.) with 25 mg/kg of an IgG isotype control, hMAb 1212C2, or hMAb 1213H7 and infected with 10⁴ PFU of rSARS-CoV-2 Venus (A), rSARS-CoV-2 mCherry SA (B) or both, rSARS-CoV-2 Venus and rSARS-CoV-2 mCherry SA (C). Viral titers in the lungs (top), nasal turbinate (middle) and brain (bottom) at days 2 and 4 p.i. were determined by plaque assay in Vero E6 cells. Bars indicates the mean and SD of lung virus titers. Dotted lines indicate the limit of detection. FIG. 19D is a graphical representation of the quantification of rSARS-CoV-2 Venus and rSARS-CoV-2 mCherry SA in the lungs (top), nasal turbinate (middle) and brain (bottom) from mice infected with both viruses at days 2 and 4 pi.

FIG. 20 is a set of diagrammatic representations of the various genome organizations of the WT and ΔORF rSARS-CoV-2s. The SARS-CoV-2 genome includes 29.8 kb of nucleotides, among which 21.5 kb encodes the ORF1a and ORF1b replicase. The rest of the 8.3-kb viral genome encodes the structural spike (S), envelope (E), matrix (M), and nucleocapsid (N) proteins and the accessory ORF3a, 26, 27a, 27b, 28, and 210 proteins. Individual deletions of the ORF accessory proteins were introduced into the BAC for rescue of rSARS-CoV-2. Schematic representations are not drawn to scale.

FIG. 21A is a set of photographic images obtained from immunofluorescence assay using WT or the ORFΔ3a, ORFΔ6, ORFΔ7a, ORFΔ7b, or ORFΔ8 rSARS-CoV-2. Vero E6 cells (24-well plate format, 10⁵ cells/well, triplicates) were mock infected or infected (MOI of 3) with the WT or the ORFΔ3a, ORFΔ6, ORFΔ7a, ORFΔ7b, or ORFΔ8 rSARS-CoV-2. At 24 h p.i., cells were fixed and immunostained with a cross-reactive polyclonal antibody against SARS-CoV N protein and a cross-reactive monoclonal antibody against SARS-CoV S protein (3B4). Rhodamine red goat anti-rabbit and FITC rabbit anti-mouse secondary antibodies were used, and nuclei were visualized with DAPI. Scale bars, 100 mm. Vero E6 cells (6-well plate format, 10⁶ cells/well) were mock infected or infected (MOI of 0.1) with the WT or a ΔORF rSARS-CoV-2, and total RNA was extracted at 24 h p.i. Regions in the viral genome corresponding with the deletion were amplified, and the N gene was amplified as an internal control. FIG. 21B is a set of photographic images of the agarose gel separation of these amplified RT-PCR products. MW, molecular weight. FIG. 21C is a set of images from the sequencing of the RT-PCR products from FIG. 21B, which were gel purified and subjected to Sanger sequencing. The consensus sequences in the genomes of both the WT and a ΔORF rSARS-CoV-2 downstream of the deleted gene are indicated in blue, the intergenic regions between viral genes are shown in yellow, the genes deleted from the ΔORF rSARS-CoV-2s are indicated in green, and the viral genes upstream of the deleted ORFs in the rSARS-CoV-2s are shown in red.

FIGS. 22A-22E are graphical representations from deep sequencing analysis of ORF-deficient rSARS-CoV-2s—rSARS-CoV-2/Δ3a, rSARS-CoV-2/Δ6, rSARS-CoV-2/Δ7a, rSARS-CoV-2/Δ7b, and rSARS-CoV-2/Δ8, respectively. The ORF-deficient rSARS-CoV-2 non-reference allele frequency was calculated by comparing short reads to the sequence of the respective reference WT SARS-CoV-2 USA-WA1/2020 strain genome. Silent mutations at positions 21895 and 26843 (according to the genome positions of the USA-WA1/2020 strain) were fixed in all ORF-deficient rSARS-CoV-2 genomes.

FIGS. 23A-23E are photographic images and graphical representations directed to the in vitro characterization of the WT and ΔORF rSARS-CoV-2s. FIG. 23A is a set of photographic images of the plaque phenotype from Vero E6 cells (6-well plate format, 10⁶ cells/well) infected with the WT, ORFΔ3a, ORFΔ6, ORFΔ7a, ORFΔ7b, or ORFΔ8 rSARS-CoV-2 and overlaid with medium containing agar. Plates were incubated at 37° C., and monolayers were immunostained with an anti-N protein SARS-CoV cross-reactive monoclonal antibody, 1C7C7, at the indicated hours p.i. FIG. 23B is a graphical representation of the viral plaque sizes using the WT and ΔORF rSARS-CoV-2s. Diameters of viral plaques were measured with a ruler in centimeters. Plaques less than 0.1 cm are indicated as not detected (ND). FIGS. 23C, 23D, and 23E are graphical representations of the multicycle growth kinetics in Vero E6 cells (FIG. 23C), hACE2-HEK293T cells (FIG. 23D), and hACE2-A549 (FIG. 23E) cells (6-well plate format, 10⁶ cells/well, triplicates) infected (MOI 0.01) with the WT or a ΔORF rSARS-CoV-2 and incubated at 37° C. At the indicated hours p.i., tissue culture supernatants from infected cells were collected, viral titers were determined by plaque assay (PFU per milliliter), and cells were immunostained using the anti-N SARS-CoV cross-reactive monoclonal antibody 1C7C7. Data are the means 6 standard deviations (SDs) of the results determined from triplicate wells. Dotted black lines indicate the limit of detection (LOD; 100 PFU/ml). *, P, 0.05, using the Student t test. ns, not significant.

FIGS. 24A-24B are graphical representations of body weight (24A) and survival (25B) of K18 hACE2 transgenic mice infected with the WT or a ΔORF rSARS-CoV-2. Six- to 8-week-old K18 hACE2 transgenic female mice were mock (PBS) infected or infected (i.n.) with 10⁵ PFU of the WT or a ΔORF rSARS-CoV-2 (n=4/group). Body weight (24A) and survival (25B) were evaluated at the indicated days p.i. Mice that lost 25% of their initial body weight were humanely euthanized. Error bars represent the SDs of the mean for each group.

FIGS. 25A-25B are graphical representations of the titers of the WT and ΔORF rSARS-CoV-2s in nasal turbinate and lungs, respectively. Six- to 8-week-old K18 hACE2 transgenic female mice were mock (PBS) infected or infected (i.n.) with 10⁵ PFU of the WT or a ΔORF rSARS-CoV-2 (n=8/group). Mice were sacrificed at 2 (n=4/group) and 4 (n=4/group) days p.i., and viral titers in nasal turbinate and lung were determined by plaque assay (PFU per milliliter) and immunostaining using the cross-reactive SARS-CoV 1C7C7 N protein monoclonal antibody. Viral titers in the nasal turbinate (25A) and lungs (25B) are shown. Symbols represent data from individual mice and bars the geometric means of viral titers. Dotted lines indicate the LOD (10 PFU/ml). ND, not detected; @, not detected in 1 mouse; #, not detected in 2 mice: &, not detected in 3 mice. Negative results of the PBS-infected mice are not plotted.

FIG. 26A is a set of photographic images from the gross pathology analysis of lungs from K18 hACE2 transgenic mice infected with the WT or a ΔORF rSARS-CoV-2. Lungs from 6-to-8-week-old female K18 hACE2 transgenic mice (n=8/group) mock (PBS) infected or infected (i.n.) with 10⁵ PFU of the WT or a ΔORF rSARS-CoV-2 were harvested at 2 (n=4/group) or 4 (n=4/group) days p.i., imaged (26A), and scored for virally induced lesions. FIG. 26B is a graphical representation of the percentage of surface area affected by virally induced lesions as observed from the gross pathology analysis of lungs from K18 hACE2 transgenic mice infected with the WT or a ΔORF rSARS-CoV-2. The total lung surface area affected by virally induced lesions were determined. Scale bars, 3 cm.

FIG. 27A is a set of graphical representations of the cytokine and chemokine storms in the lungs of K18 hACE2 transgenic mice mock infected and infected with the WT or a ΔORF rSARS-CoV-2 were determined using an 8-plex panel mouse ProcartaPlex assay. FIG. 27B is a schematic representation of the IL-6/IL-10 ratio as a marker of the local cytokine storm induced by rSARS-CoV-2. A two-way ANOVA of K18 hACE2 transgenic mice mock infected or infected with the WT or an ORF-deficient rSARS-CoV-2 was carried out with 4 mice per time point, except for the mock-infected group (n=2).

FIG. 28A is a schematic representation of the rSARS-CoV-2 genomes. The viral spike (S), envelope (E), matrix (M), and nucleocapsid (N) structural proteins; and the accessory 1a, 1b 3a, 6, 7a, 7b, 8, and 10 open reading frame (ORF) proteins are indicated. Double ORF deletions were introduced into the BAC for rescue of rSARS-CoV-2. Schematic representation not drawn to scale. FIG. 28B is a set of photographic images of RT-PCR products of regions in the viral genome corresponding to the deletions in the viral genome as analyzed on a 0.8% agarose gel. The viral N gene was amplified as internal control. FIG. 28C is a set of representations from deep sequencing analysis of the double ORF deficient rSARS-CoV-2 rSARS-CoV-2 Δ3a/Δ6, rSARS-CoV-2 Δ3a/Δ7a, and rSARS-CoV-2 Δ3a/Δ7b.

FIG. 29A is a set of photographic images of the plaque assay for in vitro characterization of wild-type and double ΔORF rSARS-CoV-2. FIG. 29B is a set of graphical representations of viral plaque sizes of wild-type and double ΔORF rSARS-CoV-2 constructs. FIGS. 29C and 29D are graphical representations of the multicycle growth kinetics of the wild-type and double ΔORF rSARS-CoV-2 constructs in Vero E6 (29C) and hACE-2 Δ549 (29D) cells. Data represent the means+/−standard deviations (SDs) of the results determined in triplicate wells. Dotted black lines indicate the limit of detection (LOD, 100 PFU/ml). P<0.05: using the Student T test.

FIGS. 30A-30B are graphical representations of the body weight (A) and survival (B) of K18 hACE2 transgenic mice infected with double ΔORF rSARS-CoV-2. K18 hACE2 transgenic female 6-8-week-old mice (N=13/group) were mock (PBS)-infected or infected (i.n.) with 10⁵ PFU of WT (pink circles) or ORFΔ3a/Δ6 (red squares), ORFΔ3a/Δ7a (blue triangles), or ORFΔ3a/Δ7b (green diamonds) double ΔORF rSARS-CoV-2. Body weight (A) and survival (B) (n=5/group) were evaluated at the indicated d p.i. Error bars represent SDs of the mean for each group. Mice were sacrificed at 2 (n=4/group) and 4 (n=4/group) d p.i. FIGS. 30C-30D are graphical representations of the viral titers in nasal turbinate (C) and lungs (D) of K18 hACE2 transgenic mice infected with double ΔORF rSARS-CoV-2 as determined by plaque assay (PFU/ml) and immunostaining using the cross-reactive SARS-CoV 1C7C7 N protein monoclonal antibody. LOD, limit of detection (100 PFU/ml). One-way ANOVA with multiple comparisons.

FIG. 31A is a set of photographic images from the gross pathology analysis of lungs from K18 hACE2 transgenic mice infected with wild-type and double ΔORF rSARS-CoV-2 at 2 and 4 days p.i., respectively. FIG. 30B is a graphical representation of viral induced lung lesions from the gross pathology analysis of lungs from K18 hACE2 transgenic mice infected with wild-type and double ΔORF rSARS-CoV-2 at 2 and 4 days p.i. Calculation represents the total lung surface area affected by viral induced lesions. Scale bars, 3 cm.

FIGS. 32A-32F are graphical representations of the humoral induced by double ΔORF rSARS-CoV-2 responses against SARS-CoV-2 variants. K18 hACE2 transgenic female 6-8-week-old mice were mock (PBS)-infected or infected (i.n.) with 105 PFU of ORFΔ3a/Δ6, ORFΔ3a/Δ7a, or ORFΔ3a/Δ7b double ΔORF rSARS-CoV-2 (n=4/group). At 14 (A) and 21 (B) d p.i., mice were bled and sera were collected and evaluated individually for the presence of total antibodies by ELISA (A-B). As a positive control, the SARS-CoV-2 anti-N monoclonal antibody (1C7C7) was used. Serum collected at 21 d p.i. were evaluated for their capacity to neutralize SARS-CoV-2 WA-1/2020 (32C), α (32D), β (32E), and ε (32F) variants of concern. FIG. 32G is a tabular representation of the NT₅₀ values for each assay.

FIG. 33A is a graphical representation of the cytokine and chemokine levels induced by wild-type and double ΔORF rSARS-CoV-2 infection. Cytokine and chemokine levels (picograms/milliliter) were measured in lung homogenates of K18 hACE2 transgenic mice infected with WT or double ΔORF rSARS-CoV-2 mutants at 2 dpi and 4 dpi. FIG. 27B is a schematic representation of the IL-6/IL-10 ratio as a marker of the local cytokine storm induced by wild-type and double ΔORF rSARS-CoV-2. IL-6/IL-10 ratio is shown for each of the time points as a marker of the local cytokine storm. Data is represented as M±SD with n=4 mice per group and time point; one-way ANOVA with multiple comparisons: *p<0.05, **p<0.005, ***p<0.0005, ****p<0.00005.

FIGS. 34A-34D are graphical representations of the in vivo protection efficacy of double ΔORF rSARS-CoV-2. K18 hACE2 transgenic female 6-8-week-old mice were mock (PBS)-infected (pink circles) or infected (i.n.) with 105 PFU of Δ3a/Δ6 or rSARS-CoV-2 Δ3a/Δ7b rSARS-CoV-2 (n=5/group). Body weight (34A) and survival (34B) were evaluated at the indicated d p.i. (n=5/group). Error bars represent SDs of the mean for each group. At 21 d p.i. mice where challenged with 10⁵ PFU of rSARS-CoV-2 Nluc-2A and body weight (34C) and survival (34D) were evaluated for 10 days. Error bars represent SDs of the mean for each group.

FIG. 35A is a set of photographic images of the expression of rSARS-CoV-2 Nluc in mice injected with PBS or rSARS-CoV-2 Δ3a/Δ6 or rSARS-CoV-2 Δ3a/Δ7a. K18 hACE2 transgenic female 6-8-week-old mice (n=8/group) were mock (PBS)-infected or infected (i.n.) with 10⁵ PFU of Δ3a/Δ6 or rSARS-CoV-2 Δ3a/Δ7b rSARS-CoV-2. At 21 d.p.i., the mice were mock-challenged or challenged (i.n.) with 10⁵ PFU of rSARS-CoV-2 Nluc-2A. Mice (n=4/group) were anesthetized at 1, 2, 4 and 6 d p.i. and retro-orbitally injected with the Nluc substrate. Nluc expression was determined using an AmiHT system. FIG. 35B is a graphical representation of the expression of rSARS-CoV-2 Nluc in double ΔORF rSARS-CoV-2 vaccinated mice as analyzed by the Aura program. FIGS. 35C-35D are graphical representations of the viral titers in nasal turbinate (C) and lungs (D) of double ΔORF rSARS-CoV-2 vaccinated mice, as determined by plaque assay (PFU/ml) and immunostaining using the cross-reactive SARS-CoV 1C7C7 N protein monoclonal antibody. LOD, limit of detection (100 PFU/ml).

FIG. 36A is a graphical representation of the cytokine and chemokine levels of vaccinated mice after challenge with rSARS-CoV-2 Nluc-2A. Cytokine and chemokine levels were measured in lung homogenates of K18 hACE2 transgenic mice mock vaccinated (pink) or vaccinated with double ΔORF rSARS-CoV-2 mutants ORFΔ3a/Δ6 (red), or ORFΔ3a/Δ7b (green) mutants at 2 dpi (n=4/group) and 4 dpi (n=4/group). Mice were challenged with 10⁵ PFU of rSARS-CoV-2 Nluc-2A. FIG. 36B is a schematic representation of the IL-6/IL-10 ratio as a marker of the local cytokine storm in K18 hACE2 transgenic mice mock vaccinated or vaccinated with double ΔORF rSARS-CoV-2 mutants after challenge with rSARS-CoV-2 Nluc-2A. IL-6/IL-10 ratio is shown for each of the time points as a marker of the local cytokine storm. Data represent the means+/−SDs of the results for 4 individual mice. One-way ANOVA with multiple comparisons: ns, not significance, *p<0.05, **p<0.005, ***<0.0005.

### DETAILED DESCRIPTION

The present disclosure describes various embodiments related to recombinant bacterial artificial chromosome (BAC) vectors containing coronavirus polynucleotides, methods of making such compositions, and methods of use. In the following description, numerous details are set forth in order to provide a thorough understanding of the various embodiments. Before the present methods and compositions are described, it is to be understood that these embodiments are not limited to particular methods or compositions described, as such may, of course, vary. It is also to be understood that the terminology used herein is for the purpose of describing particular embodiments only, and is not intended to be limiting, as the scope of the present embodiments will be limited only by the appended claims. The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous.

As used herein, the terms “treatment,” “treating,” and “treat” refer to any indicia of success in the treatment or amelioration of an injury, disease, or condition, including any objective or subjective parameter such as abatement, remission, diminishing of symptoms or making the injury, disease, or condition more tolerable to the subject, slowing in the rate of degeneration or decline, making the final point of degeneration less debilitating, and/or improving a subject's physical or mental well-being. The terms “administer,” “administering,” and “administration” refer to introducing a compound, a composition, or an agent (e.g., a rSARS-CoV-2 construct) into a subject or subject, such as a human. As used herein, the terms encompass both direct administration, e.g., self-administration or administration to a subject by a medical professional, and indirect administration, such as the act of prescribing a compound, composition, or agent.

As used herein, the term “adjuvant” refers to a compound that, when used in combination with an immunogen, augments or otherwise alters or modifies the immune response induced against the immunogen. Modification of the immune response may include intensification or broadening the specificity of either or both antibody and cellular immune responses. The rSARS-CoV-2 compositions described herein function as immunogens and can be administered as vaccines or immunogenic compositions. These vaccines or immunogenic compositions can contain an adjuvant and/or a pharmaceutically acceptable buffer.

CoVs are enveloped, single-stranded, positive-sense RNA viruses belonging to the Nidovirales order and responsible for causing seasonal mild respiratory illness in humans (e.g., 229E, NL63, OC43, HKU1). However, two previous CoVs have been associated with severe illnesses and resulted in significant morbidity and mortality in humans. These were severe acute respiratory syndrome CoV (SARS-CoV) in 2002 and Middle East respiratory syndrome CoV (MERS-CoV) in 2012. Like that of SARS-CoV, the SARS CoV-2 genome is approximately 30,000 bases in length. Nonetheless, a feature of SARS-CoV-2 that is unique among known betacoronaviruses is the presence of a furin cleavage site in the viral spike (S) glycoprotein, a characteristic known to increase pathogenicity and transmissibility in other viruses.

Recombinant BAC vectors containing the recombinant SARS-CoV-2 (rSARS-CoV-2) are important tools to understand the mechanisms of viral infection, transmission, and pathogenesis, as well as to identify viral and host factors and interactions that control viral cell entry, replication, assembly, and budding. In addition, the rSARS-CoV-2 compositions with reporter genes are used in cell-based screening assays or in vivo models of infection for the rapid and easy identification of prophylactic and therapeutic approaches for the treatment of viral infections, as well as to generate attenuated forms of viruses for their implementation as safe, immunogenic, and protective live attenuated vaccines (LAVs).

Assembly of full-length cDNAs of viruses with a large viral genome in E. coli is very challenging technically due to the toxicity, instability, or both of sequences within the viral genome. Described here are methods of development of a BAC-based reverse genetics system for the recovery of rSARS-CoV-2 from transfected cells. Compositions and methods described herein facilitate the identification and characterization of antiviral agents and the development of LAVs for the treatment of SARS-CoV-2 infection and associated COVID-19 disease.

Compositions and methods described herein facilitate analysis of various aspects of SARS-CoV-2 infection, such as understanding the interactions between the viral and host factors that control viral cell entry, replication, assembly, and budding. Compositions and methods described herein can be used for the rescue of rSARS-CoV-2 with predetermined mutations in their genomes to examine their contribution to viral multiplication and pathogenesis. Compositions and methods described herein can be used for the development of cell-based approaches to interrogate individual steps in the life cycle of SARS-CoV-2 to identify the mechanism of action of viral inhibitors. Compositions and methods described herein can be used for the generation of rSARS-CoV-2 clones expressing reporter genes for their use in cell-based screening assays or in vivo models for the rapid and easy identification of viral inhibitors and/or neutralizing antibodies. Compositions and methods described herein can be used for the generation of rSARS-CoV-2 clones containing mutations in their viral genomes that result in attenuation for their implementation as safe, immunogenic, stable, and protective LAVs for the treatment of COVID-19 disease. Compositions described herein can be used as part of a kit for detecting neutralizing antibodies to COVID-19 disease.

Compositions disclosed here include replication competent rSARS-CoV-2 constructs expressing one or more reporters, such as a fluorescent protein (Venus and mCherry) or luciferase (Nluc). While fluorescent proteins provide an efficient way to track viral infections using microscopy, luciferase proteins are more readily quantifiable and therefore more amenable to HTS studies. In certain embodiments, these reporter genes were selected based on their distinctive fluorescent properties (Venus and mCherry) and because of their small size, stability, high bioluminescence activity, and ATP-independency (Nluc).

Recombinant viruses expressing a red fluorescent protein represent an advantage over those expressing GFP or mNeonGreen as many genetically modified cell lines and/or animals express green fluorescent proteins. Another limitation of green fluorescent proteins during in vivo imaging is the absorption of the fluorophores' excitation and emission by hemoglobin and autofluorescence of tissues. Recombinant viruses expressing red fluorescent proteins present a better option to combine with genetically modified GFP-expressing cell lines and/or animals and, based on their reduced autofluorescence background, to more accurately capture the dynamics of viral infection and replication.

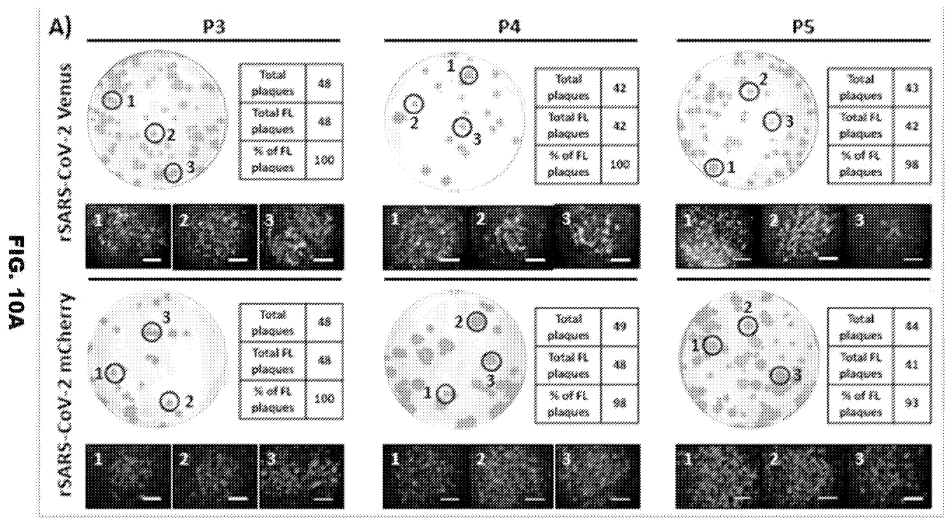

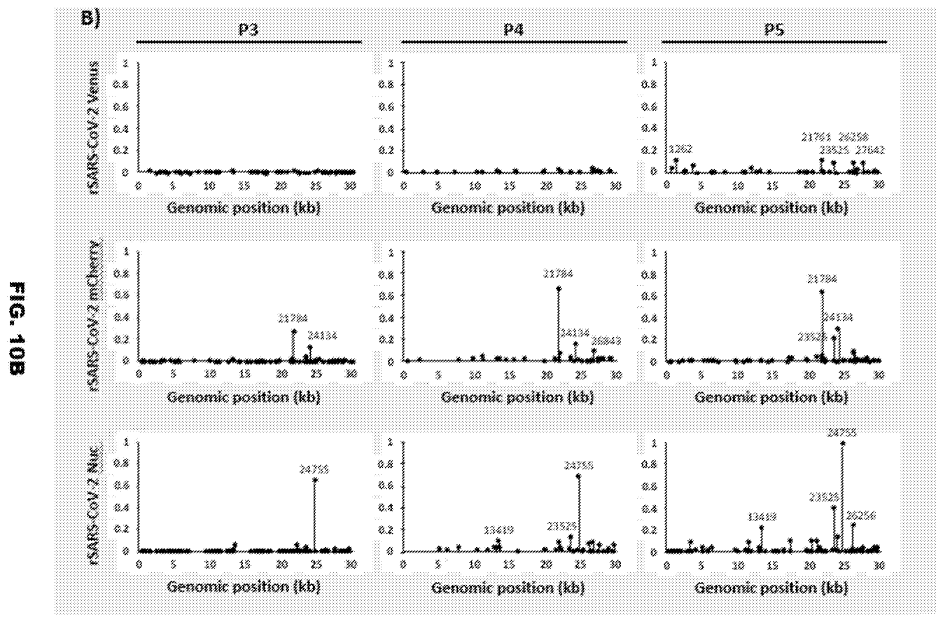

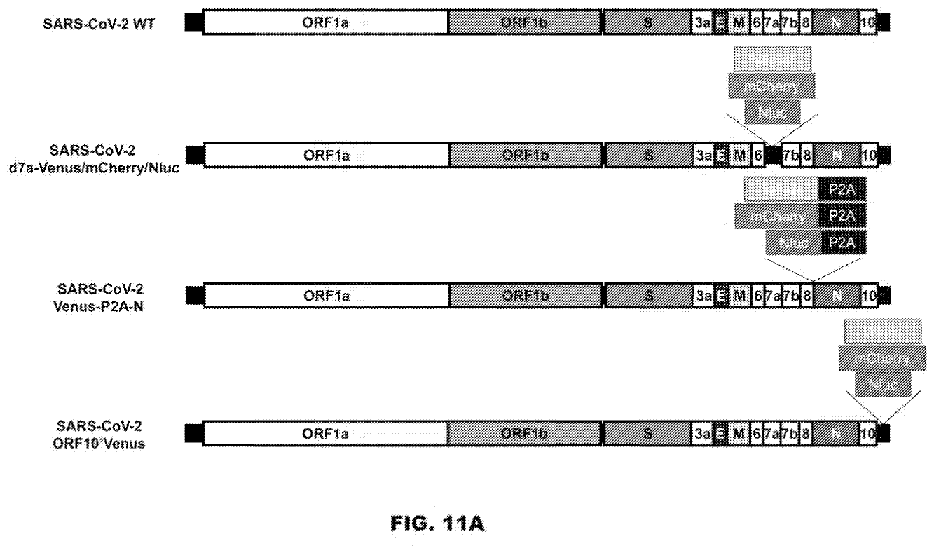

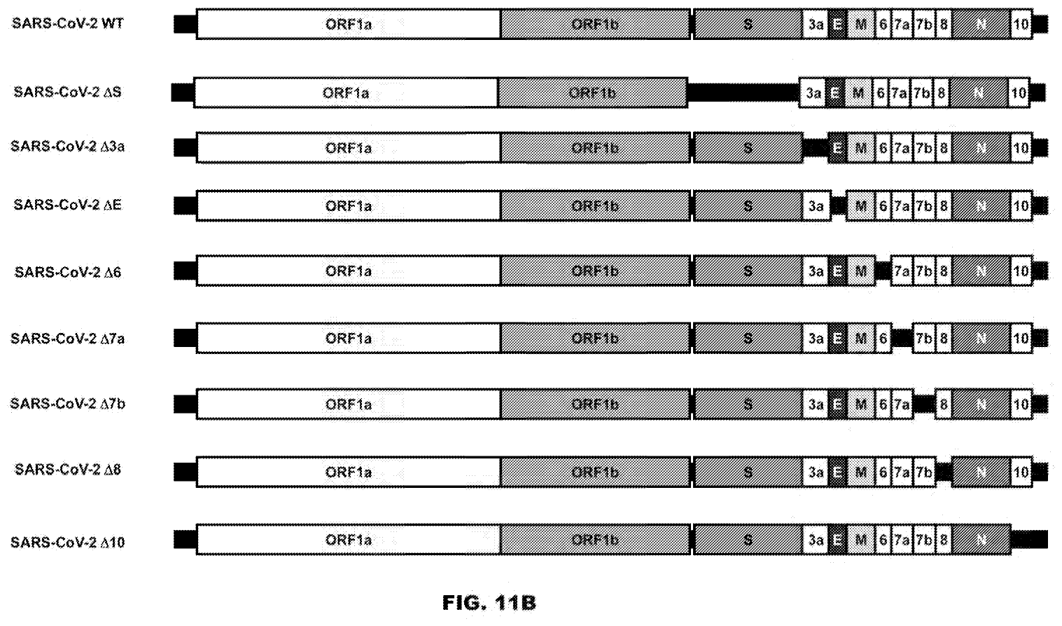

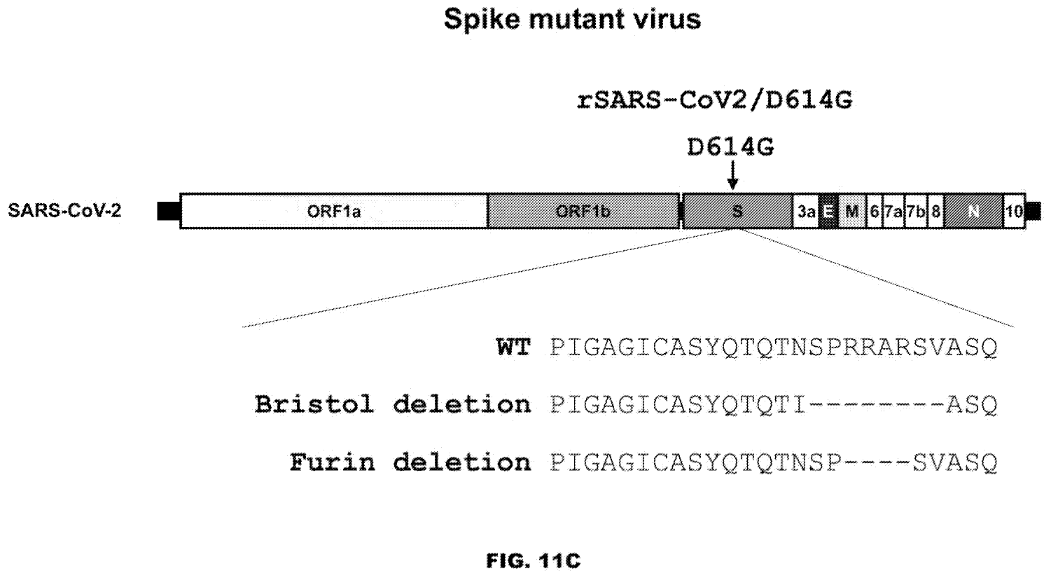

Reporter-expressing replicating competent viruses can be used to monitor viral infections, assess viral fitness, evaluate and/or identify antivirals and/or NAbs, where reporter gene expression can be used as a valid surrogate for viral detection in infected cells. Embodiments include rSARS-CoV-2 compositions developed to express one or more of Venus, mCherry, or Nluc reporters. FIG. 11A is a diagrammatic representation of the various Venus, mCherry, or Nluc expressing rSARS-CoV-2 compositions. Expression of Venus, mCherry, or Nluc from the rSARS-CoV-2 compositions were confirmed by directly visualizing fluorescence expression under a fluorescent microscope (Venus and mCherry) or luciferase activity (Nluc) using a microplate reader. Western blot analyses using specific antibodies against each of the reporter genes further confirm expression from their respective rSARS-CoV-2. Notably, despite deletion of the 7a ORF and insertion of a reporter gene, the three reporter-expressing rSARS-CoV-2 displayed similar growth kinetics and plaque phenotype as compared to their WT counterpart. These constructs facilitated the visualization of viral infection in real time, without the need of secondary approaches (e.g. MAbs) to detect the presence of the virus in infected cells. Overall, reporter gene expression displayed similar kinetics that correlated with levels of viral replication, further demonstrating the use of these reporter-expressing rSARS-CoV-2 constructs as a valid surrogate to assess viral infection.

There is a possibility, similar to the situation with other respiratory viruses (e.g. influenza), of the emergence of drug-resistant SARS-CoV-2 variants that will impose a significant challenge to the currently ongoing COVID-19 pandemic. Thus, it is imperative to not only develop new antivirals and other therapeutic approaches but also prophylactics for the treatment of SARS-CoV-2 infections. To that end, rapid and sensitive screening assays to identify chemical and biological agents with antiviral activity or to assess efficacy of vaccine candidates for the therapeutic and prophylactic treatment of SARS-CoV-2 infections, respectively, are urgently needed. The reporter-expressing rSARS-CoV-2 compositions facilitated the rapid identification and characterization of both antivirals and NAbs for the therapeutic and/or prophylactic treatment of SARS-CoV-2 infections. Importantly, the EC₅₀ (antivirals) and NT₅₀ (NAbs) obtained with the reporter-expressing rSARS-CoV-2 compositions were comparable to those obtained using rSARS-CoV-2/WT, demonstrating the use of the reporter-based microneutralization assays for the rapid identification of antivirals or NAbs. Furthermore, initial results indicate that reporter-expressing Venus, mCherry, and Nluc rSARS-CoV-2 were stable up to 5 passages in vitro in Vero E6 cells, including expression of the reporter gene. Similar to other respiratory viruses, that rSARS-CoV-2 expressing reporter genes can also be used to study the biology of viral infections in validated animals of viral infection.

Certain embodiments of the reporter-expressing rSARS-CoV-2 do not contain the 7a ORF and do not have any significant difference in viral replication—demonstrating the genetic plasticity of the SARS-CoV-2 genome. In an embodiment, similar to the removal of the viral genes and inserting reporter genes, the rSARS-CoV-2 compositions contain a gene of interest for the development of SARS-CoV-2 vaccines that could be used for the control of the currently ongoing COVID-19 pandemic. Recombinant SARS-CoV-2 can be generated by inserting the genes of interest (e.g. cytokines and/or chemokines) in the viral genome instead of the reporter genes. These cytokines and/or chemokines will help to stimulate innate and adaptive immune responses for the development of vaccines for the treatment of SARS-CoV-2 infection. Other foreign genes beside cytokines and/or chemokines can be similarly inserted in the SARS-CoV-2 genome, alone or in combination with deletions in the viral genes, for the development of LAVs.

Identification of SARS-CoV-2 neutralizing antibodies (NAbs) is important to assess vaccine protection efficacy, including their ability to protect against emerging SARS-CoV-2 Variants of Concern (VoC). To date, several VoC have been identified. VoC include the United Kingdom B.1.1.7 (alpha, α) 28, SA B.1.351 (β) 12, Brazil P.1 (gamma, γ) 29,30, California B.1.427 (epsilon, ε) 31, India B.1.617 (delta, δ), and B.1.1.529 (omicron, O). There is limited information on the ability of current vaccines to protect against these newly identified SARS-CoV-2 VoC. Moreover, it is plausible that new VoC continue to emerge in the future.

Recent evidence suggest that VoC are not efficiently neutralized by sera from naturally infected or vaccinated individuals, raising concerns about the protective efficacy of current vaccines against emerging SARS-CoV-2 VoC. Embodiments include methods of making and use of a rSARS-CoV-2 USA/WA1/2020 (WA-1) strain expressing Venus and a rSARS-CoV-2 expressing mCherry and containing mutations K417N, E484K, and N501Y found in the receptor binding domain (RBD) of the spike (S) glycoprotein of the South African (SA) B.1.351 (beta, β) VoC, in bifluorescent-based assays to rapidly and accurately identify human monoclonal antibodies (hMAbs) able to neutralize both in vitro and in vivo viral infections. Importantly, the bifluorescent-based assays accurately reflected the findings observed using individual viruses. Moreover, viruses with these novel rSARS-CoV-2 constructs had similar viral fitness in vitro to the parental wild-type (WT) rSARS-CoV-2 WA-1, as well as similar virulence and pathogenicity in vivo in the K18 human angiotensin converting enzyme 2 (hACE2) transgenic mouse model of SARS-CoV-2 infection. These fluorescent-expressing rSARS-CoV-2 constructs can be used in vitro and in vivo to easily identify hMAbs that simultaneously neutralize different SARS-CoV-2 strains, including VoC, for the rapid assessment of vaccine efficacy or the identification of prophylactic and/or therapeutic broadly MAbs.

To investigate in vitro and in vivo SARS-CoV-2 infection, including tissue and cell tropism and pathogenesis, recombinant (r)SARS-CoV-2 expressing fluorescent (Venus, mCherry, mNeonGreen, and GFP) or luciferase (Nluc) reporter genes have been developed and used for the identification of neutralizing antibodies (NAbs) or antivirals. Importantly, these reporter-expressing rSARS-CoV-2 viruses have been shown to have similar growth kinetics and plaque phenotype in cultured cells to those of their parental rSARS-CoV-2 wild-type (WT). Current rSARS-CoV-2 have been genetically engineered to express the reporter gene replacing the open reading frame (ORF) encoding for the 7a viral protein, an approach similar to that used with SARS-CoV.

Embodiments include constructs with the rSARS-CoV-2 expressing reporter genes where the porcine teschovirus 1 (PTV-1) 2A autoproteolytic cleavage site was placed between the reporter gene of choice and the viral nucleocapsid (N) protein. These embodiments were generated to take advantage of the following: (1) all viral proteins are expressed (e.g. the insertion of the reporter does not replace or remove a viral protein); (2) high levels of reporter gene expression from the N locus in the viral genome; and (3) high genetic stability of the viral genome in vitro and in vivo because of the need of the viral N protein for genome replication and gene transcription. These novel constructs allowed the visualization of infected cells in vitro and allow tracking SARS-CoV-2 infection in vivo. These reporter-expressing rSARS-CoV-2 viruses exhibited wild-type (WT)-like plaque size phenotype and viral growth kinetics in vitro, as well as pathogenicity in vivo.

Using the methods disclosed herein, Venus- and mCherry-expressing rSARS-CoV-2 USA/WA1/2020 (WA-1) and a rSARS-CoV-2 expressing mCherry and containing mutations K417N, E484K, and N501Y present in the receptor binding domain (RBD) of the viral spike (S) glycoprotein of the South Africa (SA) B.1.351 (beta, f) VoC were successfully rescued. Using rSARS-CoV-2 WA-1 expressing Venus and rSARS-CoV-2 SA expressing mCherry, a novel bifluorescent-based assay was developed to easily and accurately evaluate hMAbs able to specifically neutralize one or both viral variants. Importantly, the 50% neutralizing titers (NT₅₀) obtained with this bifluorescent-based assay correlated well with those obtained using individual viruses in separated wells. Moreover, rSARS-CoV-2 expressing different S and fluorescent proteins (FP) were used to rapidly identify hMAbs that are able to neutralize in vivo both SARS-CoV-2 strains using an in vivo imaging system (IVIS). These embodiments were used to investigate the efficacy of current and future SARS-CoV-2 vaccines, as well as the identification of hMAbs with broadly neutralizing activity against SARS-CoV-2 strains, including VoC, for the therapeutic or prophylactic treatment SARS-CoV-2 infection.

Reporter-expressing recombinant viruses circumvent limitations imposed by the need for secondary methods to detect the presence of viruses in infected cells. Novel rSARS-CoV-2 constructs have been generated to facilitate tracking of infection of two different SARS-CoV-2 strains (WA-1 and SA) in vitro and in vivo based on the use of two different FPs (Venus and mCherry). These FP-expressing rSARS-CoV-2 constructs encode the fluorescent Venus or mCherry proteins from the locus of the N protein, without the need of deletion of any viral protein. These constructs generated FP-expressing rSARS-CoV-2 that resulted in higher FP expression levels than those allowed by rSARS-CoV-2 expressing FPs from the locus of the viral ORF 7a protein. Moreover, rSARS-CoV-2 expressing reporter genes from the N locus are more genetically stable than those expressing reporter genes from the ORF7a locus of the SARS-CoV-2 genome.

The rSARS-CoV-2 expressing Venus or mCherry from the N locus exhibited similar growth kinetics, peak titers and plaque phenotype as the parental WT rSARS-CoV-2 WA-1 strain. These novel reporter rSARS-CoV-2 compositions were used in a bifluorescent-based assays to determine the neutralization efficacy of hMAbs based on FP expression levels. Compositions of rSARS-CoV-2 mCherry SA (a mCherry-expressing rSARS-CoV-2 containing the K417N, E484K, and N501Y mutations in the RBD of the S glycoprotein of the SA VoC) were generated. The rSARS-CoV-2 mCherry SA had a higher fitness that rSARS-CoV-2 Venus in cultured cells as determined by higher viral titers and a bigger plaque size phenotype. When used in the bifluorescent-based assay, hMAb 1212C2 was unable to neutralize rSARS-CoV-2 mCherry SA but it was able to efficiently neutralize rSARS-CoV-2 Venus. In contrast, hMAb 1213H7 displayed efficient neutralization of both rSARS-CoV-2 Venus and rSARS-CoV-2 mCherry SA. These in vitro results correlated with in vivo studies in which K18 hACE2 transgenic mice pre-treated with 1212C2 were protected against challenge with rSARS-CoV-2 Venus but not rSARS-CoV-2 mCherry SA, alone or in combination; while mice treated with 1213H7 were protected against lethal challenge with both reporter-expressing rSARS-CoV-2. These protection results were further corroborated using IVIS in which fluorescence and viral titers demonstrate the neutralizing protective efficacy of 1212C2 against rSARS-CoV-2 Venus but not rSARS-CoV-2 mCherry SA, while 1213H7 efficiently protected mice against challenge with both viruses, alone or in combination. These results demonstrate the use of both rSARS-CoV-2 Venus and rSARS-CoV-2 mCherry SA to accurately assess the ability of hMAbs to efficiently neutralize one or both SARS-CoV-2 strains, alone or in combination, in vitro and/or in vivo, and that the results obtained in these bifluorescent-based assays correlate well with those using individual viral infections.

Embodiments include methods of making and using two rSARS-CoV-2 expressing different FP and S glycoproteins in a bifluorescent-based assay to identify NAbs exhibiting differences in their neutralizing activity against different SARS-CoV-2 strains present in the same biological sample in vitro and in vivo. These methods were used to identify broad NAbs against different SARS-CoV-2 VoC by generating rSARS-CoV-2 expressing additional FP and containing the S glycoproteins of different VoC in multiplex-based fluorescent assays in vitro and/or in vivo. These reporter rSARS-CoV-2 expressing the S glycoprotein of VoC are used to investigate viral infection, dissemination, pathogenesis and therapeutic interventions, including protective efficacy of vaccines or antivirals, for the treatment of SARS-CoV-2 infection.

The reverse-genetics system approach was used to successfully engineer recombinant SARS-CoV-2 (rSARS-CoV-2) constructs. The SARS-CoV-2 genome encodes 16 nonstructural proteins (NSPs) and six accessory proteins, each encoded by independent open reading frames (ORFs). Both coronavirus NSP and ORF proteins play important roles in viral replication and transcription, evasion of host immune responses, and viral dissemination. In particular, SARS-CoV ORF3a has been implicated as an inducer of membrane rearrangement and cell death. SARS-CoV ORF3b, ORF6, and nucleocapsid (N) proteins have been described to counteract the host immune interferon (IFN) responses. SARS-CoV ORF7a protein has also been shown to inhibit cellular protein synthesis and activation of p38 mitogen-activated protein kinase. Conversely, coronavirus ORF7b has been reported to have a Golgi localization signal, where it becomes incorporated into the virion; however, no additional studies have delved into any further functions. Early clinical isolates identified a 382-nucleotide deletion leading to a truncated ORF7b protein and removal of the ORF8 transcription signal. This SARS-CoV-2 ORF8 has been postulated to play a minor role in disease outcome, as natural SARS-CoV-2 strains containing deletions in ORF8 have been isolated from individuals presenting with COVID-19.

Individual viral ORF3a, 26, 27a, 27b, and 28 proteins were deleted, and the resulting recombinant viruses were characterized in vitro and in vivo. Differences in plaque morphology, with ORF-deficient (ΔORF) viruses were observed, such as production of smaller plaques than those of the wild type (rSARS-CoV-2/WT). However, growth kinetics of ΔORF viruses were similar to rSARS-CoV-2/WT. Infection of K18 human angiotensin-converting enzyme 2 (hACE2) transgenic mice with the ΔORF rSARS-CoV-2s identified ORF3a and ORF6 as the major contributors of viral pathogenesis, while ΔORF7a, ΔORF7b, and ΔORF8 rSARS-CoV-2s induced pathology comparable to that of rSARS-CoV-2/WT. The reverse-genetics system was sufficiently robust to generate rSARS-CoV-2 constructs and dissect the major role for ORF3a and ORF6 in viral pathogenesis, providing important information for the generation of attenuated forms of SARS-CoV-2 for their implementation as live attenuated vaccines for the treatment of SARS-CoV-2 infection and associated COVID-19.

Embodiments described here are used to determine the contribution of SARS-CoV-2 accessory open reading frame (ORF) proteins to viral pathogenesis and disease outcome. Embodiments described here are used to develop a synergistic platform combining the robust reverse-genetics system to generate recombinant SARS-CoV-2 constructs. The SARS-CoV-2 ORF3a and ORF6 contribute to lung pathology and ultimately disease outcome in K18 hACE2 transgenic mice, while ORF7a, ORF7b, and ORF8 have little impact on disease outcome. Moreover, the combinatory platform described herein facilitate generation of attenuated forms of the virus to develop live attenuated vaccines for the treatment of SARS-CoV-2.

To examine the contribution of accessory proteins in the pathogenicity of SARS-CoV-2, the reverse-genetics approach based on the use of a BAC was used to rescue rSARS-CoV-2 with deletions of individual accessory ORF proteins. The rSARS-CoV-2 constructs deficient in ORF3a, ORF6, ORF7a, ORF7b, and ORF8 individually were successfully rescued. The in vitro characterization of each deficient-ORF (ΔORF) virus revealed a distinct difference in plaque phenotype from that of the recombinant wild-type SARS-CoV-2 (rSARS-CoV-2/WT). This phenotypic change, however, did not affect the viral growth kinetics. All ΔORF rSARS-CoV-2s replicated similarly to the rSARS-CoV-2/WT. Interestingly, changes in pathogenesis were observed between the WT and ΔORF rSARS-CoV-2s in the established K18 human angiotensin-converting enzyme 2 (hACE2) transgenic mouse model of SARS-CoV-2 infection. In contrast to the WT and the ORFΔ7a, ORFΔ7b, and ORFΔ8 rSARS-CoV-2s, the ORFΔ3a and ORFΔ6 rSARS-CoV-2s induced less pathology and resulted in 75% and 50% survival rates, respectively. Furthermore, both the ORFΔ3a and ORFΔ6 rSARS-CoV-2s had lower viral titers (102 PFU/ml) at 2 days post infection (p.i.). By 4 days p.i., the ORFΔ3a and ORFΔ6 rSARS-CoV-2s were no longer detected in nasal turbinates. In contrast, ORFΔ6 viral strain replication in the lungs reached 10⁵ PFU/ml at 2 days p.i. and only decreased by 2 log₁₀ at 4 days p.i. ORFΔ3a virus replication reached only 102 PFU/ml at 2 days p.i. and was not detected by 4 days p.i. in the lungs. Both the ORFΔ7a and ORFΔ7b rSARS-CoV-2s induced pathologies similar to that produced by rSARS-CoV-2/WT and resulted in a 25% survival rate. Based on the in vitro and in vivo data, live attenuated vaccines against SARS-CoV-2 were designed and developed.

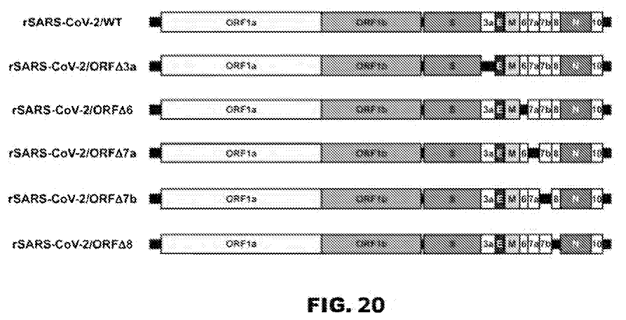

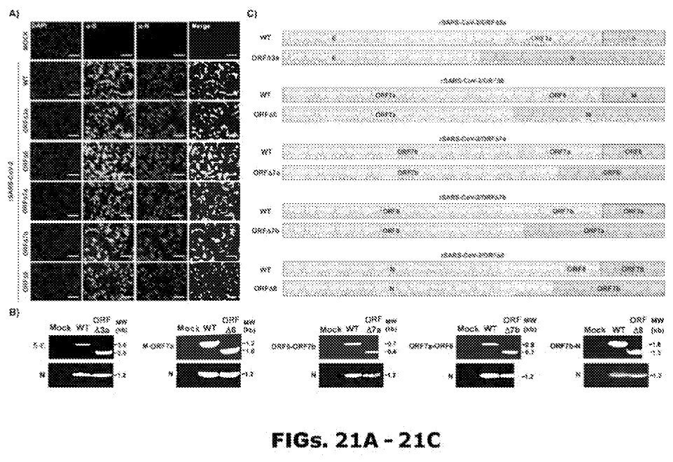

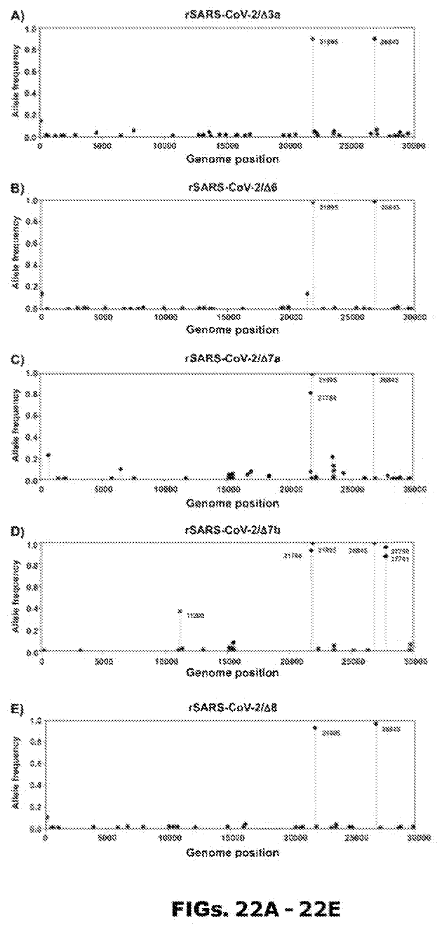

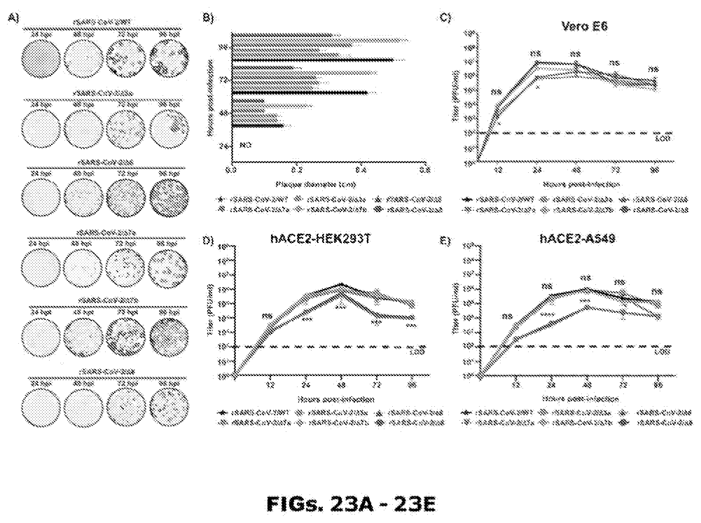

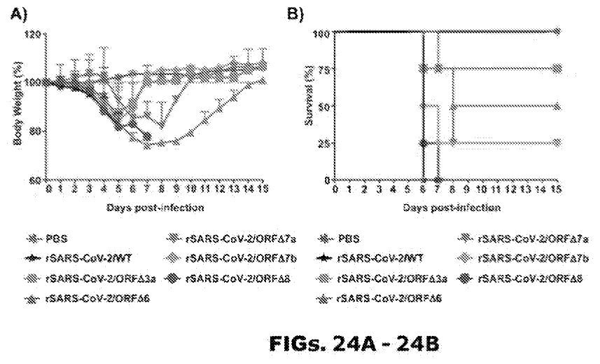

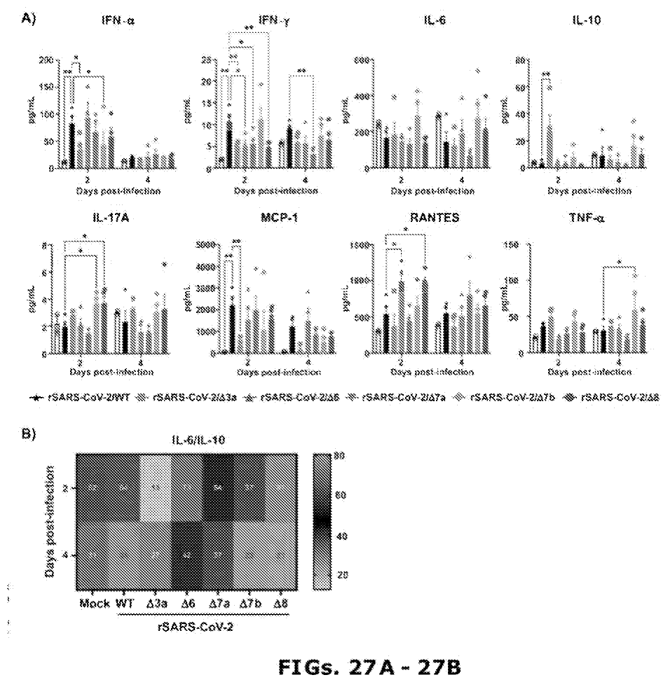

Here, rSARS-CoV-2 constructs deficient in the ORF3a, 26, 27a, 27b, or 28 accessory proteins were generated (FIG. 20) and characterized both in vitro (FIGS. 21A-21C and 23A-23E) and in vivo (FIGS. 24A-24B and 27A-27B). The ORF-deficient nature of these rSARS-CoV-2s were confirmed (FIGS. 21A-21C and 22A-22E). During the initial in vitro characterization, ORF proteins contributed to early dissemination and formation of detectable viral plaques in Vero E6 cell monolayers; viruses lacking the ORF3a, 27a, 27b, and 28 proteins developed smaller plaques than rSARS-CoV-2/WT (FIGS. 23A-23E). Surprisingly, only a 1-log₁₀ difference in growth kinetics was observed between the WT and any of the ΔORF rSARS-CoV-2s (FIGS. 23A-23E). Additionally, the variation in plaque morphology and size was indicative of ORF deletions having an impact on viral dissemination and fitness. This correlates with other studies that have correlated plaque size phenotype and size with virulence and viral fitness.

To further determine the contribution of each ORF accessory protein in SARS-CoV-2 pathogenesis, the K18 hACE2 transgenic mouse model was used (FIGS. 24A-24B). A broad range of morbidity and survival outcomes was observed between each of the ΔORF rSARS-CoV-2s and rSARS-CoV-2/WT (FIGS. 24A-24B). SARS-CoV-2 ORF accessory proteins are encoded in the following order: ORF3a, 26, 27a, 27b, and 28. Data here demonstrated the greatest survival in mice infected with ORFΔ3a rSARS-CoV-2 (75%) and 100% mortality with ORFΔ8 rSARS-CoV-2. A natural SARS-CoV-2 variant with a deletion of ORF8 has recently been isolated from patients presenting with COVID-19 symptoms. Thus, data from ORFΔ8 rSARS-CoV-2 in the K18 hACE2 transgenic mouse model correspond to those observed in people infected with a natural ORFΔ8 SARS-CoV-2 isolate. As it is well known that SARS-CoV-2 ORF6 is a potent inhibitor of the host innate immune response, low viral loads and an increased immune response with ΔORF6 rSARS-CoV-2 were expected; however, as SARS-CoV N protein also inhibits host immune responses, it appears that SARS-CoV-2 N protein may have a function similar to that of OFR6 and may be responsible for counteracting host innate immune and inflammatory responses. An increase in immune responses may in turn correlate with the decrease in weight loss and recovery in the mice infected with ΔORF6 rSARS-CoV-2. The chemokine and cytokine analysis indicates that, specifically, ORF3a is implicated in driving the host immune response to SARS-CoV-2 during the early stages of infection. Indeed, in the absence of ORF3a, a general decrease of the cytokine storm (IL-6/IL-10 ratio) was observed, especially at 2 days p.i., when significantly decreased viral titers and tissue damage were also observed in the lungs of animals infected with ΔORF3a rSARS-CoV-2. SARS-CoV ORF3a, 23b, 26, 27a, and 27b had no significant impact on viral replication in vivo, but ORF3a seems to be involved in virulence, as its absence decreases SARS-CoV-2 virulence.

The examples below demonstrate the robustness of the BAC-based reverse-genetics approach to generate rSARS-CoV-2s, including those with deletions of ORF accessory proteins, and provide information on the contributions of ORF3a, 26, 27a, 27b, and 28 accessory proteins in viral fitness in vitro (Vero E6 cells) and in vivo in the K18 hACE2 transgenic mouse model of SARS-CoV-2 infection and COVID-19 disease. Embodiments include attenuated forms of SARS-CoV-2 for the development of live attenuated vaccines for the treatment of this important respiratory pathogen and its associated COVID-19 disease.

Recombinant SARS-CoV-2 with double deletions of accessory ORF proteins were rescued. The SARS-CoV-2 genome, which was divided into 5 fragments and chemically synthesized, was assembled into a single BAC that led to efficient virus rescue after being transfected into Vero E6 cells. The recombinant (r)SARS-CoV-2 constructs with individual deletion of accessory ORF proteins retain virulence in K18 hACE2 transgenic mice; however, they were attenuated partly, particularly the rSARS-CoV-2/Δ3a. The backbone of Fragment 1 with deletion of ORF3a was used to introduce an additional deletion of either ORF6, ORF7a, ORF7b or ORF8. By using the same approach described previously, double deletion of ORF3a/ORF6, ORF3a/ORF7a, ORF3a/ORF7b and ORF3a/ORF8 were successfully introduced into the Fragment 1. After being confirmed by sanger sequencing, the fragments containing these combinational deletions were reassembled into the BAC. Each BAC with double deletions in the accessory ORFs were transfected into Vero E6 cells for the recovery of double ΔORF rSARS-CoV-2. At 72 h post-transfection, tissue culture supernatants (P0) were collected to infect fresh Vero E6 cells to generate P1 stocks. All the double deletion rSARS-CoV-2 except ΔORF3a/ΔORF8 were successfully rescued.

Mice infected with double deletions rSARS-CoV-2 Δ3a/Δ6 and rSARS-CoV-2 Δ3a/Δ7b showed no signs of disease and had a 100% survival rate. In contrast, rSARS-CoV-2 Δ3a/Δ7a infection resulted in a 25% lethality occurring at 10 days post infection. Total IgG ELISAs and microtiter neutralization assays identified that both rSARS-CoV-2 Δ3a/Δ7a and rSARS-CoV-2 Δ3a/Δ7b induced antibody responses against SARS-CoV-2 WA-1/2020 and emerging variants, while the rSARS-CoV-2 Δ3a/Δ6 did not. Further cytokine and chemokine analysis of lung homogenates identified that compared to the rSARS-CoV-2, the double ΔORF rSARS-CoV-2 mutants induced lower levels of chemo-attractants and IFN-α by day 4 post-infection. Moreover, rSARS-CoV-2 Δ3a/Δ6 was not detected at either 2 or 4 d p.i. in the nasal turbinates, while rSARS-CoV-2 Δ3a/Δ7a was detected at both 2 and 4 d p.i, and lastly rSARS-CoV-2 Δ3a/Δ7b was only detected at 2 d p.i. In the lungs, rSARS-CoV-2 Δ3a/Δ6 and rSARS-CoV-2 Δ3a/Δ7a were only detected at 2 d p.i., while rSARS-CoV-2 Δ3a/Δ7b maintained similar levels of replication to the rSARS-CoV-2 WT, ˜10⁵ PFU/mL at 2 d p.i. and decreasing to ˜10³ PFU/mL at 4 d p.i. Further studies with rSARS-CoV-2 Δ3a/Δ6 and rSARS-CoV-2 Δ3a/Δ7b in vivo identified that both of these induced protection against lethal challenge. Real-time infection monitoring of mice vaccinated with rSARS-CoV-2 Δ3a/Δ6 or rSARS-CoV-2 Δ3a/Δ7b and challenged with a lethal dose of rSARS-CoV-2 Nluc-2A (1×10⁵ PFU/mL) demonstrated that compared to the PBS vaccinated group, no challenge virus was detected at any day post infection (1, 2, 4, and 6) and mice from the vaccinated group had a 100% survival rate, while the PBS vaccinated group succumbed to infection by 8 days post challenge. These results were further confirmed as no challenge virus was detected in the nasal turbinates or lungs of the rSARS-CoV-2 Δ3a/Δ6 and rSARS-CoV-2 Δ3a/Δ7b vaccinated mice at 2 or 4 d p.i. Cytokine and chemokine analysis of the lung tissue homogenates collected at 2 and 4 d p.i. identified a delayed and controlled induction of cytokines and chemokines. Altogether, rSARS-CoV-2 Δ3a/Δ6 and rSARS-CoV-2 Δ3a/Δ7b can serve as LAVs against SARS-CoV-2.

Development of vaccines that confer long lasting immunity while proving to be 100% safe amongst the entire populace, are two of the most desired attributes for a vaccine candidate to have. Traditionally, inactivated or highly attenuated vaccine platforms have been used to develop vaccines against both positive and negative sense RNA viruses. Inactivation platforms require the use of fixatives and or chemical agents that render the pathogen inactive, however, in some instances this platform has failed to fully inactivate the agent and resulted in full infection of individuals. Moreover, during attenuation, LAVs are created through the serial passaging of pathogens in vitro or in vivo until genome mutations are widespread or the tropism of the pathogen changes. Despite their robust and longer lasting immune responses and protection, LAVs can have the possibility to revert to wild-type and cause adverse effects due to their change in tropism. New vaccine platforms such as mRNA, adenoviral, or viral like particle (VLP) have emerged, widening the potential for the development of safe and efficacious vaccines. Despite the use of these new platforms, the use of the viral glycoprotein remains a constant feature. This, in turn limits the capacity of these platforms to develop vaccines that elicit immune responses and antibodies against a single viral protein, and in most cases, is not enough to confer 100% protection from infection.

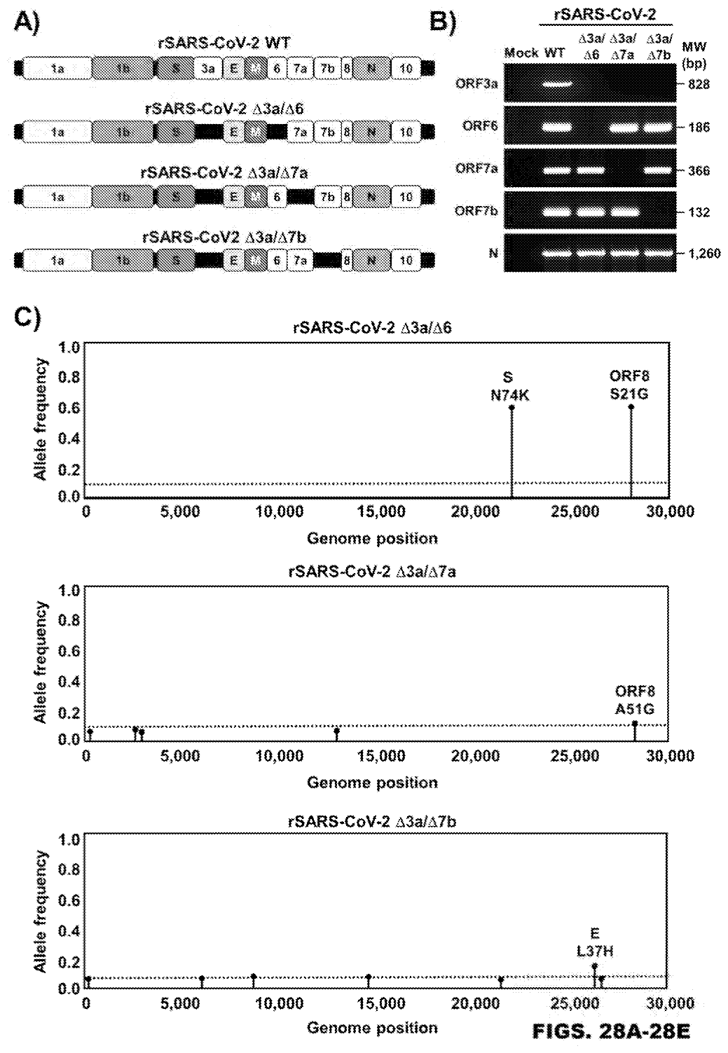

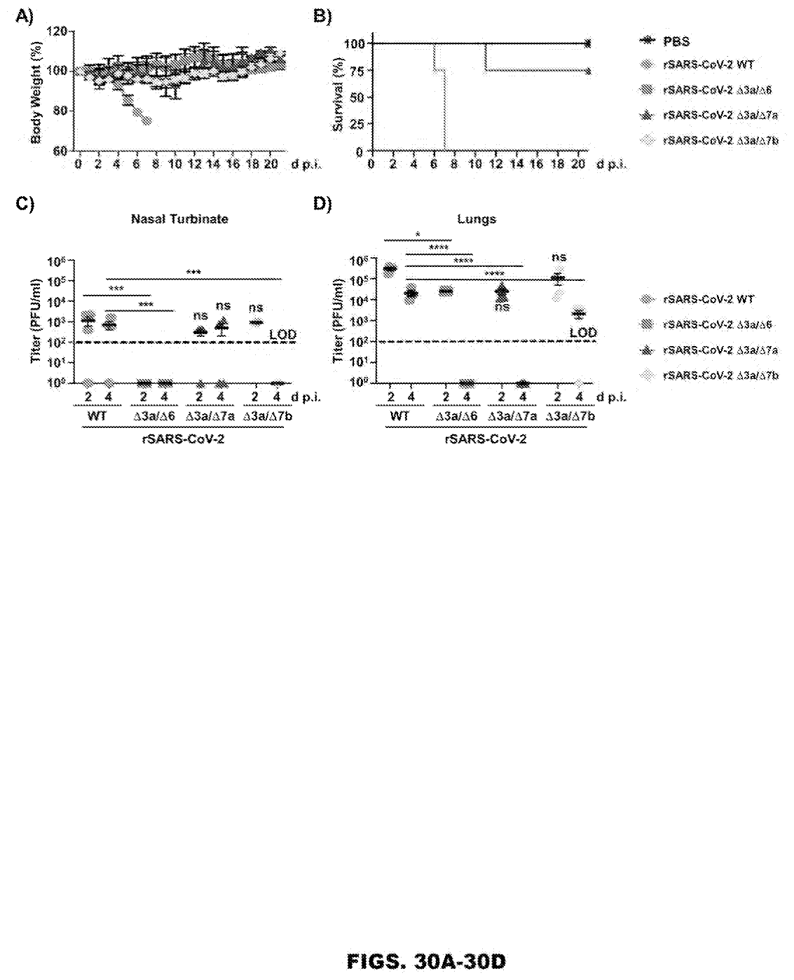

Embodiments include reverse genetics systems for generation of SARS-CoV-2 deficient in ORF3a, ORF6, ORF7a, ORF7b, and ORF8 and evaluation of these single ΔORF mutants for attenuation both in vitro and in vivo. Despite being modestly attenuated, all mutants proved lethal in K18 hACE-2 mice, the model of SARS-CoV-2 disease. Embodiments include fully attenuated LAV candidate SARS-CoV-2 constructs with double ΔORF mutant combinations of Δ3a/Δ6, Δ3a/Δ7a, or Δ3a/Δ7b (FIG. 28A). In vitro characterization of these double ΔORF mutants revealed attenuation in both VeroE6 (FIG. 28C) and hACE2-Δ549 (FIG. 28D) cell lines and an affected plaque morphology (FIG. 28A), correlating with other studies signifying viral attenuation. To identify an attenuated SARS-CoV-2, the K18 hACE2 transgenic mice were used for further studies (FIGS. 30A-30D). No significant changes in morbidity in mice infected with any of the double ΔORF rSARS-CoV-2 mutants were observed (FIG. 30A) and 100% survival in mice infected with the rSARS-CoV-2 ΔORF3a/Δ6 and rSARS-CoV-2 ΔORF3a/Δ7b, while the rSARS-CoV-2 ΔORF3a/Δ7a mutant resulted in 75% survival (FIG. 30B). Evaluation of viral titers in the nasal turbinate (FIG. 30C) and the lungs (FIG. 30D) at 2 and 4 d p.i. indicated that the rSARS-CoV-2 ΔORF3a/Δ6 failed to replicate, as it was not detected in the nasal turbinates at either day and only at 2 d p.i. in the lungs with a viral titer of 2×10⁴ PFU/mL, almost 1-log₁₀ lower than the original inoculum of 1×10⁵ PFU/mL. In contrast, the rSARS-CoV-2 ΔORF3a/Δ7a replicated in the nasal turbinates reaching viral titers up to 1×10³ PFU/mL by 4 d p.i., however, it was only detected at 2 d p.i. in the lungs. The rSARS-CoV-2 ΔORF3a/Δ7b mutant surprisingly replicated to titers like the wild-type rSARS-CoV-2 in the lungs at both 2 and 4 d p.i. but was only detected at 2 d p.i. in the nasal turbinates.

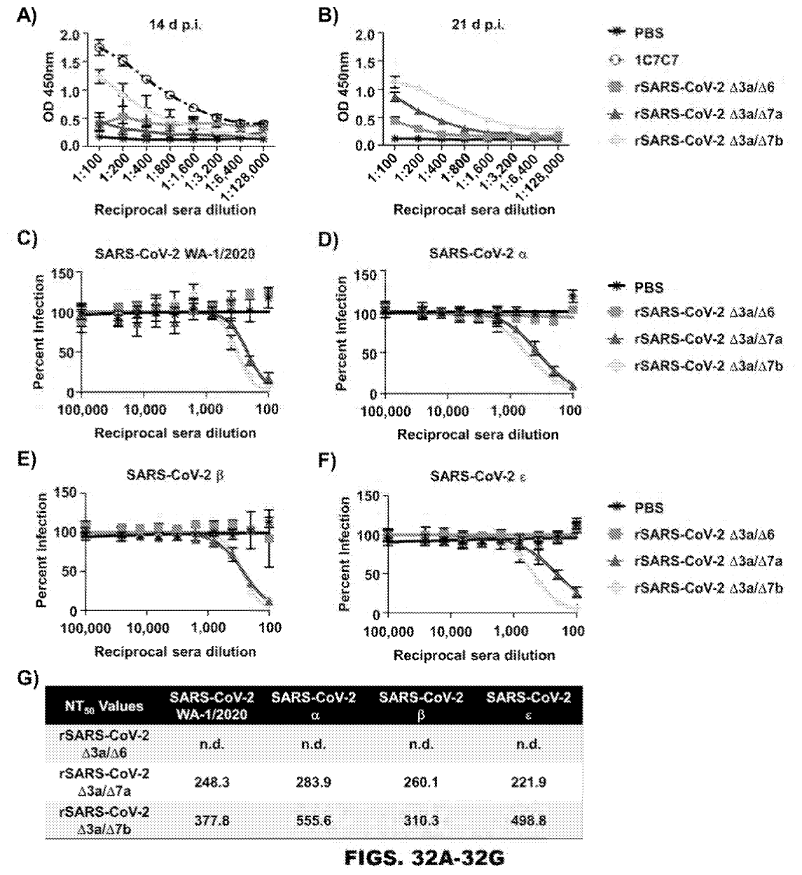

Embodiments also include the double ΔORF SARS-CoV-2 mutants, which induce antibodies capable of recognizing or neutralizing wild-type SARS-CoV-2 and emerging variants. For example, both the rSARS-CoV-2 ΔORF3a/Δ7a and rSARS-CoV-2 ΔORF3a/Δ7b mutants elicited antibodies against the SARS-CoV-2 WA-1/2020 natural isolate at 14 d p.i. (FIG. 32A) and these increased by 21 d p.i. (FIG. 32B), while rSARS-CoV-2 ΔORF3a/Δ6 failed to induce anti-SARS-CoV-2 antibody production. Plaque reduction micro-neutralization assays correlated with the ELISA data. Sera collected from both rSARS-CoV-2 ΔORF3a/Δ7a and rSARS-CoV-2 ΔORF3a/Δ7b infected mice at 21 d p.i., neutralized not only SARS-CoV-2 WA-1/2020 natural isolate but also emerging variants α, β, and ε (FIGS. 32C-32F, respectively). Cytokine and chemokine analyses of lung homogenates collected at 2 and 4 d p.i., indicate that rSARS-CoV-2 Δ3a/Δ7a mutant is capable of blocking IFN type I and II production, while inducing production of Th1/Th2/Th17 responses with an IL-6/IL-10 ratio lower than the rSARS-CoV-2 WT. This embodiment induces a local immune response that is beneficial for reducing or delaying the generation of the cytokine storm in the host (e.g., lower IFNs, lower IL-6, and lower chemo-attractant production).

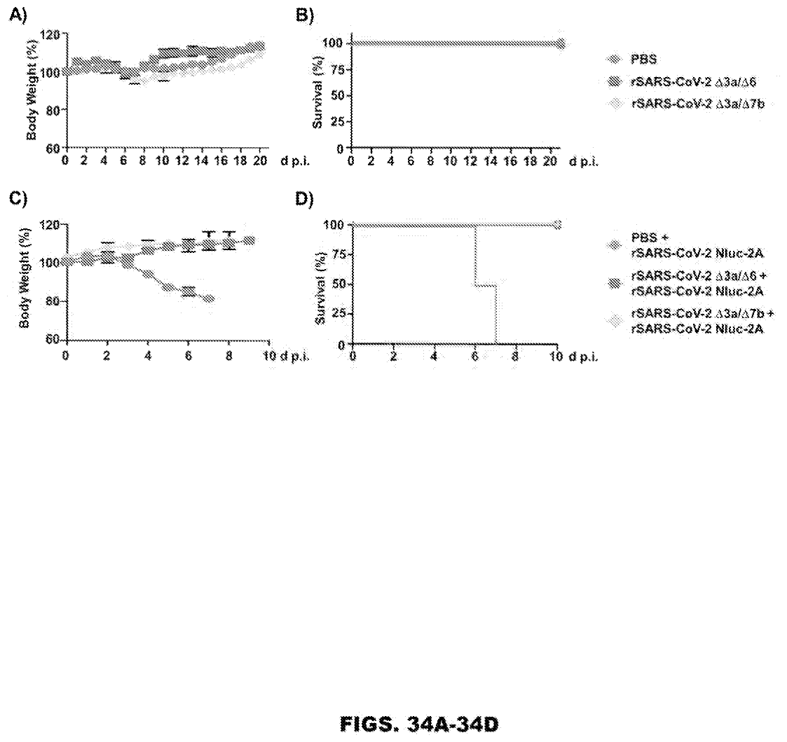

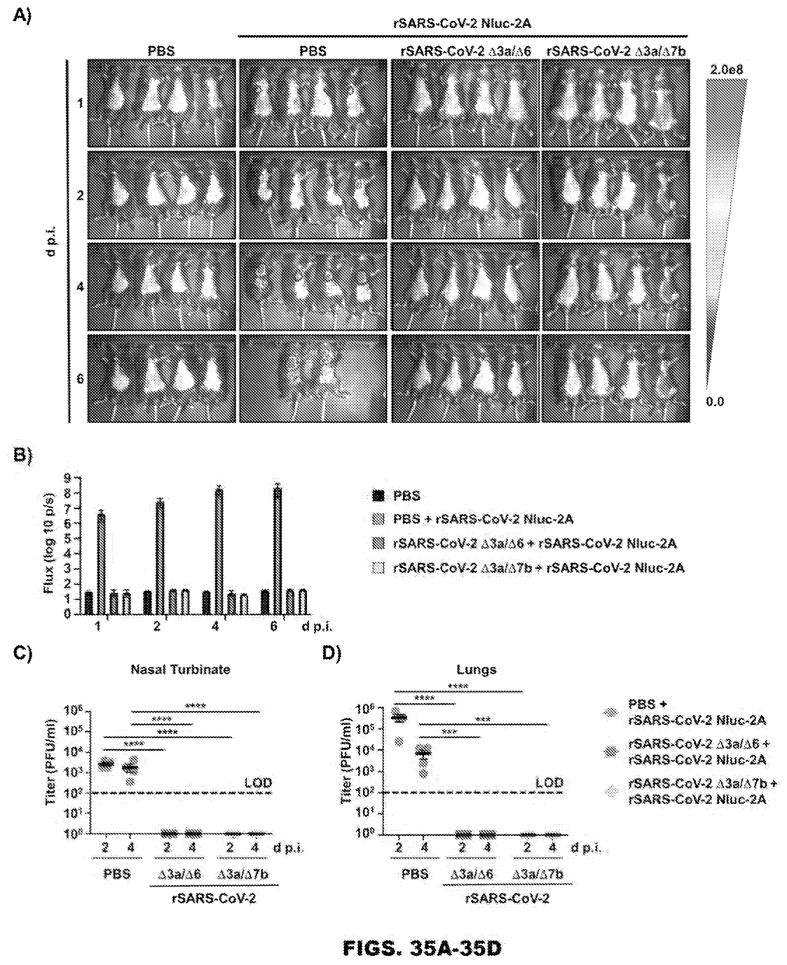

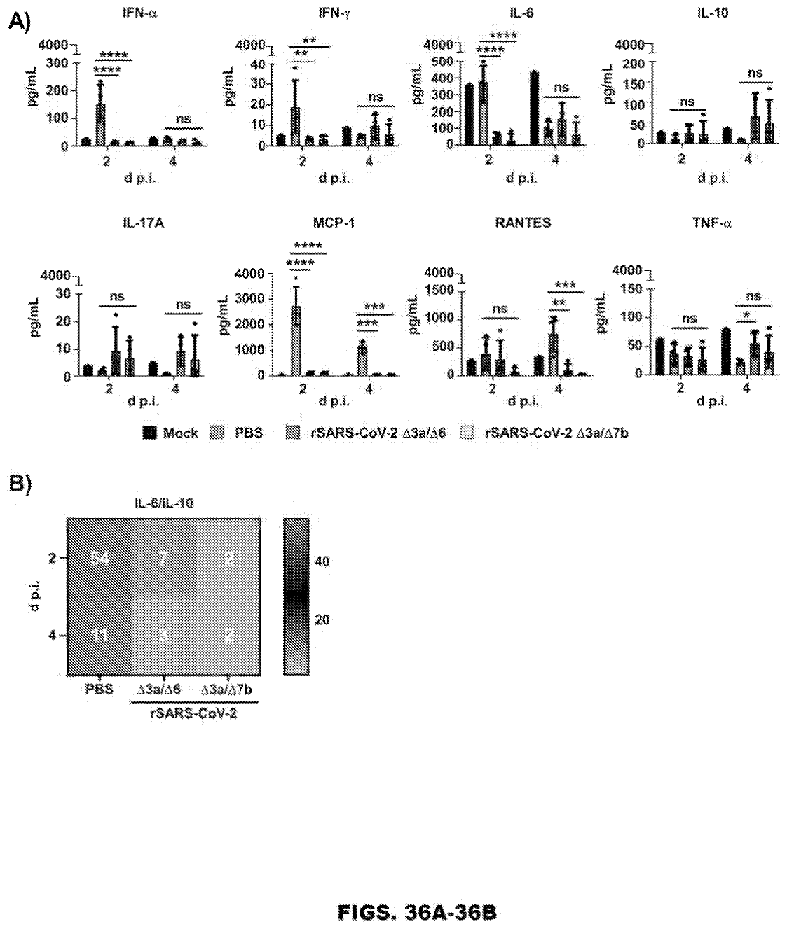

Embodiments also include the use of rSARS-CoV-2 ΔORF3a/Δ6 and rSARS-CoV-2 ΔORF3a/Δ7b constructs as LAVs against SARS-CoV-2. All of the mice vaccinated with rSARS-CoV-2 ΔORF3a/Δ6 survived and showed no signs of morbidity (FIGS. 34C and 34D, respectively). The mice vaccinated with rSARS-CoV-2 ΔORF3a/Δ7b also survived the challenge. Using rSARS-CoV-2 Nluc-2A allowed for the monitoring of infection in real-time in the vaccinated mice (FIGS. 35A-35D). In contrast to the groups vaccinated with the double ΔORF rSARS-CoV-2 mutants, rSARS-CoV-2 Nluc-2A replication was visible as soon as 1 d p.i. in the PBS vaccinated group and infection increased up to 6 d p.i., when all the mice succumbed to infection. Viral titers from nasal turbinates and lung collected from the PBS, rSARS-CoV-2 ΔORF3a/Δ6, and rSARS-CoV-2 ΔORF3a/Δ7b vaccinated groups at 2 and 4 d p.i. correlated with the real-time infection monitoring, with rSARS-CoV-2 Nluc-2A only being detected in the PBS vaccinated group (FIG. 35C). Cytokine and chemokine analysis of lung homogenate collected at 2 and 4 days post challenge (FIG. 36A) revealed that rSARS-CoV-2 ΔORF3a/Δ6 and rSARS-CoV-2 ΔORF3a/Δ7b vaccinated groups, upon challenge, produced lower levels of IFN responses accompanied with significant decreases in chemokine production and a lower IL-6/IL-10 ratio (FIG. 36B). These results are consistent with an attenuated cytokine storm, which may explain the reduction in tissue damage observed and the better control of the infection by mice. The later TH1 response observed at 4 d p.i. (e.g. TNF and IFN-γ) indicates that vaccination with rSARS-CoV-2 ΔORF3a/Δ6 and rSARS-CoV-2 ΔORF3a/Δ7b may result in the attenuation of the rapid evolution of the detrimental cytokine response observed for PBS vaccinated group.

Embodiments include LAVs against SARS-CoV-2 that are generated by deletion of ORFs that contribute to pathogenesis. By deletion of entire ORF regions, these two LAVs lack the potential for reversion to wild-type. Unlike other groups that have developed cold-adapted SARS-CoV-2 with genome wide mutations or recombinant viruses expressing the spike protein of SARS-CoV-2, the double ΔORF rSARS-CoV-2 mutants have no significant genome mutations and express all SARS-CoV-2 proteins that induce antigenicity, N, S, M, and E.

The following examples are provided to illustrate, but not to limit, the various embodiments of the compositions and methods described in this disclosure.

### EXAMPLES

### Example 1

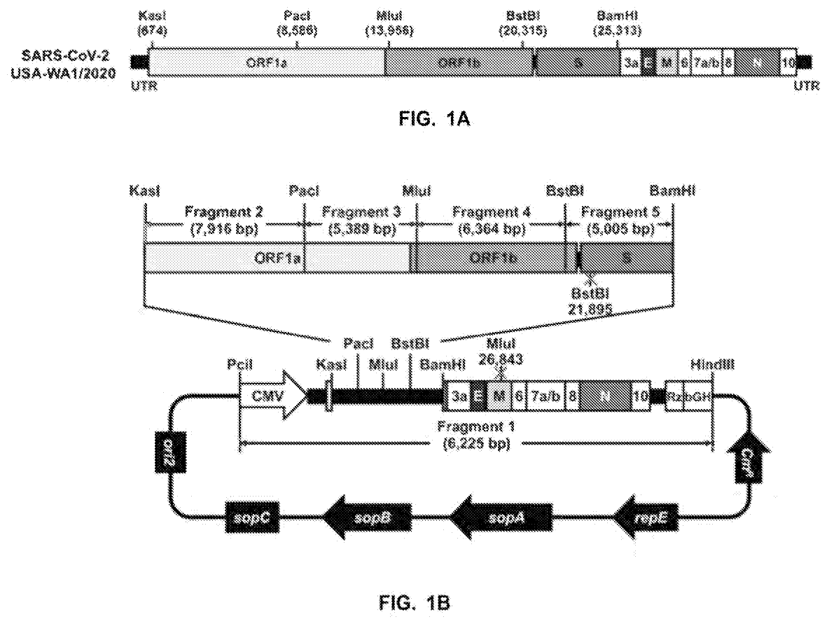

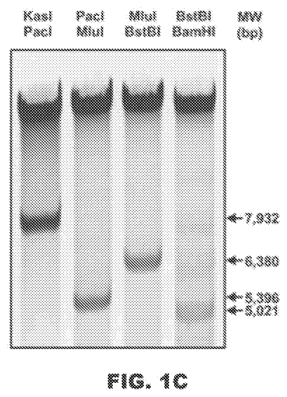

Assembly of the SARS-CoV-2 genome in the BAC. A BAC-based approach was used to assemble an infectious clone of SARS-CoV-2 based on the USA-WA1/2020 strain (FIG. 1A). This SARS-CoV-2 strain was isolated from an oropharyngeal swab from a patient with respiratory illness in Snohomish County, Washington state, USA. The viral sequence was deposited in PubMed [GenBank: MT576563.1] and the virus isolate is available from BEI Resources [NR-52281].

FIG. 1A is a schematic representation of the SARS-CoV-2 genome. The indicated restriction sites were used for cloning the entire viral genome (29,903 nucleotides) of SARS-CoV-2, USA-WA1/2020 strain, into the pBeloBAC11 plasmid. The open reading frames of the viral structural 1a, 1b, spike (S), envelop (E), matrix (M), and nucleocapsid (N) proteins and the accessory (3a, 6, 7a, 7b, 8, and 10) proteins are also indicated. UTR, untranslated regions. Length is not to scale. The entire viral genome was chemically synthesized in 5 fragments, which were assembled in the pBeloBAC plasmid using unique restriction enzymes and standard molecular biology approaches (FIG. 1B). FIG. 1B is a schematic representation of the full-length infectious cDNA clone as assembled by sequentially cloning chemically synthesized fragments 1 to 5, which cover the entire viral genome, into the pBeloBAC11 plasmid by using the indicated restriction sites under the control of the cytomegalovirus (CMV) promoter; the clone was flanked at the 3′ end by the hepatitis delta virus (HDV) ribozyme (Rz) and the bovine growth hormone (bGH) termination and polyadenylation sequences. The length of each of the chemically synthesized viral fragments is indicated. Ori2 indicates the origin of the replication of BAC. sopA, sopB, and sopC are the elements to ensure that each bacterial cell gets a copy of the BAC. Cm^{R} indicates chloramphenicol resistance. After assembly of the 5 fragments, the BAC containing the entire viral genome was analyzed by restriction enzyme analysis (FIG. 1C). FIG. 1C is a photographic representation of the analysis of the BAC clone harboring the entire viral genome after digestion with the indicated restriction enzymes (top), as analyzed in a 0.5% agarose gel. To facilitate the assembly of the viral genome and incorporate genetic tags to distinguish the rSARS-CoV-2 clone from the natural isolate, two silent mutations were introduced in the viral genes for S (21,895 nucleotides [nt]) and matrix (M) (26,843 nt) that removed BstBI and MluI restriction sites, respectively (FIG. 1B).

### Example 2

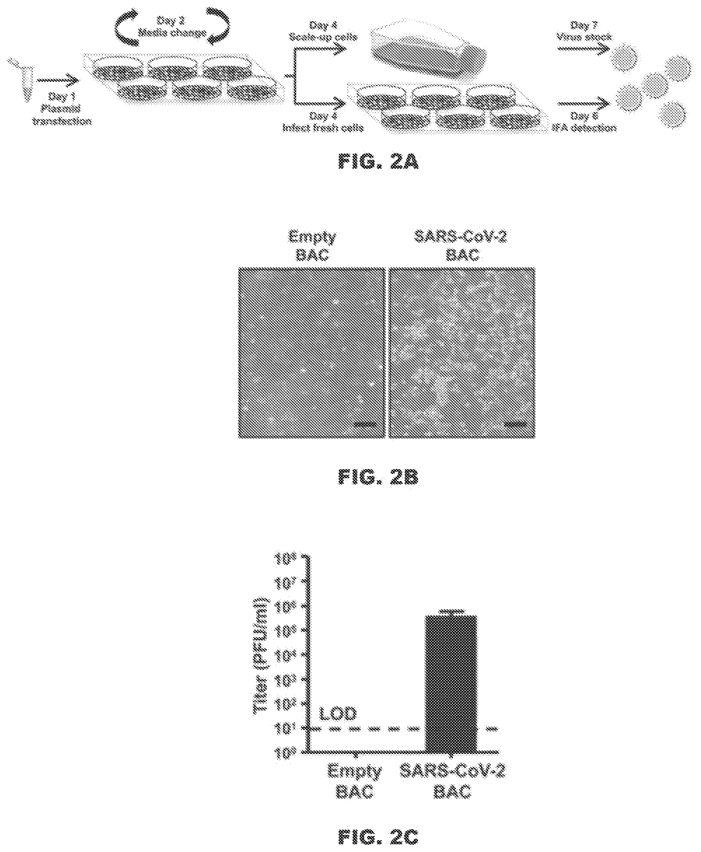

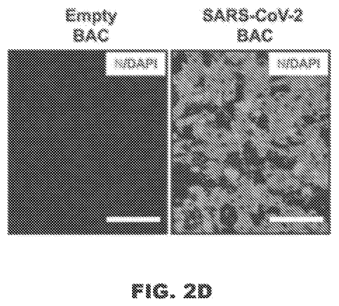

Rescue of rSARS-CoV-2. To recover rSARS-CoV-2, an experimental approach was used as described in FIG. 2A. FIG. 2A is a schematic representation of the method used to generate rSARS-CoV-2 compositions. Vero E6 cells were transiently transfected with the SARS-CoV-2 BAC at day 1. After 24 h, transfection medium was changed to post infection medium. At day 4, cells were split into T75 flasks and the tissue culture supernatant was used to infect fresh Vero E6 cells. At 48 h post infection, Vero E6 cells were fixed for detection of rSARS-CoV-2 by immunofluorescence, and the tissue culture supernatant of the scaled-up Vero E6 cells was collected at 72 h. As an internal control for this experiment, Vero E6 cells were transfected with the empty BAC. Vero E6 cells were transfected with the SARS-CoV-2 BAC or an empty BAC as an internal control and were monitored for the presence of cytopathic effect (CPE), which was evident at 72 h posttransfection (FIG. 2B). FIG. 2B is a set of photographic images of empty BAC-transfected (left panel) or SARS-CoV-2 BAC-transfected (right panel) Vero E6 cells at 72 h post transfection to evaluate the cytopathic effect (CPE). Scale bars, 100 m. Production of infectious virus (designated passage 0 [P0]) by transfected cells was at 3.4×10⁵ PFU/ml (FIG. 2C). FIG. 2C is a graphical representation of the viral titers. Tissue culture supernatant from mock-infected (empty BAC) or transfected Vero E6 cells in T75 flasks was collected and titrated by immunofluorescence. Data are presented as means SDs. LOD, limit of detection. Recovery of rSARS-CoV-2 was confirmed by detection of viral antigen in fresh Vero E6 cells infected with tissue culture supernatants collected from SARS-CoV-2 BAC-transfected Vero E6 cells, but not from empty BAC-transfected Vero E6 cells, by immunofluorescence using a monoclonal antibody against the nucleocapsid (N) protein of SARS-CoV that cross-reacts with SARS-CoV-2 N (FIG. 2D). FIG. 2D is a set of photographic images following evaluation of empty BAC (left panel) or SARS-CoV-2 BAC (right panel) infected Vero cells as analyzed by an immunofluorescence assay. N protein (green), 4,6-diamidino-2-phenylindole (DAPI; blue). Scale bars, 100 m. Vero E6 cells infected with the tissue culture supernatants from transfected Vero E6 cells were fixed at 48 h post infection, and viral detection was carried out by using a SARS-CoV cross-reactive monoclonal antibody (1C7) against the N protein (green). Cellular nuclei were stained by 4=,6-diamidino-2-phenylindole (DAPI; blue). Scale bars, 100 m.

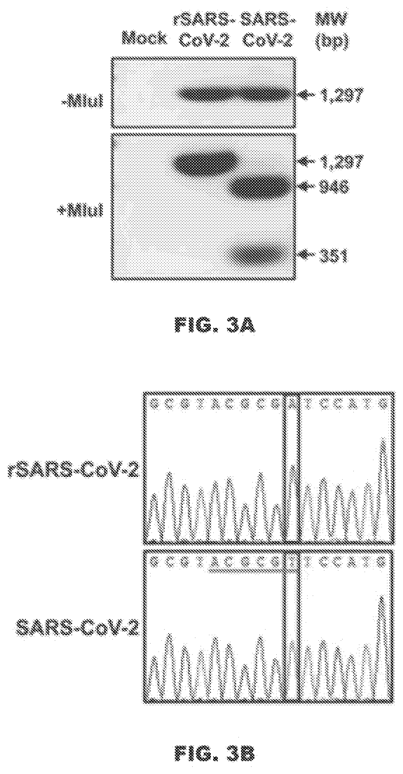

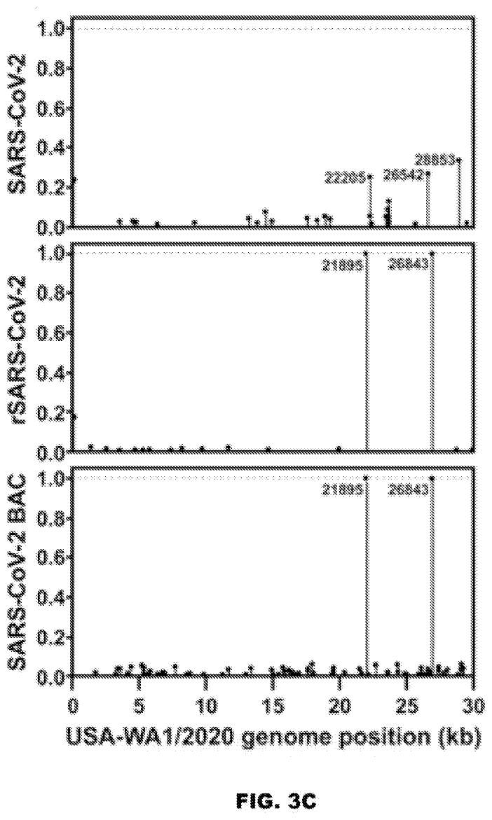

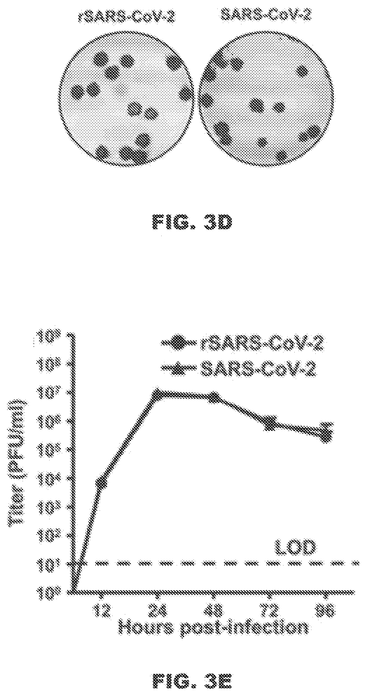

Characterization of rSARS-Co V-2 in vitro. The genetic identity of the rescued rSARS-CoV-2 clone was first confirmed. To that end, total RNA isolated from rSARS-CoV-2- and SARS-CoV-2-infected Vero E6 cells were used to amplify by real-time PCR (RT-PCR) a region in the M gene (nt 26488 to 27784), from which an MluI restriction site was removed from the rSARS-CoV-2 cDNA via a silent mutation (FIG. 1B). As expected, the RT-PCR product from SARS-CoV-2-infected cells digested with MluI yielded two fragments with sizes of 351 and 946 bp (FIG. 3A, bottom). In contrast, the RT-PCR product from rSARS-CoV-2-infected cells was not digested with MluI (FIG. 3A, bottom). FIG. 3A is a photographic representation of undigested (top) and digested (bottom) samples as analyzed on an agarose gel. Vero E6 cells were mock infected or infected (MOI, 0.01) with rSARS-CoV-2 or the SARS-CoV-2 USA-WA1/2020 natural isolate. At 24 h post infection, total RNA from Vero E6 cells was extracted and a 1,297-bp region of the M gene (nt 26488 to 27784) was amplified by RT-PCR. Amplified DNA was subjected to MluI digestion (FIGS. 1A-1C). Undigested (top) and digested (bottom) samples were separated in a 0.7% agarose gel. The RT-PCR-amplified DNA product was also sequenced to verify the presence of the silent mutation in the MluI restriction site introduced in the viral genome of the rSARS-CoV-2 (FIGS. 1A-1C). The mutation introduced into the MluI restriction site in the rSARS-CoV-2 strain was confirmed by Sanger sequencing (FIG. 3B). FIG. 3B is an illustration of the MluI restriction site (underlined in red) in the SARS-CoV-2 construct, and the silent mutation introduced in the rSARS-CoV-2 construct to remove the MluI restriction site (T to A) is shown in the black box.

To further characterize the genetic identity of rSARS-CoV-2, next generation sequencing (NGS) was used to determine the complete genome sequence of the natural SARS-CoV-2 isolate from BEI Resources and the rescued rSARS-CoV-2 RNA, as well as the BAC plasmid used to rescue rSARS-CoV-2. About 4.95 million, 5.79 million, and 5.44 million reads were examined for the natural virus isolate, BAC plasmid, and rescued rSARS-CoV-2 RNA, resulting in coverages of 978×, 15,296×, and 1,944× per sample, respectively. Introduced variants were not present in the SARS-CoV-2 RNA but were effectively fixed in the BAC plasmid and rSARS-CoV-2. The presence of the genetic markers was confirmed at positions 21895 (S) and 26843 (M) in both the BAC plasmid and rSARS-CoV-2 (allele frequencies, >99.9%) (FIG. 3C, middle and bottom). FIG. 3C is a representation of verification of SARS-CoV-2 sequence (top panel), rSARS-CoV-2 sequence (middle panel), and SARS-CoV-2 BAC sequence (bottom panel). Non-reference alleles present in less than 1% of reads are not shown. The SARS-CoV-2 non-reference allele frequency was calculated by comparing short reads to the reference genome of the USA-WA1/2020 reference. All variants were at low frequency in the P6 natural isolate (top), the BAC (bottom), and rSARS-CoV-2 (middle), with the exception of introduced variants at positions 21895 and 26843, which were fixed in the BAC and in rSARS-CoV-2. Non-reference alleles present in less than 1% of reads are not shown.

Next, rSARS-CoV-2 and the natural isolate of SARS-CoV-2 were compared with respect to their growth properties in Vero E6 cells. Both rSARS-CoV-2 and SARS-CoV-2 made uniform plaques of similar sizes (FIG. 3D). FIG. 3D is a photographic representation of the plaque phenotype. Vero E6 cells were infected with 20 PFU of rSARS-CoV-2 (left) or the natural SARS-CoV-2 isolate (right). After 72 h of incubation at 37° C., cells were fixed and immunostained with the N protein 1C7 monoclonal antibody. Likewise, both rSARS-CoV-2 and SARS-CoV-2 exhibited similar growth kinetics and peak titers (FIG. 3E). FIG. 3E is a graphical representation of the growth kinetics of Vero E6 cells infected (MOI, 0.01) with rSARS-CoV-2 or the natural SARS-CoV-2 isolate. At the indicated times post-infection, tissue culture supernatants were collected and viral titers were assessed by plaque assay (PFU/ml). Data are presented as means SDs. LOD, limit of detection. These results confirmed the genetic identity of rSARS-CoV-2 and its ability to replicate to the same extent as the SARS-CoV-2 natural isolate in Vero E6 cells.

### Example 3

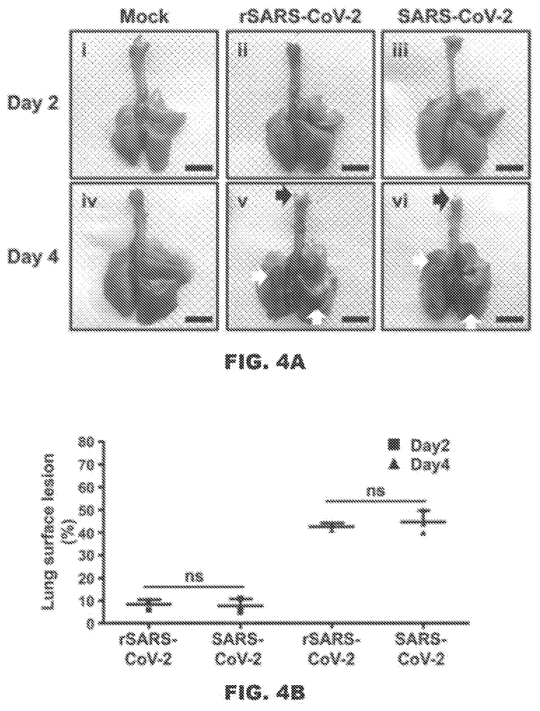

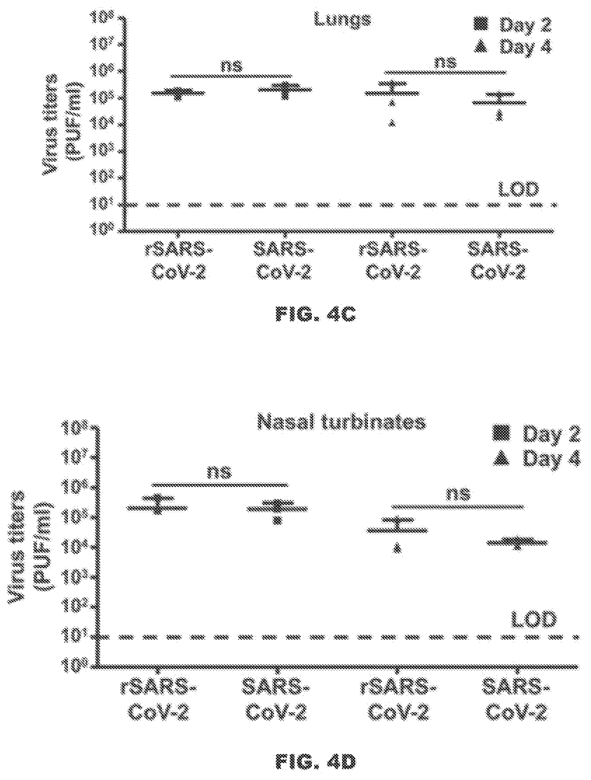

Pathogenicity of rSARS-CoV-2 in vivo. Golden Syrian hamsters (Mesocricetus auratus) have been shown to be a good rodent animal model for investigating the replication, virulence, and pathogenicity of both SARS-CoV and SARS-CoV-2 in vivo. To confirm that the rSARS-CoV-2 RNA generated using BAC-based reverse genetics exhibited the same replication capability, virulence, and pathogenicity as the natural SARS-CoV-2 isolate in vivo, golden Syrian hamsters were infected intranasally with 2×10⁴ PFU of either rSARS-CoV-2 or the natural SARS-CoV-2 isolate. Animals were euthanized at 2 and 4 days post infection, and lungs from mock-infected (FIG. 4Ai and FIG. 4Aiv) or infected (rSARS-CoV-2 (FIG. 4Aii and FIG. 4Av) and SARS-CoV-2 (FIG. 4Aiii and FIG. 4Avi) animals were observed for gross pathological changes, including congestion and atelectasis (white arrows) and frothy trachea exudates (black arrows). Scale bars, 1 cm. FIG. 4A is a set of photographs of gross pathological lung lesions to demonstrate the pathogenicity of rescued rSARS-CoV-2 in vivo. Mild multifocal congestion and consolidation were observed in 5 to 10% of the surfaces of lungs from rSARS-CoV-2 (FIG. 4Aii) and SARS-CoV-2 (FIG. 4Aiii)-infected animals at day 2 post infection. As expected, the gross pathological lesions were pronounced at day 4 post infection, with severe multifocal to locally extensive congestion and consolidation (white arrows) in 40 to 50% of the surfaces of the lungs (FIG. 4Av and vi). These lesions were widely distributed, covering both the right (cranial, medial, and caudal lobes) and the left lobes of the lungs. Particularly, the presence of frothy exudate (black arrows) in the tracheas of hamsters infected with either rSARS-CoV-2 or SARS-CoV-2 on day 4 post infection indicates an ongoing bronchopneumonia. No significant differences were observed in pathological lesions in the lungs at both days post infection between animals infected with rSARS-CoV-2 or SARS-CoV-2 (FIG. 4B). FIG. 4B is a graphical representation of the macroscopic pathology scoring analysis of lungs from rSARS-CoV-2 and SARS-CoV-2 animals at Day 2 and Day 4. Distributions of pathological lesions, including consolidation, congestion, and atelectasis, were measured using ImageJ and are represented as percentages of the total lung surface area. ns, not significant. (C and D) Virus titers. FIG. 4C and FIG. 4D are graphical representations of the viral titers in the lungs and nasal turbinates, respectively, of rSARS-CoV-2- and SARS-CoV-2-infected golden Syrian hamsters as evaluated at days 2 and 4 post infection (3 hamsters per time point). Data are means SDs. ns, not significant. Both rSARS-CoV-2 and SARS-CoV-2 replicated to similar levels in the lungs (FIG. 4C) and the nasal turbinates (FIG. 4D) of infected animals at days 2 and 4 post infection, indicating that the genetically engineered rSARS-CoV-2 clone replicates to levels comparable to those of the natural isolate in vivo. The rSARS-CoV-2 constructs were compared to the natural SARS-CoV-2 isolate in evaluation of their morbidity, mortality and viral replication profiles in the K18 human angiotensin converting enzyme 2 (hACE2) transgenic mice. The rSARS-CoV-2 constructs had similar morbidity, mortality and viral replication as compared to the natural isolate in this mouse model of SARS-CoV-2 infection.

### Example 4

Generation of rSARS-CoV-2 deficient mutant viruses. An embodiment includes a full-length infectious clone of the SARS-CoV-2 USA-WA1/2020 strain based on a BAC. The full-length cDNA copy of SARS-CoV-2 USA-WA1/2020 was sequentially assembled downstream of a cytomegalovirus (CMV) promoter into the pBeloBAC11 plasmid using synthetic fragments. After delivery of the BAC into host cells, the CMV promoter initiates the production of viral RNA from the nuclei of transfected cells by cellular RNA polymerase II. In an embodiment, the CMV promoter can be used to generate rSARS-CoV-2 from other cell lines. For example, rSARS-CoV-2 using the BAC-based constructs were rescued from human 293T and HeLa cells constitutively expressing human angiotensin-converting enzyme 2 (hACE2) (data not shown). The genetic identity of the rescued rSARS-CoV-2 clone was confirmed by sequencing. Notably, the rSARS-CoV-2 clone replicated in Vero E6 cells to levels comparable to those of the natural isolate as determined by growth kinetics and plaque assay. Importantly, using the golden Syrian hamster model of SARS-CoV-2 infection, both rSARS-CoV-2 and the natural SARS-CoV-2 isolate were demonstrated to have similar pathogenicity and growth capabilities in the upper and lower respiratory tracts of infected animals. Table 1 and FIGS. 11A-11C present several other recombinant constructs that have generated.

TABLE 1

| Type | Name |
|---|---|
| Wild-type virus | rSARS-CoV-2/WT |
| Reporter virus | rSARS-CoV-2/Δ7a-Venus |
| (FIG. 11A) | rSARS-CoV-2/Δ7a-mCherry |
| | rSARS-CoV-2/Δ7a-Nluc |
| | rSARS-CoV-2/ORF10-Venus |
| | rSARS-CoV-2/Venus-P2A-N |
| Deficient virus | rSARS-CoV-2/ΔS |
| (FIG. 11B) | rSARS-CoV-2/Δ3a |
| | rSARS-CoV-2/ΔE |
| | rSARS-CoV-2/Δ6 |
| | rSARS-CoV-2/Δ7a |
| | rSARS-CoV-2/Δ7b |
| | rSARS-CoV-2/Δ8 |
| | rSARS-CoV-2/Δ10 |
| Spike mutant virus | rSARS-CoV-2/Bristol deletion |
| (FIG. 11C) | rSARS-CoV-2/Furin deletion |
| | rSARS-CoV-2/D614G |

The pBeloBAC11 plasmid encoding the full-length viral genome of SARS-CoV-2 was used as the starting point. To generate the deficient rSARS-CoV-2, each of the viral open reading frames for the S, ORF3a, E, 6, 7a, 7b, 8 and 10 were deleted in the pBeloBAC11 plasmid encoding the remaining viral genome to produce pBeloBAC11-SARS-CoV-2-delS, -del3a, -delE, -del6, -del7a, -del7b, -del8 or -del10 plasmids for viral rescues (FIG. 11B). The BAC-based reverse genetics method as disclosed here was used to rescue rSARS-CoV-2-delS, -del3a, -delE, -del6, -del7a, -del7b, -del8 or -del10 viruses (FIG. 11B). FIG. 11B is a schematic representation of the rSARS-CoV-2-delS, -del3a, -delE, -del6, -del7a, -del7b, -del8 or -del10 constructs.

Generation of rSARS-CoV-2 spike mutant viruses. The pBeloBAC11 plasmid encoding the full-length viral genome of SARS-CoV-2 was used as the starting point. To generate the spike mutant rSARS-CoV-2, mutations in the spike protein were introduced in a shuttle vector and then subcloned into the pBeloBAC11 plasmid encoding the remaining viral genome to produce pBeloBAC11-SARS-CoV spike mutant plasmids for viral rescues (FIG. 11C). The BAC-based reverse genetics approach as disclosed here was used to rescue rSARS-CoV-2 spike mutants (FIG. 11C). FIG. 11C is a schematic representation of the rSARS-CoV-2 spike mutant virus constructs.

### Example 5

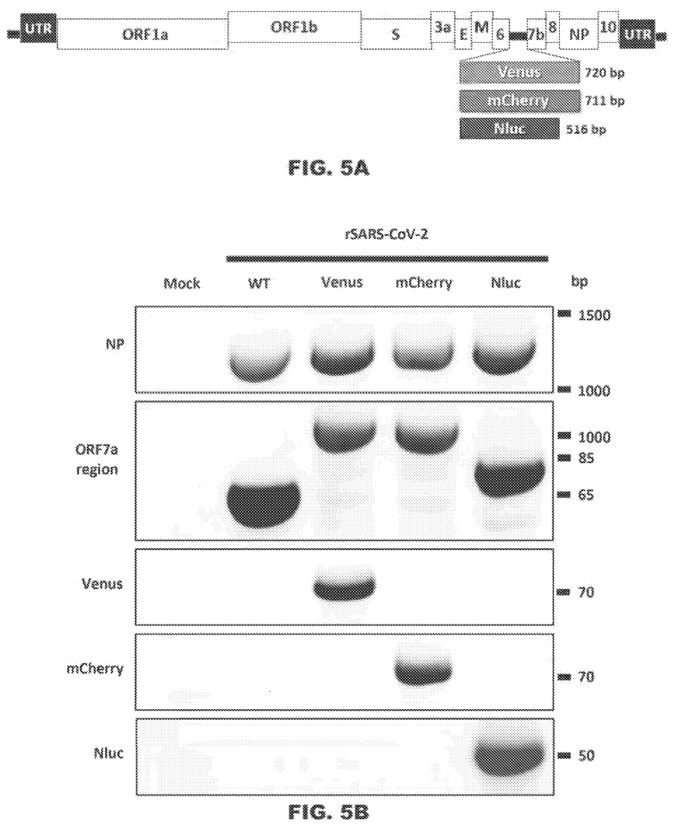

Generation of rSARS-CoV-2 expressing reporter genes. The pBeloBAC11 plasmid encoding the full-length viral genome of SARS-CoV-2 was used as the starting point. To generate the reporter-expressing rSARS-CoV-2, the 7a open reading frame (ORF) was substituted with Venus, mCherry, or Nluc gene in the pBeloBAC11 plasmid encoding the remaining viral genome to produce pBeloBAC11-SARS-CoV-2-del7a/Venus, -del7a/mCherry, or -del7a/Nluc plasmids for viral rescues (FIG. 11A). The BAC-based reverse genetics approach as disclosed here was used to rescue rSARS-CoV-2-Venus, -mCherry, and -Nluc reporter viruses (FIG. 5A). FIG. 5A is a schematic representation of the rSARS-CoV-2 constructs with Venus, mCherry, or Nluc genes. The rescue of rSARS-CoV-2 expressing -Venus, -mCherry, or -Nluc reporter genes were confirmed by RT-PCR using total RNA from mock-, rSARS-CoV-2/WT- or rSARS-CoV-2 reporter virus-infected cells using primers specific for the viral NP, the ORF7a region, or the individual reporter genes (FIG. 5B). As expected, primers specific for SARS-CoV-2 NP amplified a band of ˜1260 bp from the RNA extracted from rSARS-CoV-2-infected but not mock-infected cells. Amplified bands using primers in the ORF7a region resulted in the expected ˜566 bp in cells infected with rSARS-CoV-2/WT and ˜ 920, 911, and 815 bp in the case of cells infected with rSARS-CoV-2-Venus, -mCherry and -Nluc, respectively, based on the different size of the reporter genes. Primers specific for the reporter genes only results in the RT-PCR amplification of bands from cells infected with the respective reporter-expressing rSARS-CoV-2. These results demonstrate that substitution of the viral ORF7a for Venus, mCherry, or Nluc genes results in the successful recovery of rSARS-CoV-2 viruses containing these reporter genes.

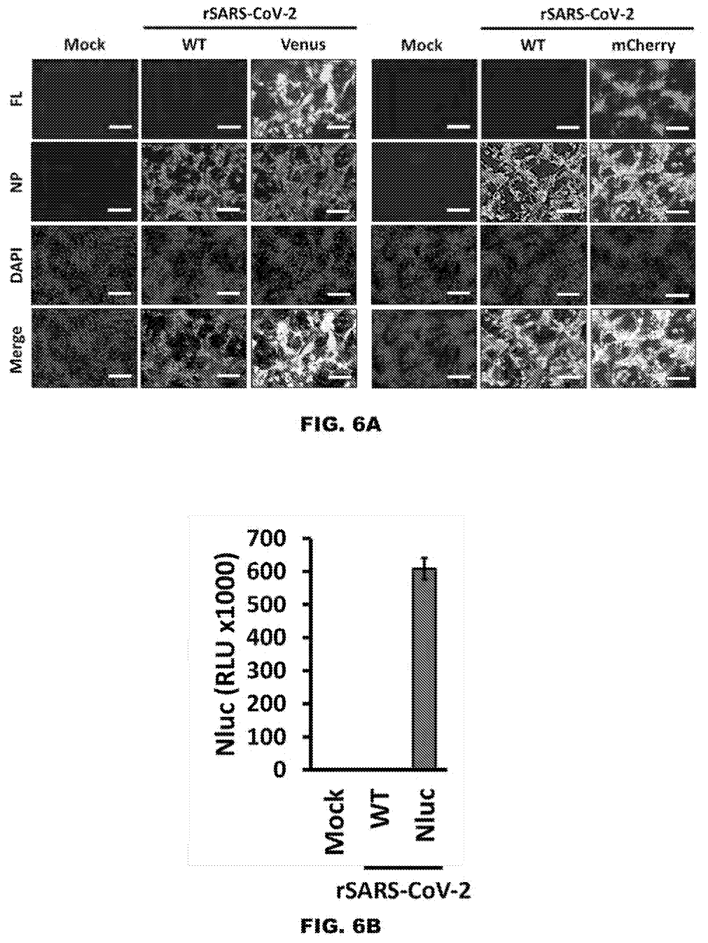

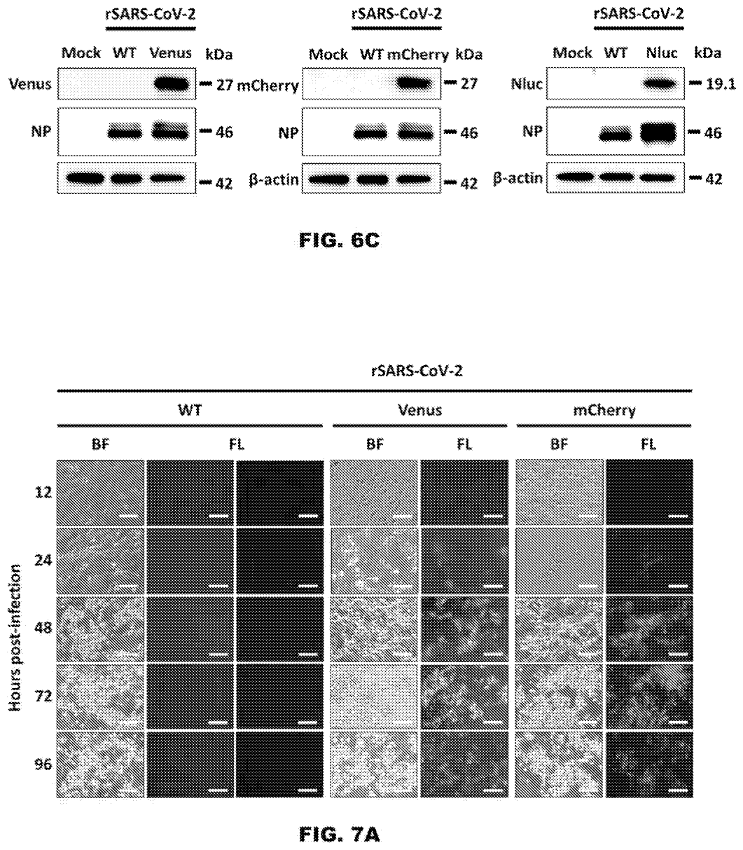

Characterization of rSARS-CoV-2 expressing reporter genes. Next, the reporter-expressing rSARS-CoV-2 were characterized by evaluating the expression levels of Venus, mCherry, or Nluc in cell cultures, and compared them to those of cells infected with rSARS-CoV-2/WT (FIGS. 6A-6C). The rSARS-CoV-2 expressing Venus and mCherry were directly visualized under a fluorescence microscope (FIG. 6A). Indirect immunofluorescence microscopy using a MAb against SARS-CoV NP was used to detect rSARS-CoV-2/WT infection (FIG. 6A). As expected, Venus or mCherry expression were only observed in Vero E6 cells infected with rSARS-CoV-2 expressing Venus or mCherry, respectively, but not in cells infected with rSARS-CoV-2/WT (FIG. 6A). Importantly, only cells infected with rSARS-CoV-2-Venus or rSARS-CoV-2-mCherry were detected using green or red filters, respectively (data not shown). As expected, the viral NP was detected in cells infected with rSARS-CoV-2-WT, -Venus, or -mCherry viruses (FIG. 6A). Expression of Nluc in rSARS-CoV-2-Nluc-infected cells was evaluated from tissue culture supernatants at 48 h post-infection (FIG. 6B). High levels of Nluc expression were detected in culture supernatants of cells infected with rSARS-CoV-2-Nluc but not from mock or rSARS-CoV-2/WT infected cells (FIG. 6B). These results demonstrate that Vero E6 cells infected with rSARS-CoV-2-Venus, -mCherry, or -Nluc expresses the corresponding reporter genes and that viral infections can be detected by fluorescence (rSARS-CoV-2-Venus or -mCherry) or luciferase (rSARS-CoV-2-Nluc) without the need of antibodies that were required for the detection of rSARS-CoV-2/WT. Reporter protein expression levels were evaluated by Western blot assay using cell lysates from either mock, rSARS-CoV-2-WT, or rSARS-CoV-2-Venus, -mCherry, or -Nluc infected cells using MAbs against the viral NP, the reporter genes, or actin as a loading control (FIG. 6C). As expected, reporter gene expression was detected in cell lysates of cells infected with the respective reporter-expressing rSARS-CoV-2 but not from mock or rSARS-CoV-2-WT infected cells. Viral NP expression was detected in cell lysates from all virus-infected cells, but not mock-infected cells (FIG. 6C).

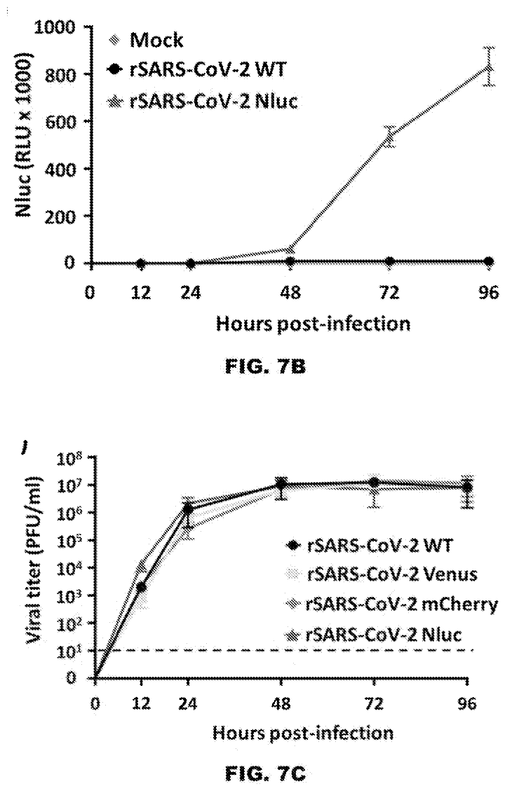

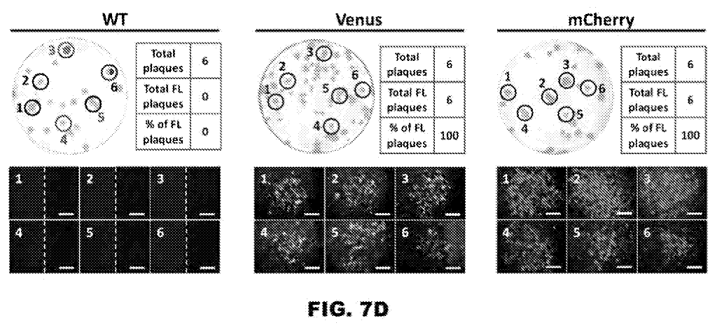

Reporter gene expression was assessed over a period of 96 h in cells that were mock-infected (data not shown) or cells infected with WT or reporter-expressing rSARS-CoV-2 (FIGS. 7A-7D). Venus and mCherry expression levels were determined using fluorescence microscope (FIG. 7A), while Nluc activity in tissue culture supernatants from infected cells was detected using a luminometer (FIG. 7B). Venus and mCherry expression were detected as early as 24 h post-infection and fluorescent protein expression increased over time until 96 h post-infection where a decrease in fluorescence was observed because of CPE caused by viral infection (brightfield, BF). Similar CPE, but not fluorescent expression, was also observed in cells infected with rSARS-CoV-2/WT (FIG. 7A). Levels of Nluc expression were also detected as early as 24 h post-infection and increase in a time-dependent matter (FIG. 7B).

To assess whether deletion of 7a ORF and insertion of reporter genes compromised viral fitness in cultured cells, growth kinetics of reporter-expressing rSARS-CoV-2 was compared to those of rSARS-CoV-2/WT (FIG. 7C). All the reporter-expressing rSARS-CoV-2 exhibited similar growth kinetics and peak viral titers of infection to that of rSARS-CoV-2/WT (FIG. 7C), suggesting that deletion of the 7a ORF and insertion of the reporter genes did not significantly affect viral fitness, at least in cultured cells. These results also support previous findings with SARS-CoV where deletion of the 7a ORF and insertion of reporter genes did not impact viral fitness in vitro. These results were further confirmed when the plaque phenotype of the rSARS-CoV-2 expressing fluorescent reporter genes were evaluated and compared to those of rSARS-CoV-2/WT (FIG. 7D). Similar plaque sizes were observed in Vero E6 cells infected with rSARS-CoV-2/WT and rSARS-CoV-2 expressing Venus or mCherry (FIG. 7D). Notably, Venus-positive or mCherry-positive plaques were only detected in cells infected with rSARS-CoV-2-Venus or -mCherry, respectively, and not in rSARS-CoV-2/WT infected cells (FIG. 7D). Importantly, fluorescent plaques overlapped with those detected by immunostaining using the SARS-CoV NP 1C7 MAb. Similar to the growth kinetics data, no significant differences were found in the plaque size of reporter-expressing rSARS-CoV-2 compared to rSARS-CoV-2/WT (FIG. 7D).

### Example 6

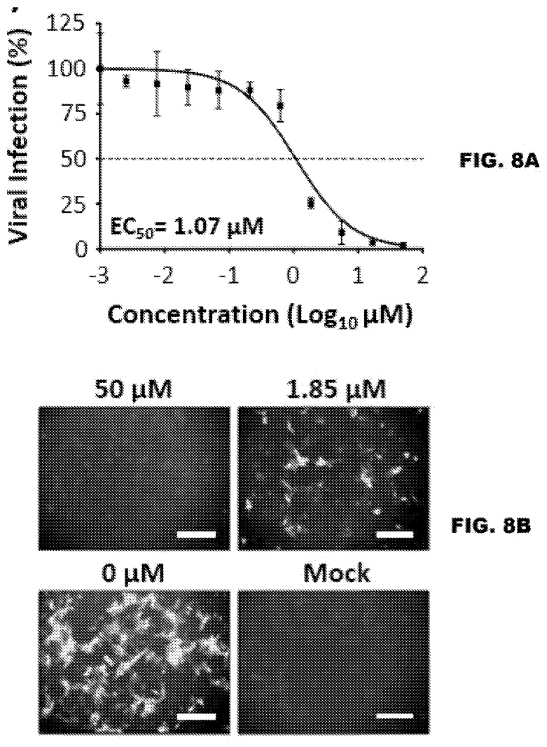

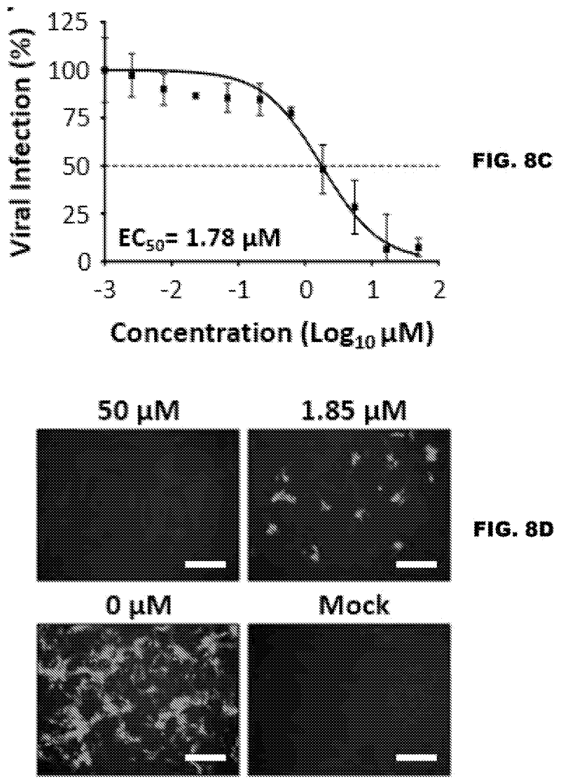

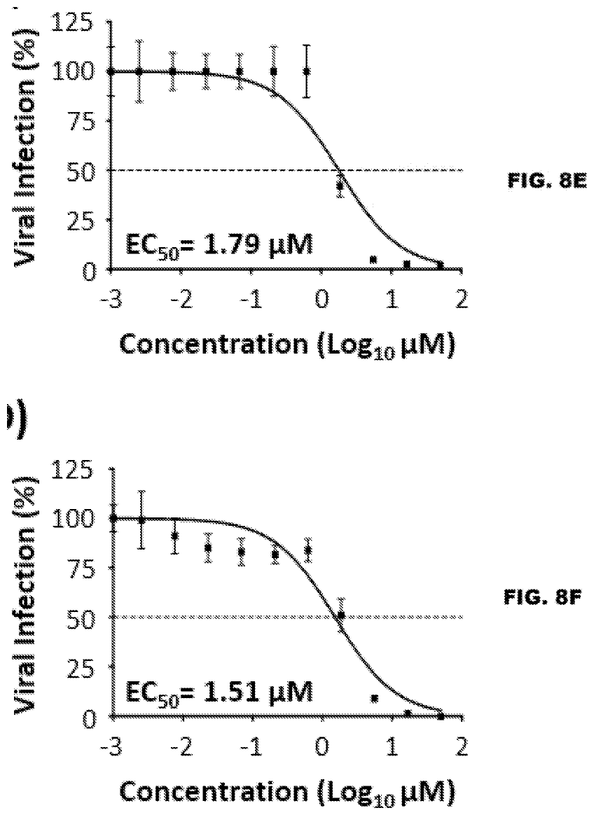

A reporter-based microneutralization assay for the identification of antivirals. To determine the feasibility of using the reporter-expressing rSARS-CoV-2 for the identification of antivirals, the ability of Remdesivir to inhibit SARS-CoV-2 in reporter-based microneutralization assays was evaluated (FIGS. 8A-8F). Remdesivir has been previously described to inhibit SARS-CoV-2 infection and is the only FDA-approved antiviral for the treatment of SARS-CoV-2. The EC₅₀ of Remdesivir against rSARS-CoV-2-Venus (FIG. 8A, 1.07 μM), -mCherry (FIG. 8C, 1.78 μM), or Nluc (FIG. 8E, 1.79 μM) were similar to those obtained with rSARS-CoV-2/WT (FIG. 8F, 1.51 μM). Immunohistochemistry analysis of the reporter-expressing rSARS-CoV-2 assays are presented in FIGS. 8B and 8D for rSARS-CoV-2-Venus and -mCherry, respectively. This demonstrates the feasibility of using these reporter-expressing rSARS-CoV-2 compositions and the reporter-based assay to easily identify compounds with antiviral activity based on fluorescent or luciferase expression and without the need of MAbs to detect the presence of the virus in infected cells.

### Example 7

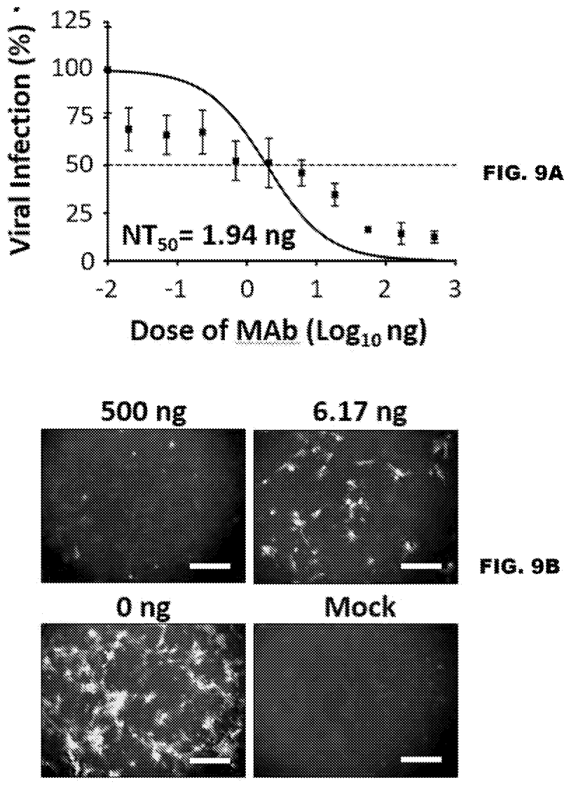

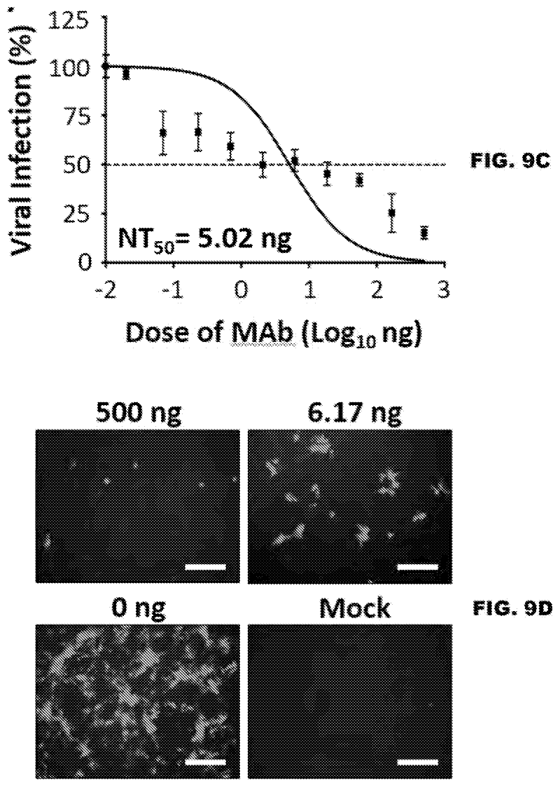

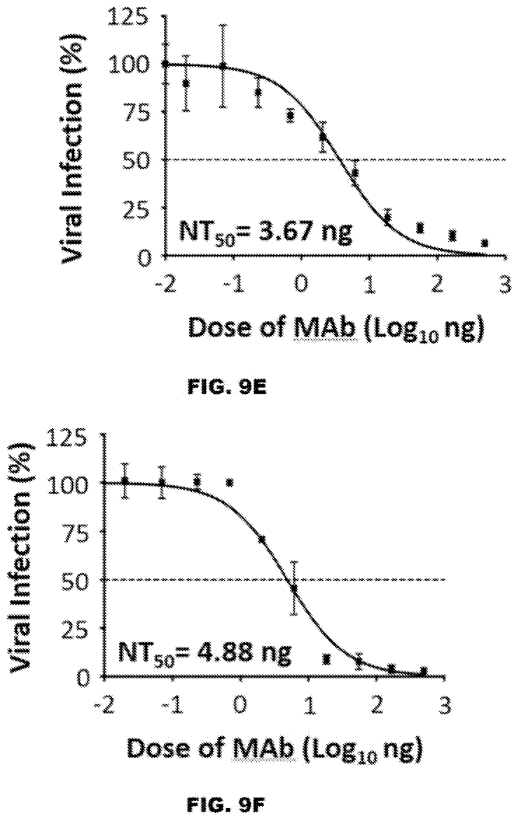

A reporter-based microneutralization assay for the identification of Nabs. The reporter-expressing rSARS-CoV constructs can be used in reporter-based microneutralization assays to identify NAbs against SARS-CoV-2. Human MAb (1212C2), which binds and neutralizes SARS-CoV-2 infection both in vitro and in vivo, was used. The NT₅₀ of 1212C2 against rSARS-CoV-2-Venus (FIG. 9A, 1.94 ng), -mCherry (FIG. 9C, 5.02 ng), or Nluc (FIG. 9E, 3.67 ng) were similar to those observed with rSARS-CoV-2/WT (FIG. 9F, 4.88 ng). Immunohistochemistry analysis of the reporter-expressing rSARS-CoV-2 assays are presented in FIGS. 9B and 9D for rSARS-CoV-2-Venus and -mCherry, respectively.

### Example 8

Genetic stability of rSARS-CoV-2 in vitro. The genetic stability of reporter-expressing recombinant viruses is important to demonstrate their viability in in vitro and/or in vivo studies. To evaluate the ability of the rSARS-CoV-2 compositions to maintain fluorescent reporter gene expression, viruses were consecutively passaged in Vero E6 cells and Venus and mCherry expression were determined by plaque assay using fluorescent microscopy (FIGS. 10A and 10B). To that end, fluorescent expression of over 40 plaques was evaluated before immunostaining with an anti-SARS-CoV NP MAb 1C7. The Venus and mCherry fluorescent expression from the rSARS-CoV-2 construct was genetically stable with nearly 100% of the plaques analyzed under a fluorescent microscope (FIG. 10A). The complete genome sequences of the reporter-expressing rSARS-CoV-2 at a particular passage (P3) with those of additional passages (P4 and P5) were evaluated using NGS (FIG. 10B). In the case of rSARS-CoV-2/Venus (FIG. 10B, top panel), few variants were found at low frequencies after two additional passages (P5), indicating no significant changes and/or deletions in the viral genome. However, for rSARS-CoV-2/mCherry (FIG. 10B, middle panel), variants containing mutations at positions 21784 and 24134 were found in the viral stock (P3) and the frequency of these mutations increased after additional passages (P4 and P5). In the case of rSARS-CoV-2/Nluc (FIG. 10B, lower panel), a mutation at position 24,755 was found in the viral stock (P3). Frequency of this mutation increased up to 100% after 2 additional passage (P5). Other, less abundant, mutations at positions 13419, 23525, and 26256 were also found after the additional 2 passages (P5) (FIG. 10B, lower panel). These mutations are most likely due to viral adaptation to Vero E6 cells, but as different mutations were found in the three reporter-expressing rSARS-CoV-2, these mutations may be related to the nature of the reporter gene. These results indicate that the reporter-expressing rSARS-CoV-2 are genetically stable in Vero E6 cells.

### Example 9

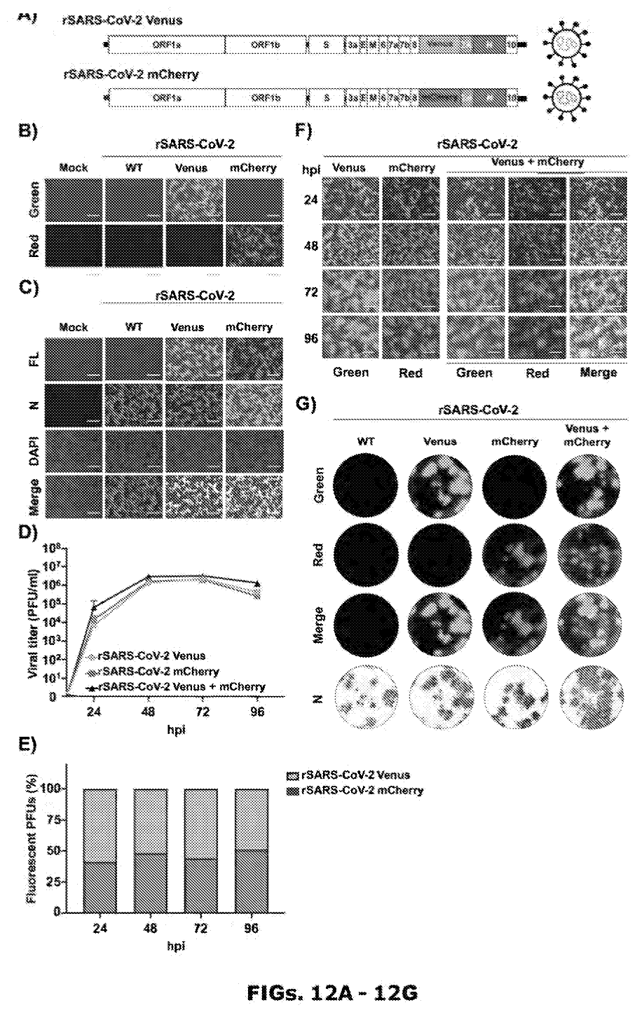

Generation and characterization of rSARS-CoV-2 expressing FPs. The pBeloBAC 11 plasmid encoding the full-length viral genome of SARS-CoV-2 WA-1 was used as backbone to generate the different rSARS-CoV-2. New rSARS-CoV-2 reporter viruses were constructed that retained all viral genes—the Venus or mCherry FP was cloned upstream of the viral N gene using the PTV-1 2A autocleavage sequence (FIG. 12A). Recombinant viruses expressing FPs using this experimental approach based on the use of the 2A cleavage site from the N locus do not require removing any viral genes, express higher levels of reporter gene expression compared to those previously described from the locus of the ORF7a, and are genetically more stable. To characterize the newly generated FP-expressing rSARS-CoV-2, the expression levels of Venus and mCherry were first assessed. Confluent monolayers of Vero E6 cells were infected (MOI 0.01) with either rSARS-CoV-2 WT, rSARS-CoV-2 Venus, rSARS-CoV-2 mCherry, or mock-infected, and then visualized by fluorescence microscopy (FIG. 12B). As expected, only cells infected with rSARS-CoV-2 Venus or rSARS-CoV-2 mCherry were detected under a fluorescent microscope (FIG. 12B). Cells infected with rSARS-CoV-2 WT, rSARS-CoV-2 Venus, and rSARS-CoV-2 mCherry showed comparable levels of N protein expression (FIG. 12C). The multi-step growth kinetics of the newly generated rSARS-CoV-2 was examined. Vero E6 cells were infected (MOI 0.01) with rSARS-CoV-2 Venus or rSARS-CoV-2 mCherry, individually or together, and tissue culture supernatants collected over a course of 96 hours to determine viral titers (FIG. 12D). Kinetics of production and peak titers of infectious progeny were similar for rSARS-CoV-2 expressing Venus or mCherry. Results from co-infection experiments using Venus- and mCherry-expressing rSARS-CoV-2 indicated that both viruses had similar fitness under the experimental conditions used (FIG. 12E). These results were further validated by assessing FP expression in cells infected with rSARS-CoV-2 Venus and rSARS-CoV-2 mCherry, alone or in combination (FIG. 12F). Moreover, both rSARS-CoV-2 Venus and rSARS-CoV-2 mCherry exhibited similar plaque formation efficiency and plaque size phenotype as the parental rSARS-CoV-2 WT (FIG. 12G).

FIGS. 12A-12G are directed to the generation and characterization of Venus and mCherry-expressing rSARS-CoV-2. FIG. 12A is a diagrammatic representation of Venus and mCherry rSARS-CoV-2. Reporter genes Venus (green) or mCherry (red) were inserted upstream of the N protein (dark blue), flanked by the PTV-1 2A autocleavage sequence (light blue). FIGS. 12B and 12C are sets of photographic images of Venus and mCherry expression from rSARS-CoV-2. Vero E6 cells (6-well plate format, 10⁶ cells/well, triplicates) were mock-infected or infected (MOI 0.01) with rSARS-CoV-2 Venus or rSARS-CoV-2 mCherry (FIG. 12B). At 24 hpi, cells were fixed in 10% neutral buffered formalin and visualized under a fluorescence microscope for Venus or mCherry expression. A cross-reactive mMAb against SARS-CoV N protein (1C7C7) was used for staining of infected cells (FIG. 12C). DAPI was used for nuclear staining. FL: fluorescent field. FIGS. 12D and 12E are graphical representations of the multi-step growth kinetics. Vero E6 cells (6-well plate format, 10⁶ cells/well, triplicates) were mock-infected or infected (MOI 0.01) with rSARS-CoV-2 Venus and rSARS-CoV-2 mCherry, alone or together, and tissue cultured supernatants were collected at the indicated times p.i. to assess viral titers using standard plaque assay (FIG. 12D). The amount of Venus- and/or mCherry-positive rSARS-CoV-2 at the same times p.i. in cells infected with both viruses were also determined using plaque assay (FIG. 12E). FIG. 12F is a set of photographic images of Vero E6 cells expressing rSARS-CoV-2 Venus or rSARS-CoV-2 mCherry, individually and in combination, and then visualized by fluorescence microscopy at four time points. Images of infected cells under a fluorescent microscope at the same times p.i. are shown. FIG. 12G is a set of photographic images of plaque assays using rSARS-CoV-2 WT, rSARS-CoV-2 Venus, rSARS-CoV-2 mCherry, individually and in combination. Vero E6 cells (6-well plate format, 10⁶ cells/well, triplicates) were mock-infected or infected with -20 PFU of rSARS-CoV-2, rSARS-CoV-2 Venus, rSARS-CoV-2 mCherry, or both rSARS-CoV-2 Venus and rSARS-CoV-2 mCherry. At 72 hpi, fluorescent plaques were assessed using a Chemidoc instrument. Viral plaques were also immunostained with the SARS-CoV N protein 1C7C7 cross-reactive mMAb. Fluorescent green, red and merge imaged are shown. Viral plaques immunostained with the 1C7C7 SARS-CoV N protein cross-reactive mMAb are shown in the bottom. Representative images are shown for panels 12B, 12C and 12F and 12G. Scale bars=300 μm.

### Example 10

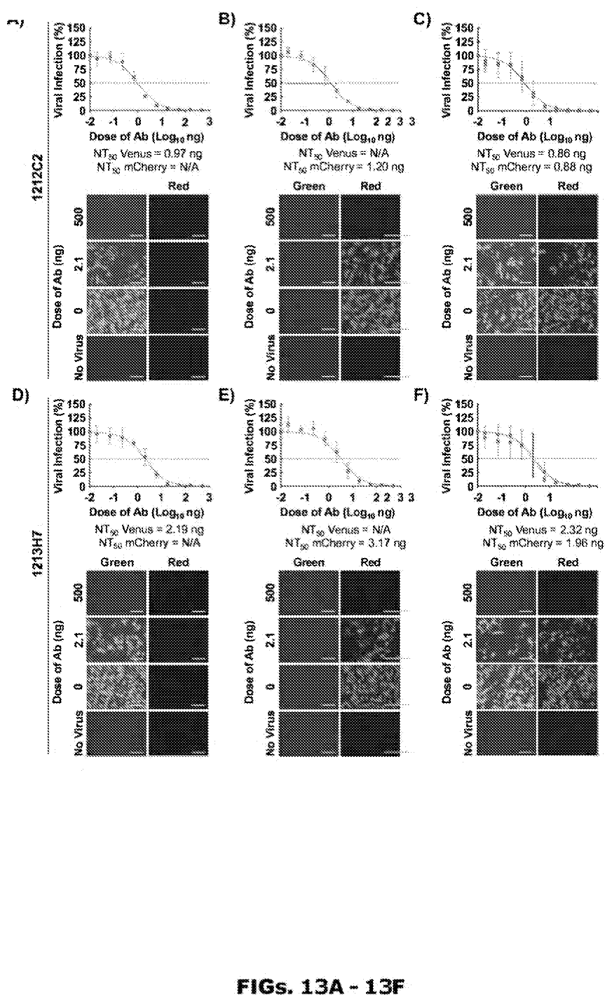

A bifluorescent-based assay for the identification of SARS-CoV-2 Nabs. The feasibility of using these two FP-expressing rSARS-CoV-2, alone and in combination, to identify NAbs against SARS-CoV-2 was assessed. The hMAbs 1212C2 and 1213H7 were used as both have been shown to potently neutralize rSARS-CoV-2. NT50 values of 1212C2 against rSARS-CoV-2 Venus (0.97 ng) (FIG. 13A), rSARS-CoV-2 mCherry (1.20 ng) (FIG. 13B), as well as rSARS-CoV-2 Venus and rSARS-CoV-2 mCherry together (0.86 ng and 0.88 ng, respectively) (FIG. 13C) were similar to those reported using a natural SARS-CoV-2 WA-1 isolate 16,22. NT50 of 1213H7 against rSARS-CoV-2 Venus (2.19 ng) (FIG. 13D), rSARS-CoV-2 mCherry (3.17 ng) (FIG. 13E), and both, rSARS-CoV-2 Venus and rSARS-CoV-2 mCherry together (2.32 ng and 1.96 ng, respectively) (FIG. 13F) were similar to those obtained with a SARS-CoV-2 WA-1 natural isolate 16. These results demonstrated the feasibility of using rSARS-CoV-2 expressing Venus and mCherry reporter genes in a new bifluorescent-based assay to identify SARS-CoV-2 NAbs.

FIGS. 13A-13F are sets of graphical representations and photographic images directed to the bifluorescent-based assay to identify Nabs. Confluent monolayers of Vero E6 cells (4×10⁴ cells/well, 96-plate well format, quadruplicates) were infected (MOI 0.1) with rSARS-CoV-2 Venus (FIGS. 13A and 13D), rSARS-CoV-2 mCherry (FIGS. 13B and 13E), o both rSARS-CoV-2 Venus and rSARS-CoV-2 mCherry (FIGS. 13C and 13F). After 1 h infection, p.i. media containing 3-fold serial dilutions of 1212C2 (FIGS. 13A-13C) or 1213H7 (FIGS. 13D-13F) hMAbs (starting concentration 500 ng) was added to the cells. At 48 hpi, cells were fixed with 10% neutral buffered formalin and levels of fluorescence expression were quantified in a fluorescent plate reader and analyzed using Gen5 data analysis software (BioTek). NT₅₀ values of 1212C2 and 1213H7 hMAbs for each virus, alone or in combination, were determined using GraphPad Prism. Dashed lines indicate 50% viral neutralization. Data are means and SD from quadruplicate wells. Representative images are shown. Scale bars=300 μm.

### Example 11

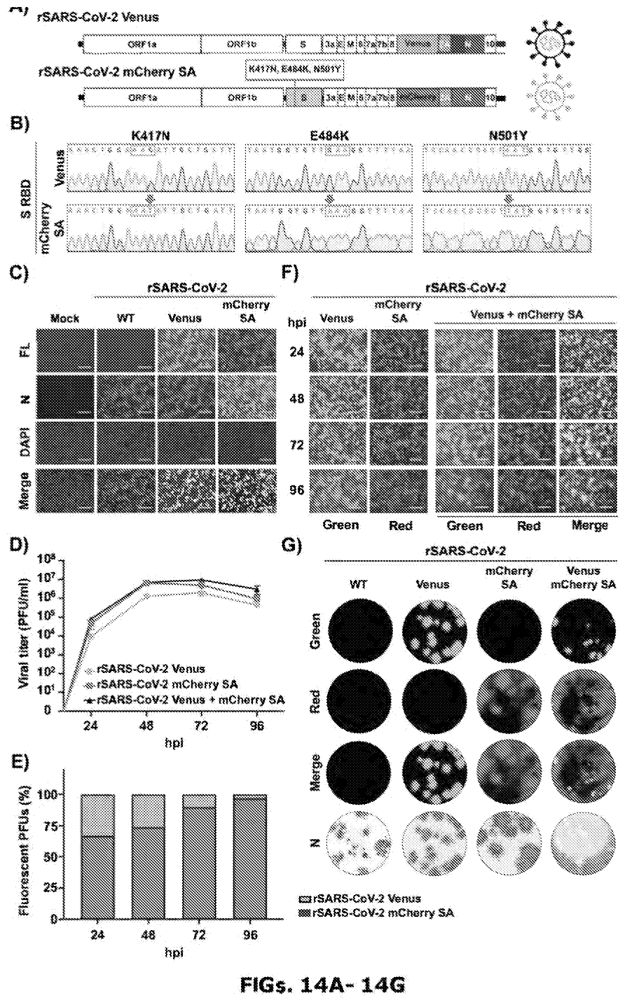

Generation and characterization of rSARS-CoV-2 mCherry SA. The emergence of new SARS-CoV-2 VoC, including the SA B.1.351 (β), is a major concern with the ongoing COVID-19 pandemic since the efficacy of current vaccines against recently identified VoC may be diminished. An assay was developed to evaluate the protective efficacy of hMAbs against WA-1 and SA VoC within the same well. For this, a rSARS-CoV-2 containing the K417N, E484K, and N501Y mutations found in the S RBD of the SA strain of SARS-CoV-2 and expressing also mCherry, referred to as rSARS-CoV-2 mCherry SA was generated (FIG. 14A). The genetic identity of the rescued rSARS-CoV-2 mCherry SA was confirmed by Sanger sequencing (FIG. 14B). The rSARS-CoV-2 mCherry SA was characterized by assessing reporter expression levels using rSARS-CoV-2 and rSARS-CoV-2 Venus as controls. Vero E6 cells were infected (MOI 0.01) with rSARS-CoV-2 WT, rSARS-CoV-2 Venus, or rSARS-CoV-2 mCherry SA, and expression of Venus and mCherry assessed by epifluorescent microscopy (FIG. 14C). Only cells infected with rSARS-CoV-2 Venus or rSARS-CoV-2 mCherry SA were fluorescent. However, immunostaining with the SARS-CoV cross-reactive N protein mMAb (1C7C7) detected cells infected with rSARS-CoV-2 WT, rSARS-CoV-2 Venus and rSARS-CoV-2 mCherry SA (FIG. 14C). Next, the growth kinetics of rSARS-CoV-2 mCherry SA and rSARS-CoV-2 Venus in Vero E6 cells was compared (FIGS. 14D-14F). Interestingly, at all hpi tested tissue culture supernatants from rSARS-CoV-2 mCherry SA infected cells had higher viral titers than those from rSARS-CoV-2 Venus infected cells (FIG. 14D), which correlated with a higher number of mCherry than Venus positive cells in cells co-infected with rSARS-CoV-2 Venus and rSARS-CoV-2 mCherry SA (FIGS. 14E and 14F). These results were further confirmed when multiplication of rSARS-CoV-2 Venus and rSARS-CoV-2 mCherry SA were assessed by plaque assay (FIG. 3G). Larger plaque foci were observed in cells infected with rSARS-CoV-2 mCherry SA compared to those infected with rSARS-CoV-2 Venus (FIG. 14G). A similar fitness advantage of a natural SARS-CoV-2 SA natural isolate was observed over SARS-CoV-2 WA-124 (data not shown).

FIGS. 14A-14G are directed to the generation and characterization of rSARS-CoV-2 mCherry SA. FIG. 14A is a schematic representation of rSARS-CoV-2 mCherry SA construct. The genome of a rSARS-CoV-2 Venus (top) and the rSARS-CoV-2 with the three mutations (K417N, E484K, and N501Y) present in the S RBD of the SA B.1.351 (beta, 3) VoC expressing mCherry (bottom) is shown. FIG. 14B is a set of images from the sequencing of rSARS-CoV-2 mCherry SA. Sanger sequencing results of the rSARS-CoV-2 Venus (top) and the rSARS-CoV-2 mCherry SA with the K417N, E484K, and N501Y substitutions in the RBD of the S glycoprotein (bottom) are indicated. FIG. 14C is a set of photographic images of reporter gene expression. Vero E6 cells (6-well plate format, 10⁶ cells/well, triplicates) were mock-infected or infected (MOI 0.01) with rSARS-CoV-2, rSARS-CoV-2 Venus, or rSARS-CoV-2 mCherry SA. Infected cells were fixed in 10% neutral buffered formalin at 24 hpi and visualized under a fluorescence microscope for Venus or mCherry expression. FIGS. 14D and 14E are graphical representations of the multi-step growth kinetics and FIG. 14F is the corresponding set of photographic images. Vero E6 cells (6-well plate format, 10⁶ cells/well, triplicates) were mock-infected or infected (MOI 0.01) with rSARS-CoV-2 Venus, rSARS-CoV-2 mCherry SA, or both rSARS-CoV-2 Venus and rSARS-COV-2 mCherry SA. Tissue cultured supernatants were collected at the indicated times p.i. to assess viral titers using standard plaque assay (FIG. 14D). The amount of Venus- and/or mCherry-positive plaques at the same times p.i. were determined using fluorescent microscopy (FIG. 14E). Images of infected cells under a fluorescent microscope at the same times p.i. are shown (FIG. 14F). FIG. 14G is a set of photographic images of the plaque assay. Vero E6 cells (6-well plate format, 10⁶ cells/well, triplicates) were mock-infected or infected with -20 PFU of rSARS-CoV-2, rSARS-CoV-2 Venus, rSARS-CoV-2 mCherry SA, or both rSARS-CoV-2 Venus and rSARS-CoV-2 mCherry SA. At 72 hpi, fluorescent plaques were assessed using a Chemidoc instrument. Viral plaques were also immunostained with the SARS-CoV N protein 1C7C7 cross-reactive mMAb. Fluorescent green, red and merge imaged as shown. Representative images are shown for panels C, F and G. Scale bars=300 μm.

### Example 12

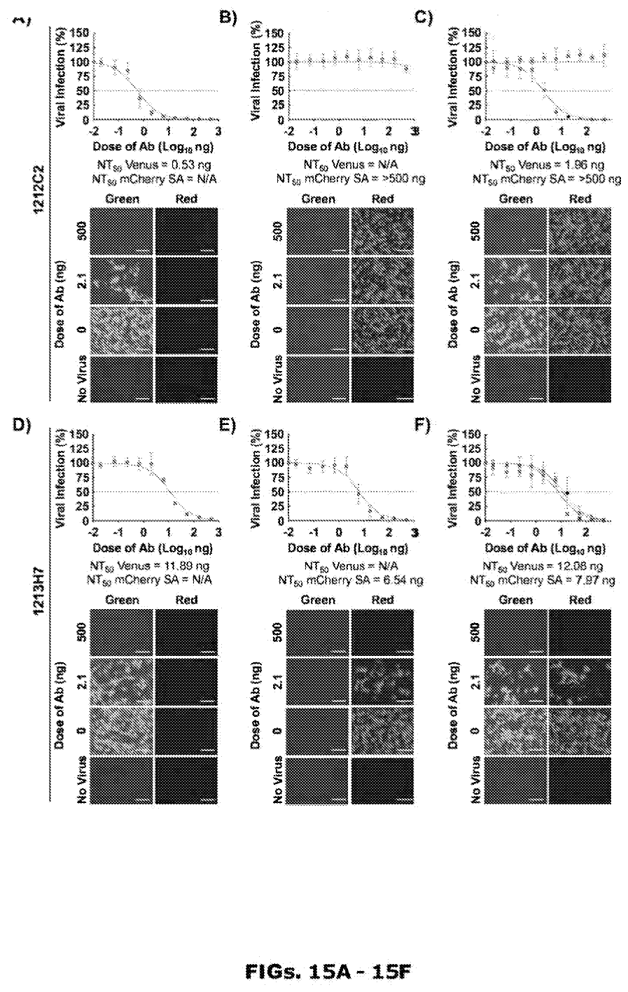

A bifluorescent-based assay to identify SARS-CoV-2 broadly Nabs. A bifluorescent-based assay using the rSARS-CoV-2 Venus and rSARS-CoV-2 mCherry SA was developed to identify broadly NAbs. To that end, the 1212C2 and 1213H7 hMAbs were used (FIGS. 13A-13F). Preliminary data using natural SARS-CoV-2 WA-1 and SA isolates showed that 1212C2 neutralized SARS-CoV-2 WA-1 but not SARS-CoV-2 SA VoC, while 1213H7 neutralized both viral isolates (data not shown). As expected, 1212C2 was able to efficiently neutralize rSARS-CoV-2 Venus (NT50 0.53 ng) (FIG. 15A) but not rSARS-CoV-2 mCherry SA (NT50>500 ng) (FIG. 15B), alone or in combination (NT50 1.96 ng and >500 ng, respectively) (FIG. 15C). In contrast, 1213H7 was able to efficiently neutralize both rSARS-CoV-2 Venus (NT50 11.89 ng) (FIG. 15D) and rSARS-CoV-2 mCherry SA (NT50 6.54 ng) (FIG. 15E), alone or in combination (NT50 12.08 and 7.97 ng, respectively) (FIG. 15F). Importantly, NT50 values observed with the FP-expressing rSARS-CoV-2, alone or in combination, were similar to those obtained with SARS-CoV-2 WA-1 and SA natural viral isolates (REF) and data not shown). These results demonstrated the feasibility of using this novel bifluorescent-based assay to readily and reliably identify hMAbs with neutralizing activity against both SARS-CoV-2 strains within the same assay and that results recapitulate those using individual viral infections and classical neutralization assays using natural viral isolates.

FIGS. 15A-15F are sets of graphical representations and photographic images directed to a bifluorescent-based assay to identify SARS-CoV-2 broadly Nabs. Confluent monolayers of Vero E6 cells (4×10⁴ cells/well, 96-plate well format, quadruplicates) were infected (MOI 0.1) with rSARS-CoV-2 Venus (FIGS. 15A and 15D), rSARS-CoV-2 mCherry SA (MOI 0.01) (FIGS. 15B and 15E), o both rSARS-CoV-2 Venus (MOI 0.1) and rSARS-CoV-2 mCherry SA (MOI 0.01) (FIGS. 15C and 15F). After 1 h infection, p.i. media containing 3-fold serial dilutions of 12C2C2 (FIGS. 15A-15C) or 1213H7 (FIGS. 15A-15C) hMAbs (starting concentration 500 ng) was added to the cells. At 48 hpi, cells were fixed with 10% neutral buffered formalin and levels of fluorescence expression were quantified in a fluorescent plate reader and analyzed using Gen5 data analysis software (BioTek). The NT₅₀ values of 1212C2 and 1213H7 hMAbs for each virus, alone or in combination, were determined using GraphPad Prism. Dashed lines indicate 50% viral neutralization. Data are means and SD from quadruplicate wells. Representative images are shown. Scale bars=300 μm.

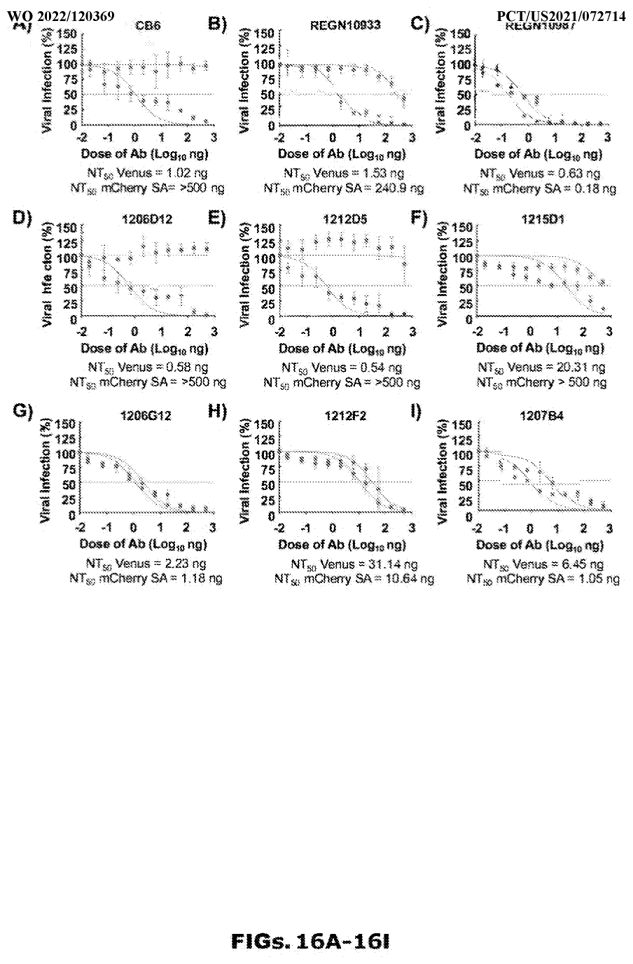

To further demonstrate the feasibility of this new bifluorescence-based assay to identify hMAbs and their ability to neutralize different SARS-CoV-2 strains present in the same sample the neutralizing activity of a selected set of previously described hMAbs was assessed. CB6, REGN10933, and REGN10987 hMAbs were used as internal controls in the assay. CB6 (FIG. 16A) and REGN10933 (FIG. 16B) neutralized rSARS-CoV-2 Venus (NT50 of 1.02 and 1.53 ng, respectively) but exhibited limited (REGN10933, NT50>240.9 ng) or none (CB6, NT50>500 ng) neutralization against rSARS-CoV-2 mCherry SA. On the other hand, REGN10987 (FIG. 16C) efficiently neutralized both rSARS-CoV-2 Venus and rSARS-CoV-2 mCherry SA (NT50 of 0.63 and 0.18 ng, respectively) (FIG. 16C). Some of the tested hMAbs were also able to specifically neutralize rSARS-CoV-2 Venus but not rSARS-CoV-2 mCherry SA, including 1206D12 (NT50 0.58 and >500 ng, respectively) (FIG. 16D) and 1212D5 (NT50 0.54 and >500 ng, respectively) (FIG. 16E). The hMAb 1215D1 efficiently neutralized rSARS-CoV-2 Venus but had reduced neutralization of rSARS-CoV-2 mCherry SA (NT50 1.08 and 91.3 ng, respectively) (FIG. 16F). Certain hMAbs with broadly neutralizing activity against both rSARS-CoV-2 Venus and rSARS-CoV-2 mCherry SA were identified, including 1206G12 (NT50 of 2.23 and 1.18 ng, respectively) (FIG. 16G), 1212F2 (NT50 of 31.14 and 10.64 ng, respectively) (FIG. 16H), and 1207B4 (6.45 and 1.05 ng, respectively) (FIG. 16I). Notably, NT50 values obtained in the bifluorescent-based assay were comparable to those obtained using individual natural viral isolates, further supporting the feasibility of this novel bifluorescent-based assay to identify broad neutralizing hMAbs against different SARS-CoV-2 strains.

FIGS. 16A-16I are graphical representations directed to bifluorescent-based assays for identification of SARS-CoV-2 broadly NAbs. Confluent monolayers of Vero E6 cells (4×10⁴ cells/well, 96-plate well format, quadruplicates) were co-infected with rSARS-CoV-2 Venus (MOI 0.1) and rSARS-CoV-2 mCherry SA (MOI 0.01). After 1 h infection, p.i. media containing 3-fold serial dilutions (starting concentration 500 ng) of the indicated hMAbs was added to the cells. At 48 hpi, cells were fixed with 10% neutral buffered formalin and levels of fluorescence were quantified using a fluorescent plate reader and analyzed using Gen5 data analysis software (BioTek). NT₅₀ values of each of the hMAbs was determined using GraphPad Prism. Dashed lines indicate 50% viral neutralization. FIGS. 16A, 16B, 16C, 16D, 16E, 16F, 16G, 16H, and 16I are results for the CB6, REGN10933, REGN10987, 1206D12, 1212D5, hMAb 1215D1, 1206G12, 1212F2, and 1207B4, respectively. Data are means and SD from quadruplicate wells. Representative images are shown. Scale bars=300 μm.

### Example 13

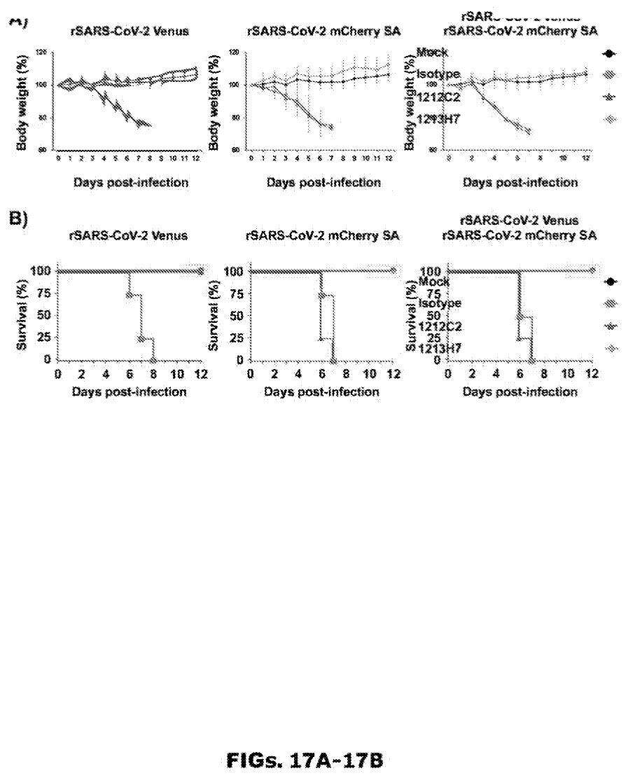

An in vivo bifluorescent-based assay to identify SARS-CoV-2 broadly Nabs. The novel bifluorescent-based assay to identify NAbs against different SARS-CoV-2 strains was adapted to assess the neutralizing activity of hMAbs in vivo. The 1212C2 and 1213H7 hMAbs were used to neutralize rSARS-CoV-2 Venus and rSARS-CoV-2 mCherry SA, alone or in combination, in the K18 hACE2 transgenic mouse model of SARS-CoV-2 infection (FIGS. 17A and 17B). Mice were treated (i.p.) with 25 mg/kg of 1212C2, 1213H7, or an IgG isotype control 24 h prior to challenge with 104 PFU of rSARS-CoV-2 Venus, rSARS-CoV-2 mCherry SA, or both rSARS-CoV-2 Venus and rSARS-CoV-2 mCherry SA together, and body weight and survival evaluated for 12 days post-infection. As expected, IgG isotype control-treated mice infected with rSARS-CoV-2 Venus, rSARS-CoV-2 mCherry SA, or both, rSARS-CoV-2 Venus and rSARS-CoV-2 mCherry SA together, exhibited weight loss starting on day 4 p.i. (FIG. 17A) and they succumbed to viral infection between days 6 to 8 p.i. (FIG. 17B). However, all mice treated with 1212C2 or 1213H7 survived challenge with rSARS-CoV-2 Venus, consistent with these two hMAbs efficiently neutralizing SARS-CoV-2 WA-1 in vitro (FIGS. 13A-13F and 15A-15F). In contrast, only 1213H7, but not 1212C2, was able to protect mice infected with rSARS-CoV-2 mCherry SA (FIGS. 17A and 17B, respectively), consistent with the inability of 1212C2 to neutralize rSARS-CoV-2 mCherry SA in vitro (FIGS. 15A-15F). As predicted, when mice were co-infected with both rSARS-CoV-2 Venus and rSARS-CoV-2 mCherry SA, only mice treated with 1213H7 retained their initial body weight and did not succumb to infection (FIGS. 17A and 17B, right panels), similar to results obtained using individual infections.

FIGS. 17A and 17B are sets of graphical representations of body weight (17A) and survival (17B) of K18 hACE2 transgenic mice treated with 1212C2 and 1213H7 against rSARS-CoV-2 Venus and rSARS-CoV-2 mCherry SA, alone or in combination. Six-to-eight-week-old female K18 hACE2 transgenic mice (n=5) were treated (i.p.) with 25 mg/kg of IgG isotype control, hMAb 1212C2, or hMAb 1213H7 and infected with 10⁴ PFU of rSARS-CoV-2 Venus (left), rSARS-CoV-2 mCherry SA (middle) or both, rSARS-CoV-2 Venus and rSARS-CoV-2 mCherry SA (right). Mice were monitored for 12 days for changes in body weight (17A) and survival (17B). Data represent the means and SD of the results determined for individual mice.

### Example 14

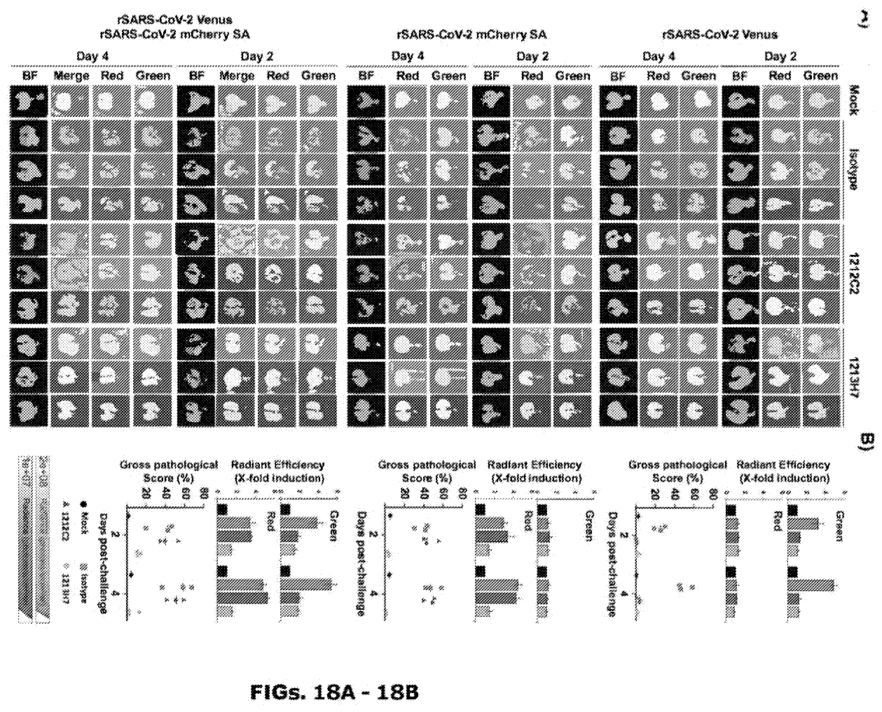

Use of FP expression to assess kinetics of SARS-CoV-2 multiplication in the lungs of infected K18 hACE2 transgenic mice. FP expression was assessed as a surrogate of virus multiplication in the lungs of infected mice and to assess the in vivo protective activity of 1212C2 and 1213H7 hMAbs using IVIS (FIGS. 18A and 18B). K18 hACE2 transgenic mice were treated (i.p., 25 mg/kg) with IgG isotype control, 1212C2, or 1213H7 hMAbs, 24 h before infection (104 PFU) with rSARS-CoV-2 Venus and/or rSARS-CoV-2 mCherry SA, alone or in combination. Mock-infected mice were included as control. At days 2 and 4 pi, Venus and mCherry expression in the lungs was evaluated using IVIS (FIG. 18A) and quantified using Aura imaging software (FIG. 18B). Excised lungs were also evaluated in a blinded manner by a certified pathologist to provide gross pathological scoring (FIG. 18A). Both Venus and mCherry expression were detected in the lungs of mice treated with the IgG isotype control and infected with rSARS-CoV-2 Venus and/or rSARS-CoV-2 mCherry SA, respectively (FIG. 18A). Fluorescent signal increased from day 2 to day 4 p.i. in the lungs of all IgG isotype control-treated infected mice (FIG. 18B). Mice treated with 1212C2 and infected with rSARS-CoV-2 Venus showed no detectable Venus signal, indicative of the ability of 1212C2 to protect against rSARS-CoV-2 Venus infection (FIG. 18A, top panel). In contrast, 1212C2-treated mice infected with rSARS-CoV-2 mCherry SA exhibited mCherry expression in the lungs (FIG. 18A, middle panel). In mice treated with 1212C2 and co-infected with both rSARS-CoV-2 Venus and rSARS-CoV-2 mCherry SA, only mCherry expression was observed, consistent with the ability of 1212C2 to neutralize rSARS-CoV-2 Venus but not rSARS-CoV-2 mCherry SA (FIG. 18A, bottom panel). Consistent with in vitro and in vivo results (FIGS. 15A-15F and 17A-17B, respectively), mice treated with 1213H7 were protected against infection with both rSARS-CoV-2 Venus and rSARS-CoV-2 mCherry SA, when administered alone or in combination, and presented no detectable fluorescence in the lungs (FIG. 18A). These data were further supported by quantification of the average radiant efficiency of fluorescence signals, which were high in the lungs of IgG isotype control-treated mice infected with rSARS-CoV-2 Venus or rSARS-CoV-2 mCherry SA, and in the lungs of 1212C2-treated mice infected with rSARS-CoV-2 mCherry SA (FIG. 18B). Importantly, gross pathological scoring correlated with levels of FP expression in the lungs of infected mice.

FIGS. 18A and 18B are sets of photographic images and graphical representations of the kinetics of fluorescent expression in the lungs of K18 hACE2 transgenic mice treated with 1212C2 or 1213H7 hMAbs and infected with rSARS-CoV-2 Venus and rSARS-CoV-2 mCherry SA. Six-to-eight-week-old female K18 hACE2 transgenic mice (n=3) were injected (i.p.) with 25 mg/kg of an IgG isotype control, hMAb 1212C2, or hMAb 1213H7 and infected with 10⁴ PFU of rSARS-CoV-2 Venus (top), rSARS-CoV-2 mCherry SA (middle), or both, rSARS-CoV-2 Venus and rSARS-CoV-2 mCherry SA (bottom). At days 2 and 4 pi, lungs were collected to determine Venus and mCherry fluorescence expression using an Ami HT imaging system (18A). Venus and mCherry radiance values were quantified based on the mean values for the regions of interest in mouse lungs (18B). Mean values were normalized to the autofluorescence in mock-infected mice at each time point and were used to calculate fold induction. Gross pathological scores in the lungs of mock-infected and rSARS-CoV-2-infected K18 hACE2 transgenic mice were calculated based on the % area of the lungs affected by infection. BF, bright field.

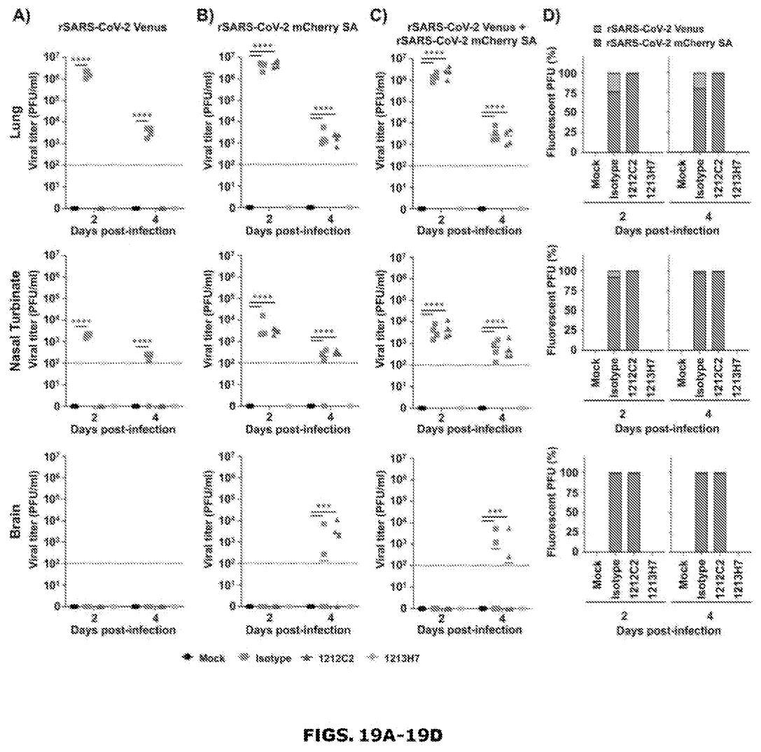

As predicted, IgG isotype control-treated K18 hACE2 transgenic mice infected with rSARS-CoV-2 Venus (FIG. 19A), rSARS-CoV-2 mCherry SA (FIG. 19B), or both rSARS-CoV-2 Venus and rSARS-CoV-2 mCherry SA (FIG. 19C) presented high viral titers. In contrast, lungs of 1212C2-treated and infected mice had undetectable levels of rSARS-CoV-2 Venus (FIG. 19A), but high titers of rSARS-CoV-2 mCherry SA (FIG. 19B) when mice were infected with each individual virus or co-infected with both viruses (FIG. 19C). In 1213H7-treated and infected mice rSARS-CoV-2 Venus (FIG. 19A) or rSARS-CoV-2 mCherry SA (FIG. 19B) were not detected including double infected mice (FIG. 19C), consistent with the ability of 1213H7 to potently neutralize both viruses in vitro and in vivo (FIGS. 15A-15F and 17A-17B, respectively). Lung homogenates from IgG isotype control-treated mice infected with both reporter viruses contained ˜25% and ˜75% of Venus and mCherry, respectively, positive plaques by day 2 pi. This finding suggested, similar to the in vitro studies (FIGS. 14A-14G), that rSARS-CoV-2 mCherry SA had a higher fitness than rSARS-CoV-2 Venus in vivo (FIG. 19D). Notably, by day 4 p.i. all viral plaques were mCherry-positive, further supporting a higher fitness of rSARS-CoV-2 mCherry SA compared to rSARS-CoV-2 Venus in vivo (FIG. 19D). Lung homogenates from 1212C2-treated mice contained rSARS-CoV-2 mCherry SA, reflecting the ability of 1212C2 to efficiently neutralize rSARS-CoV-2 Venus but not rSARS-CoV-2 mCherry SA. In contrast, no viral plaques were detected in lung homogenates from mice treated with 1213H7, as this hMAb efficiently neutralizes both viruses. Similar results were obtained in the nasal turbinate (FIGS. 19A, 19B, and 19C, middle panels) and brain (FIGS. 19A, 19B, and 19C, bottom panels) of treated and infected K18 hACE2 transgenic mice.

FIGS. 19A-19D are sets of graphical representations of viral titers in the lungs, nasal turbinate and brain, respectively, of K18 hACE2 transgenic mice treated with 1212C2 or 1213H7 hMAbs and infected with rSARS-CoV-2 Venus and rSARS-CoV-2 mCherry SA. Six-to-eight-week-old female K18 hACE2 transgenic mice (n=3) injected (i.p.) with 25 mg/kg of an IgG isotype control, hMAb 1212C2, or hMAb 1213H7 and infected with 10⁴ PFU of rSARS-CoV-2 Venus (19A), rSARS-CoV-2 mCherry SA (19B) or both, rSARS-CoV-2 Venus and rSARS-CoV-2 mCherry SA (19C). Viral titers in the lungs (top), nasal turbinate (middle) and brain (bottom) at days 2 and 4 p.i. were determined by plaque assay in Vero E6 cells. Bars indicates the mean and SD of lung virus titers. Dotted lines indicate the limit of detection. FIG. 19D is a graphical representation of the quantification of rSARS-CoV-2 Venus and rSARS-CoV-2 mCherry SA in the lungs (top), nasal turbinate (middle) and brain (bottom) from mice infected with both viruses at days 2 and 4 pi.

### Example 15

Generation of BACs with deletions of individual accessory ORF proteins. The SARS-CoV-2 genome, which was divided into 5 fragments and chemically synthesized, was assembled into a single bacterial artificial chromosome (BAC) that led to efficient virus rescue after transfection into Vero E6 cells using Lipofectamine 2000. Fragment 1 included the SARS-CoV-2 ORF accessory proteins. Using standard gene-engineering approaches, individual ORF3a, ORF6, ORF7a, ORF7b, or ORF8 were systematically deleted from fragment 1 using PCR and primer pairs containing BsaI type IIS restriction endonuclease sites. After being confirmed by Sanger sequencing (data not shown), fragment 1 containing the individual deletions of the ORF3a, ORF6, ORF7a, F1 ORF7b, or ORF8 accessory protein were reassembled into the BAC (FIG. 20).

FIG. 20 is a set of diagrammatic representations of the various genome organizations of the WT and ΔORF rSARS-CoV-2s. The SARS-CoV-2 genome includes 29.8 kb of nucleotides, among which 21.5 kb encodes the ORF1a and ORF1b replicase. The rest of the 8.3-kb viral genome encodes the structural spike (S), envelope (E), matrix (M), and nucleocapsid (N) proteins and the accessory ORF3a, 26, 27a, 27b, 28, and 210 proteins. Individual deletions of the ORF accessory proteins were introduced into the BAC for rescue of rSARS-CoV-2. Schematic representations are not drawn to scale.

### Example 16

Rescue of ΔORF rSARS-CoV-2s. BACs with individual deletions of an accessory ORF were transfected into Vero E6 cells for the recovery of ΔORF rSARS-CoV-2s. At 72 h post-transfection, tissue culture supernatants (passage 0 [P0]) were collected to inoculate fresh Vero E6 cells (P1). Supernatants were then collected from P1 at 72 h p.i., and viral titers, defined as numbers of PFU per milliliter, were determined. To verify the rescue of each ΔORF rSARS-CoV-2, indirect immunofluorescence was performed using F2 antibodies directed at the nucleocapsid (N) and spike (S) proteins (FIG. 21A). The individual deletion of each ORF from rSARS-CoV-2 was verified using reverse transcription-PCR (RT-PCR) procedures to amplify the viral N gene (control) and the regions which cover the corresponding individual ORF deletions (FIG. 21B). All the ΔORF rSARS-CoV-2s and rSARS-CoV-2/WT produced an RT-PCR product of approximately 1.2 kb corresponding to the N gene, whereas amplified regions that cover the corresponding ORF deletions were smaller in the ΔORF rSARS-CoV-2 constructs than in rSARS-CoV-2/WT (FIG. 2B), demonstrating the deletion of the individual ORFs from the viral genomes. Individual deletions of the viral proteins from the ΔORF rSARS-CoV-2s were further confirmed by Sanger sequencing of PCR products (FIG. 21C) and deep sequencing analysis of the entire viral genome (FIGS. 22A-22E). These data demonstrate that each ΔORF rSARS-CoV-2 construct contained a deletion of its individual ORF accessory protein.

FIG. 21A is a set of photographic images obtained from immunofluorescence assay using WT or the ORFΔ3a, ORFΔ6, ORFΔ7a, ORFΔ7b, or ORFΔ8 rSARS-CoV-2. Vero E6 cells (24-well plate format, 10⁵ cells/well, triplicates) were mock infected or infected (MOI of 3) with the WT or the ORFΔ3a, ORFΔ6, ORFΔ7a, ORFΔ7b, or ORFΔ8 rSARS-CoV-2. At 24 h p.i., cells were fixed and immunostained with a cross-reactive polyclonal antibody against SARS-CoV N protein and a cross-reactive monoclonal antibody against SARS-CoV S protein (3B4). Rhodamine red goat anti-rabbit and FITC rabbit anti-mouse secondary antibodies were used, and nuclei were visualized with DAPI. Scale bars, 100 mm. Vero E6 cells (6-well plate format, 10⁶ cells/well) were mock infected or infected (MOI of 0.1) with the WT or a ΔORF rSARS-CoV-2, and total RNA was extracted at 24 h p.i. Regions in the viral genome corresponding with the deletion were amplified, and the N gene was amplified as an internal control. FIG. 21B is a set of photographic images of the agarose gel separation of these amplified products. MW, molecular weight. FIG. 21C is a set of images from the sequencing of the RT-PCR products from FIG. 21B, which were gel purified and subjected to Sanger sequencing. The consensus sequences in the genomes of both the WT and a ΔORF rSARS-CoV-2 downstream of the deleted gene are indicated in blue, the intergenic regions between viral genes are shown in yellow, the genes deleted from the ΔORF rSARS-CoV-2s are indicated in green, and the viral genes upstream of the deleted ORFs in the rSARS-CoV-2s are shown in red. FIGS. 22A-22E are graphical representations from deep sequencing analysis of ORF-deficient rSARS-CoV-2s. The ORF-deficient rSARS-CoV-2 nonreference allele frequency was calculated by comparing short reads to the sequence of the respective reference WT SARS-CoV-2 USA-WA1/2020 strain genome. Silent mutations at positions 21895 and 26843 (according to the genome positions of the USA-WA1/2020 strain) were fixed in all ORF-deficient rSARS-CoV-2 genomes.

### Example 17

Characterization of the ΔORF rSARS-CoV-2 constructs in vitro. Each ΔORF rSARS-CoV-2 construct was characterized in vitro (FIGS. 23A-23E). Previous studies have shown that manipulation of the viral genome can affect the phenotypes of viral plaques; therefore, as an initial step, the impact of deletion of individual accessory ORFs on the rSARS-CoV-2 plaque phenotype was investigated. Interestingly, an effect was noticed on the plaque phenotypes of all ΔORF rSARS-CoV-2 constructs compared to that on rSARS-CoV-2/WT at all times p.i. studied (24, 48, 72, and 96 h) (FIG. 23A). These results were further confirmed by measuring the diameters of the plaques of the WT and ORF-deficient rSARS-CoV-2s on Vero E6 cell monolayers (FIG. 23B). Except for ORFΔ7b rSARS-CoV-2, all the other ORF-deficient rSARS-CoV-2s had smaller plaque phenotypes than WT rSARS-CoV-2 at any of the studied time points (FIGS. 23A and 23B). Next, the growth kinetics of the ΔORF rSARS-CoV-2s was compared to that of rSARS-CoV-2/WT in Vero E6 cells. To this end, viral titers in the tissue culture supernatants from Vero E6 cells infected (multiplicity of infection [MOI], 0.01) with the WT or ΔORF rSARS-CoV-2 collected at 12, 24, 48, 72, and 96 h p.i. were determined by plaque assay (FIG. 23B). No statistically significant differences between the WT and ORFΔ3a, ORFΔ6, and ORFΔ7b rSARS-CoV-2s were observed at any times p.i., except for the replication of ORFΔ7a and ORFΔ8 rSARS-CoV-2s, which was significantly different (1 log 10) than that of rSARS-CoV-2/WT at 12 and 24 h p.i. (FIG. 23B). Peak viral titers for all ΔORF rSARS-CoV-2s and rSARS-CoV-2/WT were observed between 24 and 48 h p.i., with viral titers decreasing (1 log 10) at later times p.i., consistent with previous studies with SARS-CoV-2 natural isolates. To further assess the effect of ORF deletions on viral fitness and replication, growth kinetics was determined in human HEK293T and Δ549 cells stably expressing the hACE2 receptor (hACE2-HEK293T and hACE2-Δ549 cells, respectively), which have been shown to be permissive to SARS-CoV-2 infection. In hACE2-HEK293T (FIG. 23D) and hACE2-Δ549 (FIG. 23E) cells, the WT and ORF-deficient rSARS-CoV-2 infections peaked at 48 h p.i. and began to decrease at the latter time points, a trend similar to that observed in Vero E6 cells (FIG. 23C). However, in contrast to the WT and other ORF-deficient rSARS-CoV-2s, both the ORFΔ7a and ORFΔ8 rSARS-CoV-2s replicated significantly less after 12 h p.i. Moreover, a decrease was observed in ORFΔ6 rSARS-CoV-2 titers after the peak at 48 h p.i. All together, these results suggest that despite slight differences in plaque phenotype, ΔORF rSARS-CoV-2s have replication kinetics similar to that of rSARS-CoV-2/WT in Vero E6 cells, while the ORFΔ7a, ORFΔ8, and ORFΔ6 rSARS-CoV-2s have altered replication capabilities in human cell lines, especially in hACE2-Δ549 cells, which are competent in innate immune responses.

FIGS. 23A-23E are photographic images and graphical representations directed to the in vitro characterization of the WT and ΔORF rSARS-CoV-2s. FIG. 23A is a set of photographic images of the plaque phenotype from Vero E6 cells (6-well plate format, 10⁶ cells/well) infected with the WT, ORFΔ3a, ORFΔ6, ORFΔ7a, ORFΔ7b, or ORFΔ8 rSARS-CoV-2 and overlaid with medium containing agar. Plates were incubated at 37° C., and monolayers were immunostained with an anti-N protein SARS-CoV cross-reactive monoclonal antibody, 1C7C7, at the indicated hours p.i. FIG. 23B is a graphical representation of the viral plaque sizes using the WT and ΔORF rSARS-CoV-2s. Diameters of viral plaques were measured with a ruler in centimeters. Plaques less than 0.1 cm are indicated as not detected (ND). FIGS. 23C-23E are graphical representations of the multicycle growth kinetics in Vero E6 cells (FIG. 23C), hACE2-HEK293T cells (FIG. 23D), and hACE2-Δ549 (FIG. 23E) cells (6-well plate format, 10⁶ cells/well, triplicates) infected (MOI 0.01) with the WT or a ΔORF rSARS-CoV-2 and incubated at 37° C. At the indicated hours p.i., tissue culture supernatants from infected cells were collected, viral titers were determined by plaque assay (PFU per milliliter), and cells were immunostained using the anti-N SARS-CoV cross-reactive monoclonal antibody 1C7C7. Data are the means 6 standard deviations (SDs) of the results determined from triplicate wells. Dotted black lines indicate the limit of detection (LOD; 100 PFU/ml). *, P, 0.05, using the Student t test. ns, not significant.

### Example 18

Characterization of ΔORF rSARS-CoV-2s in vivo. Coronavirus ORF accessory proteins have been implicated as virulence factors and contribute to both pathogenicity and disease outcome. Therefore, the contribution of SARS-CoV-2 ORF3a, ORF6, ORF7a, ORF7b, and ORF8 to viral pathogenicity and disease outcome was examined in K18 hACE2 trans-genic mouse model of SARS-CoV-2 infection and COVID-19 disease. Four- to 6-week-old female mice (n=4) were mock (phosphate-buffered saline [PBS]) infected or infected (105 PFU) with rSARS-CoV-2/WT or with one of the ΔORF rSARS-CoV-2s and observed for 14 days for morbidity (body weight loss) and mortality (survival) (FIGS. 24A-24B). Similar decreases in body weight percentages were observed up to 5 days p.i., from which ORFΔ3a and ORFΔ7b rSARS-CoV-2-infected mice began to recover (FIG. 24A). Interestingly, mice infected with ORFΔ6 and ORFΔ7a rSARS-CoV-2s continued to lose body weight until 7 and 8 days p.i., respectively, and to start to recover (FIG. 24A). All mice infected with the WT or ORFΔ8 rSARS-CoV-2 succumbed to viral infection by 6 or 7 days p.i., respectively (FIG. 24B). Observations of mice infected with ORFΔ3a, ORFΔ6a, ORFΔ7a, and ORFΔ7b rSARS-CoV-2s identified survival rates of 75%, 50%, 25%, and 25%, respectively (FIG. 24B). Upon full comparison, despite the early onset of morbidity observed in mice infected with the ORFΔ3a rSARS-CoV-2, 3 out of 4 mice survived viral infection, suggesting that ORF3a may play an important role in viral pathogenesis. This early onset of morbidity and quick recovery may be the result of an active immune response to viral infection and, therefore, viral clearance.

FIGS. 24A-24B are graphical representations of body weight (24A) and survival (25B) of K18 hACE2 transgenic mice infected with the WT or a ΔORF rSARS-CoV-2. Six- to 8-week-old K18 hACE2 transgenic female mice were mock (PBS) infected or infected (i.n.) with 10⁵ PFU of the WT or a ΔORF rSARS-CoV-2 (n=4/group). Body weight (24A) and survival (25B) were evaluated at the indicated days p.i. Mice that lost 25% of their initial body weight were humanely euthanized. Error bars represent the SDs of the mean for each group.

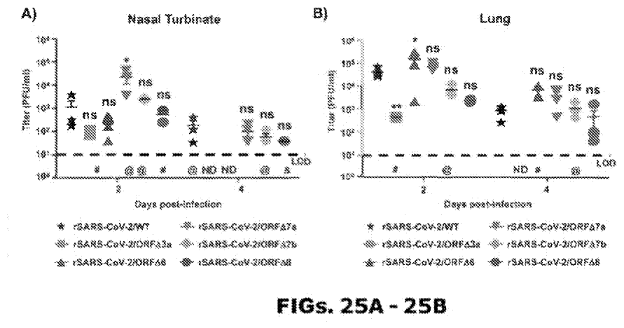

Viral replication was evaluated in nasal turbinates and lungs of K18 hACE2 transgenic mice infected with the WT or ΔORF rSARS-CoV-2 at 2 and 4 days p.i. (FIGS. 25A-25B). At 2 days p.i., the WT and ORFΔ6 rSARS-CoV-2 were detected in nasal turbinates in all mice, with rSARS-CoV-2/WT reaching titers of up to 5×103 PFU/ml, while ORFΔ6 rSARS-CoV-2 peaked at 5×102 PFU/ml. ORFΔ3a rSARS-CoV-2 (102 PFU/ml) was detected in only 50% of infected mice, while ORFΔ7b rSARS-CoV-2 replicated up to 3×103 PFU/ml in 75% of infected mice. Only 50% of mice had detectable levels (ranging between 0.5×103 and 1×103 PFU/ml) of ORFΔ8 rSARS-CoV-2 (FIG. 25A). Interestingly, out of all the ΔORF rSARS-CoV-2s tested, ORFΔ7a replicated in the nasal turbinate to levels (5×104 PFU/ml) higher than those observed with rSARS-CoV-2/WT. By 4 days p. i., ORFΔ3a and ORFΔ6 rSARS-CoV-2s were no longer detected in nasal turbinates, while 75% of mice infected with rSARS-CoV-2/WT had viral titers ranging between 5×101 and 5×102 PFU/ml. ORFΔ7a, ORFΔ7b, and ORFΔ8 rSARS-CoV-2s replicated to lower levels (5×101 PFU/ml) than rSARS-CoV-2/WT (FIG. 25A). In the lungs, the WT and ORFΔ6 and ORFΔ7a rSARS-CoV-2s were detected at levels of 10⁵ PFU/ml at 2 days p.i. (FIG. 25B). Viral titers of ORFΔ7b and ORFΔ8 rSARS-CoV-2s (;104 PFU/ml) and ORFΔ3a (;0.5×102 PFU/ml) rSARS-CoV-2 were lower than those of rSARS-CoV-2/WT (FIG. 25B). Interestingly, by 4 days p.i., ORFΔ3a rSARS-CoV-2 was no longer detected in the lungs, while WT, ORFΔ6, ORFΔ7a, ORFΔ7b, and ORFΔ8 rSARS-CoV-2s decreased only; 1 log 10 to levels observed by 2 days p.i. (FIG. 25B).

FIGS. 25A-25B are graphical representations of the titers of the WT and ΔORF rSARS-CoV-2s in nasal turbinate and lungs, respectively. Six- to 8-week-old K18 hACE2 transgenic female mice were mock (PBS) infected or infected (i.n.) with 10⁵ PFU of the WT or a ΔORF rSARS-CoV-2 (n=8/group). Mice were sacrificed at 2 (n=4/group) and 4 (n=4/group) days p.i., and viral titers in nasal turbinate and lung were determined by plaque assay (PFU per milliliter) and immunostaining using the cross-reactive SARS-CoV 1C7C7 N protein monoclonal antibody. Viral titers in the nasal turbinate (25A) and lungs (25B) are shown. Symbols represent data from individual mice and bars the geometric means of viral titers. Dotted lines indicate the LOD (10 PFU/ml). ND, not detected; @, not detected in 1 mouse; #, not detected in 2 mice: &, not detected in 3 mice. Negative results of the PBS-infected mice are not plotted.

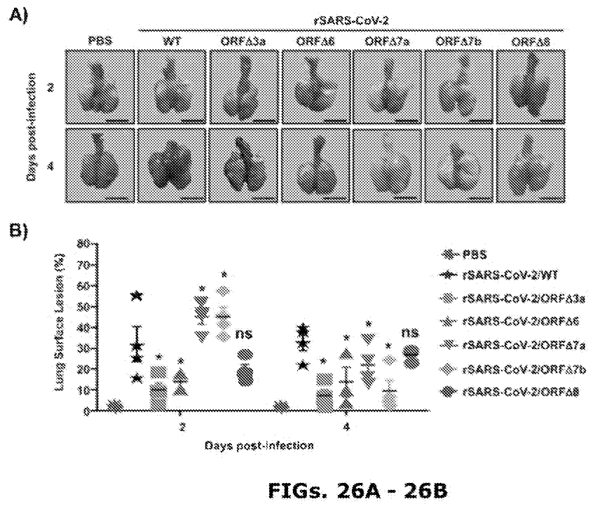

Furthermore, to evaluate the impact of viral infection in the lungs of infected animals, gross pathology analysis was performed on lungs collected at 2 and 4 days p.i. (FIG. 26A). Both the WT and ΔORF rSARS-CoV-2s induced similar pathologies at 2 days p.i., with the WT, ORFΔ7a, and ORFΔ7b rSARS-CoV-2s inducing pathological lesions in more than 50% of the lung area (FIG. 26B). Intriguingly, by 4 days p.i., only WT and ORFΔ8 rSARS-CoV-2-infected lungs maintained pathological lesions, while ORFΔ3a, ORFΔ6, ORFΔ7a, and ORFΔ7b rSARS-CoV-2-infected lungs appeared to recover from lesions observed at 2 days p.i. All together, these in vivo results provide new insight into the contribution of SARS-CoV-2 accessory ORF proteins in viral pathogenesis and disease, suggesting a major role of ORF3a and ORF6 and a smaller impact of ORF7a, ORF7b, and ORF8 on virulence and disease outcome. FIGS. 26A-26B are photographic images and a graphical representation from the gross pathology analysis of lungs from K18 hACE2 transgenic mice infected with the WT or a ΔORF rSARS-CoV-2. Lungs from 6-to-8-week-old female K18 hACE2 transgenic mice (n=8/group) mock (PBS) infected or infected (i.n.) with 105 PFU of the WT or a ΔORF rSARS-CoV-2 were harvested at 2 (n=4/group) or 4 (n=4/group) days p.i., imaged (26A), and scored for virally induced lesions (26B). The total lung surface area affected by virally induced lesions were determined. Scale bars, 3 cm.

The induction of chemokines and cytokines during infection in the lung was evaluated using an 8-plex Luminex assay (FIG. 27). Infection of K18 hACE2 transgenic mice with the SARS-CoV-2 WA-1/US strain induces local chemokine and cytokine storms in the lung. Compared to mock-infected K18 hACE2 transgenic mice, infection of mice with rSARS-CoV-2/WT induced a significant production of type I interferon (IFN-α) and type II IFN (IFN-γ) responses and chemo-attractants (e.g., CCL5/RANTES) at 2 days p.i. (FIG. 27A). However, production of tumor necrosis factor alpha (TNF-α) (TH1), interleukin 6 (IL-6), IL-10 (TH2), and IL-17 (TH17) responses were low in rSARS-CoV-2/WT-infected K18 hACE2 transgenic mice (FIG. 27A). All the ORF-deficient rSARS-CoV-2 constructs induced IFN responses at different levels, and the highest production of IFN- and CCL5/RANTES was observed in mice infected with ΔORF6 rSARS-CoV-2. At 2 days p.i., no significant TH1 and TH2 responses were observed with any of the ORF-deficient rSARS-CoV-2s, except for ΔORF3a SARS-CoV-2, which induced significant levels of IL-10 (TH2). Conversely, elevated TH17 responses were observed for both ΔORF7b and ΔORF8 rSARS-CoV-2s. At 4 days p.i., all immuno-modulator levels dropped to become non-significant compared to levels in rSARS-CoV-2/WT-infected K18 hACE2 transgenic mice, except for the levels of TNF-α induced by ΔORF7a SARS-CoV-2 infection, which remained significantly elevated. Importantly, when looking at predictors of the cytokine storm outcome, the IL-6/IL-10 ratio was lower for ΔORF3a rSARS-CoV-2. Together with ΔORF3a rSARS-CoV-2 inducing early onset of morbidity, less mortality, lower lung viral titers, and less tissue damage, these results indicate a major role for ORF3a in viral pathogenesis. Interestingly, ΔORF8 rSARS-CoV-2 behaved like rSARS-CoV-2/WT (e.g., higher morbidity/mortality and viral titers), and as such, this mutant virus also presented the highest IL-6/IL-10 ratio at 2 days p.i. (FIG. 27B). As expected, the IL-6/IL-10 ratio became normalized across all rSARS-CoV-2 constructs evaluated by 4 days p.i. (FIG. 27B).

FIG. 27A is a set of graphical representations of the cytokine and chemokine storms in the lungs of K18 hACE2 transgenic mice mock infected and infected with the WT or a ΔORF rSARS-CoV-2 were determined using an 8-plex panel mouse ProcartaPlex assay. FIG. 27B is a schematic representation of the IL-6/IL-10 ratio as a marker of the local cytokine storm induced by rSARS-CoV-2. A two-way ANOVA of K18 hACE2 transgenic mice mock infected or infected with the WT or an ORF-deficient rSARS-CoV-2 was carried out with 4 mice per time point, except for the mock-infected group (n=2).

FIG. 28A is a schematic representation of the rSARS-CoV-2 genomes. The viral spike (S), envelope (E), matrix (M), and nucleocapsid (N) structural proteins; and the accessory 1a, 1b 3a, 6, 7a, 7b, 8, and 10 open reading frame (ORF) proteins are indicated. Double ORF deletions were introduced into the BAC for rescue of rSARS-CoV-2. Schematic representation not drawn to scale. FIG. 28B is a set of photographic images of RT-PCR products of regions in the viral genome corresponding to the deletions in the viral genome as analyzed on a 0.8% agarose gel. The viral N gene was amplified as internal control. Vero E6 cells (6-well plate format, 10⁶ cells/well) were mock-infected or infected (MOI 0.1) with WT or the indicated double ΔORF rSARS-CoV-2 and total RNA was extracted at 24 h p.i and subject to RT-PCR amplification and products were analyzed on a 0.8% agarose gel. FIG. 28C is a set of representations from deep sequencing analysis of the double ORF deficient rSARS-CoV-2. Double ORF deficient rSARS-CoV-2 were deep sequenced and the non-reference allele frequency was calculated by comparing short reads to the respective WT rSARS-CoV-2 reference genome. Non-reference alleles present in less than 1% of reads are not shown (dotted line). Amino acid changes respective to rSARS-CoV-2 WT are shown.

To verify the rescue, RT-PCR was used to amplify ORF3a, ORF6, ORF7a and ORF7b from the double deletion rSARS-CoV-2-infected Vero E6 cellular total RNA (FIG. 28B). ORF3a could be readily amplified from wild-type rSARS-CoV-2 infected Vero E6 cellular total RNA, but not from all the double deletion rSARS-CoV-2 (FIG. 28B, top panel). Similarly, ORF6, ORF7a and ORF7b were not amplified from the corresponding double deletion rSARS-CoV-2, but from other double deletion rSARS-CoV-2 (FIG. 28B, middle panels). As an internal control N was amplified and detected in all samples (FIG. 28B, bottom panel). To further confirm the sequences of the double deletion rSARS-CoV-2, these viruses were also subjected to next generation sequencing (FIG. 28C). In total, four non-reference alleles were found in these viruses with the percentage greater than 10%. Among them, two non-reference alleles (Spike N74K and ORF8 S21G) were found in rSARS-CoV-2 ΔORF3a/ΔORF6 with the percentage of 58.52% and 57.69% (FIG. 28C, top panel), one non-reference allele (ORF8 Δ51G) was discovered in rSARS-CoV-2 ΔORF3a/ΔORF7a with the percentage of 12.04% (FIG. 28C, middle panel), and the last non-reference allele (Envelop L37H) was present in rSARS-CoV-2 ΔORF3a/ΔORF7b with the percentage of 14.94% (FIG. 28C, bottom panel).

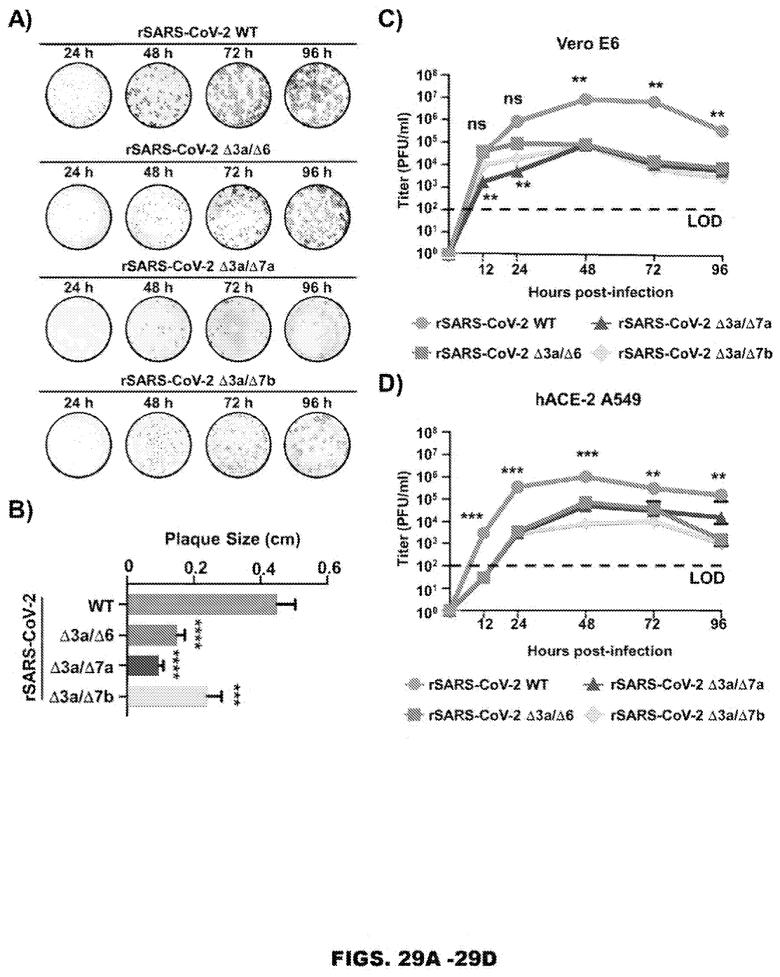

Each of the double ΔORF rSARS-CoV-2 constructs were characterized in vitro. Generally, attenuation of viruses hinders their ability to disseminate or subvert host cellular pathways. It has been observed that attenuation of respiratory viruses such as respiratory syncytial virus (RSV) and IAV leads to significant changes in the morphology of viral plaques and fitness. Based on this premise and the previous observations with single ΔORF rSARS-CoV-2, plaque assays were performed to determine if the double ΔORF rSARS-CoV-2 mutants exhibited a change in plaque morphology and fitness (FIGS. 29A-29D).

FIG. 29A is a set of photographic images of the plaque assay for in vitro characterization of double ΔORF rSARS-CoV-2. Vero E6 cells (6-well plate format, 10⁶ cells/well) were infected with WT, ORFΔ3a/Δ6, ORFΔ3a/Δ7a, or ORFΔ3a/Δ7b rSARS-CoV-2 and overlaid with media containing agar. Plates were incubated at 37° C. and monolayers were immunostained with an anti-N monoclonal antibody (1C7C7) at the indicated h.p.i. FIG. 29B is a set of graphical representations of viral plaque sizes of double ΔORF rSARS-CoV-2 constructs. Diameter of plaques size from panel A were measured with a standard ruler in centimeters (cm). FIGS. 29C-29D are graphical representations of the multicycle growth kinetics of the double ΔORF rSARS-CoV-2 constructs in Vero E6 (C) and hACE-2 Δ549 (D) cells. The cells (6-well plate format, 10⁶ cells/well, triplicates) were infected (MOI 0.01) with WT and double ΔORF rSARS-CoV-2 and incubated 37° C. At the indicated h p.i. tissue culture supernatants from infected cells were collected and viral titers were determined by plaque assay (PFU/ml) and immunostaining using an anti-N monoclonal antibody (1C7C7). Data represent the means+/−standard deviations (SDs) of the results determined in triplicate wells. Dotted black lines indicate the limit of detection (LOD, 100 PFU/ml). P<0.05: using the Student T test.

To this end, VeroE6 confluent monolayers were infected with 10-fold serial dilutions of each double ΔORF rSARS-CoV-2. Monolayers were fixed at 24, 48, 72, and 96 hours post infection (h p.i.) and immunostained using the SARS-CoV-2 anti-N monoclonal antibody 1C7C7 (FIG. 29A). In contrast to the rSARS-CoV-2 WT, all mutants exhibited smaller plaque diameter, with rSARS-CoV-2 Δ3a/Δ7b forming plaque sizes greater than 0.2 cm in diameter, about half the size of the rSARS-CoV-2 WT (FIG. 29B). To determine if the double ORF rSARS-CoV-2 mutants where attenuated in cell culture, growth kinetic studies were carried out in VeroE6 and Δ549 cells expressing the hACE-2 receptor (hACE-2 Δ549) (FIGS. 29C and 29D, respectively). Confluent monolayers of VeroE6 or hACE-2 Δ549 cells were infected with rSARS-CoV-2 WT, ΔORF3a/Δ6, ΔORF3a/Δ7a, or ΔORF3a/Δ7b, at a multiplicity of infection (MOI) of 0.01, and tissue culture supernatants collected at 12, 24, 48, 72, and 96 h p.i. All mutants exhibited attenuated replication specially after 12 h p.i. in VeroE6 cells, and at all time points in the hACE-2 Δ549 cell line. These results indicate that the double ΔORF rSARS-CoV-2 mutants exhibit properties of attenuation.

Coronavirus ORFs have been implicated in subversion of host cellular pathways in order to facilitate viral replication and dissemination. Furthermore, infection with a rSARS-CoV-2 deficient in ORF3a results in a survival rate of 75%, therefore, all three double ΔORF rSARS-CoV-2 mutants were designed to result in 100% survival and not induce any signs of disease in the K18 hACE-2 mouse model of SARS-CoV-2 infection. Four-to-six-week-old female mice (n=4) were mock (PBS)-infected or infected with 105 PFU of WT or double ΔORF rSARS-CoV-2 and observed for 21 days for morbidity and survival (FIGS. 30A-30D).

FIGS. 30A-30B are graphical representations of the body weight (A) and survival (B) of K18 hACE2 transgenic mice infected with double ΔORF rSARS-CoV-2. K18 hACE2 transgenic female 6-8-week-old mice (N=13/group) were mock (PBS)-infected or infected (i.n.) with 10⁵ PFU of WT (pink circles) or ORFΔ3a/Δ6 (red squares), ORFΔ3a/Δ7a (blue triangles), or ORFΔ3a/Δ7b (green diamonds) double ΔORF rSARS-CoV-2. Body weight (A) and survival (B) (n=5/group) were evaluated at the indicated d p.i. Error bars represent SDs of the mean for each group. Mice were sacrificed at 2 (n=4/group) and 4 (n=4/group) d p.i. FIGS. 30C-30D are graphical representations of the viral titers in nasal turbinate (C) and lungs (D) of K18 hACE2 transgenic mice infected with double ΔORF rSARS-CoV-2 as determined by plaque assay (PFU/ml) and immunostaining using the cross-reactive SARS-CoV 1C7C7 N protein monoclonal antibody. LOD, limit of detection (100 PFU/ml). One-way ANOVA with multiple comparisons.

In contrast to the WT infected mice, mice infected with double ΔORF rSARS-CoV-2 maintained a steady weight average, with a maximum loss of 5% in the rSARS-CoV-2 Δ3a/Δ7a and Δ3a/Δ7b infected groups observed at 10 days post infection (d p.i.). Additionally, the rSARS-CoV-2 Δ3a/Δ6 infected group-maintained weight throughout the 21 days of observation (FIG. 30A). Despite their minimal weight loss, one mouse (25%) succumbed to rSARS-CoV-2 Δ3a/Δ7a infection at day 11 p.i., while the rSARS-CoV-2 Δ3a/Δ6 and Δ3a/Δ7b infected groups had a 100% survival rate (FIG. 30B). All mice infected with rSARS-CoV-2 WT gradually lost weight and succumbed to infection by 7 d p.i., as expected (FIGS. 30A and 30B, respectively).

Viral replication in the nasal turbinate and lungs of K18 hACE-2 transgenic mice infected with the WT or double ΔORF rSARS-CoV-2 was evaluated at two and four d p.i. (FIGS. 30C-30D). By two d p.i. WT and Δ3a/Δ7a rSARS-CoV-2 were detected in three of four mice, reaching viral titers ranging from 5×10²-5×10³ PFU/mL, while Δ3a/Δ7b was detected and in all four mice with viral titers similar to the WT (1×10³ PFU/mL) (FIG. 30C). Surprisingly, rSARS-CoV-2 Δ3a/Δ6 was not detected at either two or four d p.i. in the nasal turbinates (FIG. 30D). In the lungs, both WT and Δ3a/Δ7b rSARS-CoV-2 were detected in all four mice with comparable viral titers, -5×10⁵ PFU/mL at 2 d p.i., while Δ3a/Δ6 and Δ3a/Δ7a rSARS-CoV-2 reached viral titers of up to 5×10⁴ PFU/mL in all four mice at two d p.i. At four d p.i. all four mice had viral titers of -5×10⁴ PFU of the WT rSARS-CoV-2 and only three mice had detectable Δ3a/Δ7b viral titers of up to -5×10³ PFU/mL. Moreover, both Δ3a/Δ6 and Δ3a/Δ7a rSARS-CoV-2 were no longer detected in the lungs at four d p.i. (FIG. 30D).

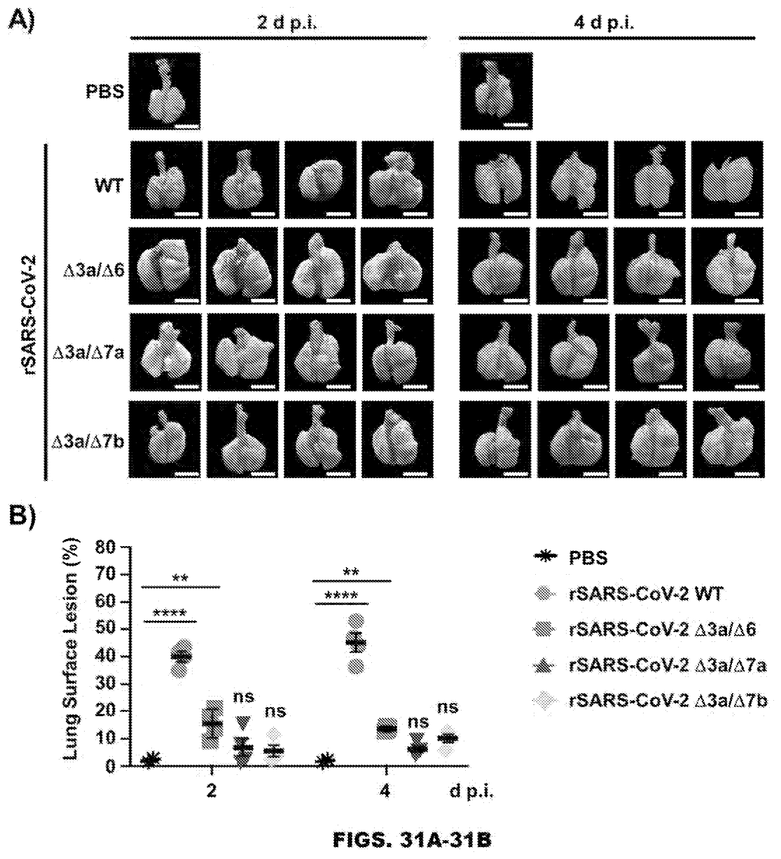

Furthermore, to determine the impact of viral replication in the lungs of infected animals, gross pathology analysis was performed on lungs collected at two and four d p.i. (FIGS. 31A-31B). FIGS. 31A-31B are photographic images and a graphical representation from the gross pathology analysis of lungs from K18 hACE2 transgenic mice infected with double ΔORF rSARS-CoV-2. Lungs from 6-to-8-week-old female K18 hACE2 transgenic mice mock (PBS)-infected or infected (i.n.) with 10⁵ PFU of WT or ORFΔ3a/Δ6, ORFΔ3a/Δ7a, or ORFΔ3a/Δ7b double ΔORF rSARS-CoV-2, were collected at 2 (n=4/group; left) or 4 (n=4/group, right) d p.i., imaged (FIG. 31A) and scored for viral induced lung lesions (FIG. 31B). Calculation of total lung surface area affected by viral induced lesions. Scale bars, 3 cm. Only the WT rSARS-CoV-2 induced pathological lesions in 40-50% of total lung area, while the double ΔORF mutants induced lesions ranging from 5-20% of lung area, Δ3a/Δ7b≤Δ3a/Δ7a<Δ3a/Δ6. By four d p.i. WT rSARS-CoV-2 induced lesions ranged from 40-60% of total lung area while lesions induced by the double ΔORF rSARS-CoV-2 mutants decreased to mock levels or remained within a similar percentage as observed at two d p.i. (FIG. 31B).

### Example 19

The rSARS-CoV-2 Δ3a/Δ7a and Δ3a/Δ7b constructs induce production of antibodies against SARS-CoV-2 WT and emerging variants. Survival of the groups infected with the double ΔORF rSARS-CoV-2 mutants was suggestive of viral attenuation in vivo. Therefore, at 14 d.p.i. and 21 d p.i., sera was collected from mice infected with rSARS-CoV-2 Δ3a/Δ6, Δ3a/Δ7a, or Δ3a/Δ7b and evaluated for total immunoglobulin (Ig) production and neutralization capacity. Using cell extracts from VeroE6 cells infected with SARS-CoV-2 WA-1/2020, total Ig ELISA was performed with two-fold serial dilutions, starting at 1:100 of serum. As a positive control, the anti-SARS-CoV-2 N antibody, 1C7C7, was used.

FIGS. 32A-32G are graphical representations of the humoral induced by Double ΔORF rSARS-CoV-2 responses against SARS-CoV-2 variants. K18 hACE2 transgenic female 6-8-week-old mice were mock (PBS)-infected or infected (i.n.) with 10⁵ PFU of ORFΔ3a/Δ6, ORFΔ3a/Δ7a, or ORFΔ3a/Δ7b double ΔORF rSARS-CoV-2 (n=4/group). At 14 (A) and 21 (B) d p.i., mice were bled and sera were collected and evaluated individually for the presence of total antibodies by ELISA (A-B). As a positive control, the SARS-CoV-2 anti-N monoclonal antibody (1C7C7) was used. Serum collected at 21 d p.i. were evaluated for their capacity to neutralize SARS-CoV-2 WA-1/2020 (C), a (D), 3 (E), and 8 (F) VoC NT₅₀ values for each assay are shown in G.

All samples, 14 d p.i. (FIG. 32A), 21 d p.i. (FIG. 32A) and the positive control, were processed on a single plate. Sera collected from mice infected with rSARS-CoV-2 Δ3a/Δ7b at 14 and 21 d p.i. (FIG. 32A and FIG. 32B, green diamonds, respectively) was able to potently detect SARS-CoV-2 WA-1/2020, while serum from mice infected with rSARS-CoV-2 Δ3a/Δ7a was only able to detect SARS-CoV-2 WA-1/2020 at 21 d p.i. (FIG. 32A and FIG. 32B, blue triangles, respectively). Interestingly, serum collected from rSARS-CoV-2 Δ3a/Δ6 (FIG. 32A and FIG. 32B, red squares, respectively) failed to detect SARS-CoV-2 WA-1/2020 at either 14 or 21 d p.i.

Serum collected at day 21 was evaluated for neutralizing antibodies (NAbs) against the SARS-CoV-2 USA-WA1/2020 isolate (FIG. 32C), SARS-CoV-2 α (FIG. 32D), SARS-CoV-2 β (FIG. 32E), and SARS-CoV-2 ε (FIG. 32F). Sera collected from mice infected with rSARS-CoV-2 ΔORF3a/Δ7a ΔORF3a/Δ7b was able to neutralize all SARS-CoV-2 strains with NT₅₀ (50% virus neutralization) titers ranging from 1:300 to 1:500. Surprisingly, serum from the rSARS-CoV-2 ΔORF3a/Δ6 group did not show any neutralizing capacity against any of the strains (FIG. 32G). Altogether, the data suggest that rSARS-CoV-2 ΔORF3a/Δ7b should be further studied for a potential LAV against SARS-CoV-2. Despite presenting with 25% mortality, the surviving mice from the rSARS-CoV-2 Δ3a/Δ7a group were able to seroconvert and produce neutralizing antibodies against SARS-CoV-2. Surprisingly, the group infected with rSARS-CoV-2 Δ3a/Δ6 were not able to generate any antibody production.

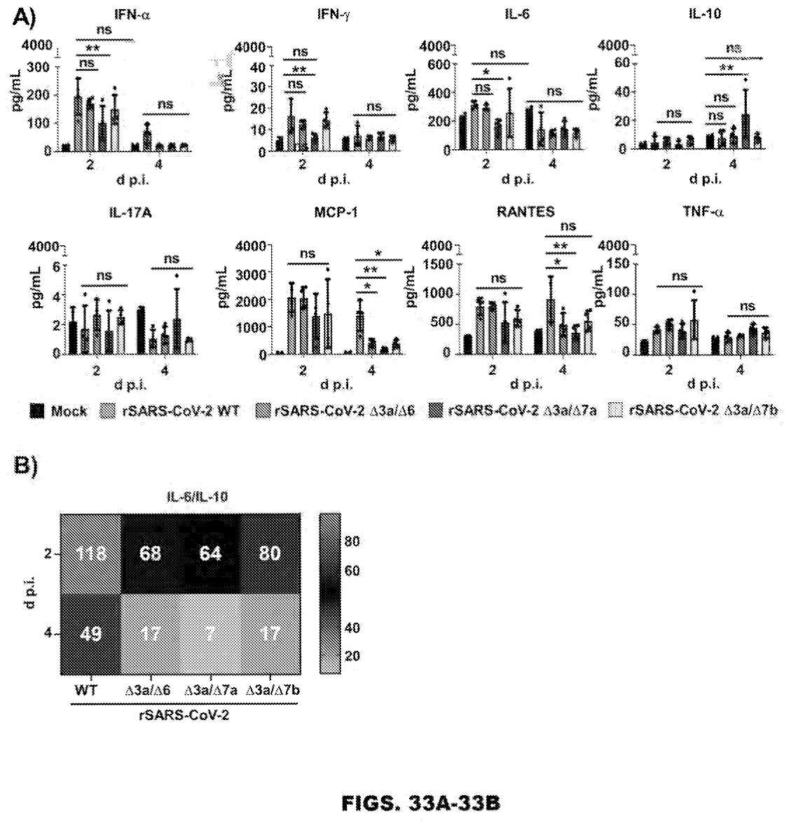

Analyses of Th1/Th2/Th17 immune responses and chemo-attractants present in the lungs of K18 hACE2 transgenic mice infected with the different double ΔORF rSARS-CoV-2 mutants were performed (FIGS. 33A-33B). FIGS. 33A-33B are graphical representations of the cytokine and chemokine levels induced by double ΔORF rSARS-CoV-2 infection. Cytokine and chemokine levels (pg/ml) were measured in lung homogenates of K18 hACE2 transgenic mice infected with WT or double ΔORF rSARS-CoV-2 mutants at (A) 2 dpi and (B) 4 dpi. IL-6/IL-10 ratio is shown for each of the time points as a marker of the local cytokine storm. Data is represented as M±SD with n=4 mice per group and time point; one-way ANOVA with multiple comparisons: *p<0.05, **p<0.005, ***p<0.0005, ****p<0.00005.

At day two post-infection, there were almost no differences among all virus strains studied, except for the rSARS-CoV-2 Δ3a/7Aa mutant, which showed significantly lower levels of induced interferon responses (e.g. IFN-α and IFN-γ) (FIG. 33A). However, by day four post-infection, all mutants showed a decrease in inducing IFN-α when compared to the rSARS-CoV-2 WT strain. This decrease was not observed for IFN-γ. All mutants also significantly induced lower levels of chemo-attractants by day four post-infection. Interestingly, rSARS-CoV-2 Δ3a/Δ7a mutant drove the induction of higher levels of TNF, IL-17A and IL-10, and the lowest IL-6/IL-10 ratio (FIG. 33B). This is important, as higher IL-6/IL-10 ratio values are correlated to worse outcome linked to the cytokine storm development.

### Example 20

Vaccination with rSARS-CoV-2 Δ3a/Δ6 or rSARS-CoV-2 Δ3a/Δ7b confer protection from lethal challenge with rSARS-CoV-2 Nluc-2A. To demonstrate the feasibility of rSARS-CoV-2 Δ3a/Δ6 or rSARS-CoV-2 Δ3a/Δ7b as LAVs against SARS-CoV-2, challenge and protection experiments were performed. FIGS. 34A-34D are graphical representations of the in vivo protection efficacy of double ΔORF rSARS-CoV-2. K18 hACE2 transgenic female 6-8-week-old mice were mock (PBS)-infected (pink circles) or infected (i.n.) with 10⁵ PFU of Δ3a/Δ6 or rSARS-CoV-2 Δ3a/Δ7b rSARS-CoV-2 (n=5/group). Body weight (A) and survival (B) were evaluated at the indicated d p.i. (n=5/group). Error bars represent SDs of the mean for each group. At 21 d p.i. mice where challenged with 105 PFU of rSARS-CoV-2 Nluc-2A and body weight (C) and survival (D) were evaluated for 10 days. Error bars represent SDs of the mean for each group.

Four-to-six-week-old female mice (n=4) were mock (PBS)-vaccinated or vaccinated with 105 PFU of rSARS-CoV-2 Δ3a/Δ6 or rSARS-CoV-2 Δ3a/Δ7b and observed daily for 21 days for morbidity and survival (FIGS. 34A and 34B, respectively). At 21 d p.i., mice were challenged with 10⁵ PFU of rSARS-CoV-2 Nluc-2A and monitored daily for morbidity and survival (FIGS. 34C and 34D, respectively). As expected, the PBS vaccinated mice challenged with rSARS-CoV-2 Nluc-2A began to show signs of disease by 3 d p.i. (FIG. 34C) and succumbed to infection by 7 d p.i. (FIG. 34D), additionally, the mice vaccinated with rSARS-CoV-2 Δ3a/Δ7b had no clinical signs of disease and had a 100% survival rate (FIGS. 34A and 34B, respectively, green diamonds). Despite not showing any total Ig production against SARS-CoV-2 WA-1/2020 natural isolate (FIGS. 32A and 32B, red squares) or neutralizing antibody production against SARS-CoV-2 WA-1/2020 natural isolate or any of the emerging variants (FIGS. 32C-32F, red squares), mice vaccinated with rSARS-CoV-2 Δ3a/Δ6 demonstrated no signs of disease and had a 100% survival rate (FIGS. 34C and 34D, respectively, red squares).

### Example 21

Viral replication was evaluated by real-time in the whole mouse and determined viral titer in the nasal turbinates and lungs of the vaccinated and challenged mice. FIG. 35A is a set of photographic images of the expression of rSARS-CoV-2 Nluc in double ΔORF rSARS-CoV-2 vaccinated mice. K18 hACE2 transgenic female 6-8-week-old mice (n=8/group) were mock (PBS)-infected or infected (i.n.) with 10⁵ PFU of Δ3a/Δ6 or rSARS-CoV-2 Δ3a/Δ7b rSARS-CoV-2. At 21 d.p.i. mice where mock-challenged or challenged (i.n.) with 10⁵ PFU of rSARS-CoV-2 Nluc-2A. Mice (n=4/group) were anesthetized at 1, 2, 4 and 6 d p.i. and retro-orbitally injected with the Nluc substrate. Nluc expression was determined using an AmiHT system. FIG. 35B is a graphical representation of the expression of rSARS-CoV-2 Nluc in double ΔORF rSARS-CoV-2 vaccinated mice as analyzed by the Aura program. FIGS. 35C-35D are graphical representations of the viral titers in nasal turbinate (C) and lungs (D) of double ΔORF rSARS-CoV-2 vaccinated mice, as determined by plaque assay (PFU/ml) and immunostaining using the cross-reactive SARS-CoV 1C7C7 N protein monoclonal antibody. LOD, limit of detection (100 PFU/ml).

By utilizing the rSARS-CoV-2 Nluc-2A as the challenge virus, it was possible to monitor live virus infection in real-time using the Ami HT Imaging system at days 1, 2, 4, and 6 post challenge (FIG. 35A). Efficient detection of viral replication in the PBS vaccinated mice occurred as early as one d p.i., indicated by increasing luciferase readout up to day 6 p.i. (FIG. 35B), by which 50% of mice had succumbed to viral infection. In both vaccinated groups, rSARS-CoV-2 Δ3a/Δ6 and rSARS-CoV-2 3a/Δ7b, there was no luciferase signal and viral replication was detected at any day post challenge (FIGS. 35A and 35B). Evaluation of viral titer in the nasal turbinate (FIG. 35C) and lungs (FIG. 35D) correlated with the in vivo imaging data (FIGS. 35A and 35B), with viral titers only detected in the PBS vaccinated group (pink circles) at both 2 and 4 d p.i., while both rSARS-CoV-2 Δ3a/Δ6 and rSARS-CoV-2 3a/Δ7b vaccinated groups had no detectable viral titer at either 2 or 4 d p.i. (red squares and green triangles, respectively). Vaccination with either rSARS-CoV-2 Δ3a/Δ6 and rSARS-CoV-2 3a/Δ7b can elicit a protective response to lethal challenge with SARS-CoV-2 and can serve as a live attenuated vaccine.

### Example 22

Lethal challenge with rSARS-CoV-2 Nluc-2A of rSARS-CoV-2 Δ3a/Δ6 and rSARS-CoV-2 3a/Δ7b vaccinated mice induces a delayed and controlled immune response. FIGS. 36A-36G are graphical representations of the cytokine and chemokine levels of vaccinated mice after challenge with rSARS-CoV-2 Nluc-2A. Cytokine and chemokine levels were measured in lung homogenates of K18 hACE2 transgenic mice mock vaccinated (pink) or vaccinated with double ΔORF rSARS-CoV-2 mutants ORFΔ3a/Δ6 (red), or ORFΔ3a/Δ7b (green) mutants at 2 dpi (n=4/group) and 4 dpi (n=4/group). Mice were challenged with 105 PFU of rSARS-CoV-2 Nluc-2A. (A). IL-6/IL-10 ratio is shown for each of the time points as a marker of the local cytokine storm (B). Data represent the means+/−SDs of the results for 4 individual mice. One-way ANOVA with multiple comparisons: ns, not significance, *p<0.05, **p<0.005, ***<0.0005.

As expected, PBS vaccinated groups upon challenge with rSARS-CoV-2 Nluc-2A produced a marked interferon response indicated by a significant production of IFN Type I (IFN-α) and II (IFN-γ) (FIG. 36A, pink bars). This was also accompanied by a high production of IL-6, driving a high IL-6/IL-10 ratio (FIG. 36B), which combined with induced elevated levels of chemo-attractants maintained overtime, drives the cytokine/chemokine storm described during SARS-CoV-2 infection. However, rSARS-CoV-2 Nluc-2A challenge groups vaccinated with either rSARS-CoV-2 Δ3a/Δ6 (red bars) or rSARS-CoV-2Δ3a/Δ7b (green bars) do not produce IFN responses, nor even more importantly IL-6, but IL-10 was induced, driving a low IL-6/IL-10 ratio accompanied by reduced production of chemo-attractants (FIG. 36B). Interestingly, upon challenge both vaccinated groups also produced a protective TH17 response, marked by their elevated production of IL-17A when compared to the PBS vaccinated group. This IL-17A production also accompanied by an elevated production of TNF was observed at 4 d p.i.

### Example 23—Materials and Methods

Biosafety. All the in vitro and in vivo experiments with infectious SARS-CoV-2 were conducted under appropriate biosafety level 3 (BSL3) and animal BSL3 (ABSL3) laboratories, respectively, at the Texas Biomedical Research Institute (Texas Biomed). Experiments were approved by the Texas Biomed Institutional Biosafety Committee (IBC) and Institutional Animal Care and Use Committee (IACUC).

Cells and virus. African green monkey kidney epithelial cells (Vero E6, CRL-1586) were obtained from the American Type Culture Collection (ATCC; Bethesda, MD). These Vero E6 cells were grown and maintained in Dulbecco's modified Eagle's medium (DMEM) supplemented with 10% fetal bovine serum (FBS) and 1% PSG (100 units/ml penicillin, 100 μg/ml streptomycin, and 2 mM L-glutamine), at 37° C. with 5% C02. For certain experiments, these cells were maintained in Dulbecco's modified Eagle medium (DMEM) supplemented with 5% (vol/vol) fetal bovine serum (FBS; VWR) and 100 units/ml penicillin-streptomycin (Corning).

The SARS-CoV-2 USA-WA1/2020 natural isolate was obtained from BEI Resources (NR-52281) and amplified on Vero E6 cells. This strain was selected because it was isolated from an oropharyngeal swab from a patient with respiratory illness in January 2020 in Washington, DC. The SARS-CoV-2 USA-WA1/2020 sequence was available from GenBank (accession no. MN985325).

Sequencing. Short-read sequencing libraries were generated from the BAC and recovered SARS-CoV-2 viral RNA. For BAC sequencing, the PCR-free KAPA HyperPlus kit protocol was followed, using 500 ng of input RNA. For SARS-CoV-2 viral RNA sequencing, libraries were generated using a KAPA RNA HyperPrep kit with a 45-min adapter ligation incubation, including 6 cycles of PCR with 100 ng RNA and a 7 mM adapter concentration. Samples were sequenced on an Illumina HiSeq X machine. Raw reads were quality filtered using Trimmomatic v0.39 (32) and mapped to a SARS-CoV-2 reference genome (GenBank accession no. MN985325) with Bowtie2 v2.4.1. Genome coverage was quantified with MosDepth v0.2.6. Each sample was genotyped for low-frequency variants with LoFreq* v2.1.3.1 (35) and filtered sites with less than a 100read depth or minor allele frequencies less than 1%. SnpEff v4.3t was used to identify the impact of potential variants on the protein coding regions in the SARS-CoV-2 reference genome. For certain experiments, viral RNAs from Vero E6 cells (1.2×10⁶ cells/well, 6-well plate format) infected at multiplicity of infection (MOI) of 0.01 were extracted using TRIzol reagent (Thermo Fisher Scientific), according to the manufacturer's specifications. Libraries were generated with a KAPA RNA HyperPrep kit, 100 ng of RNA, and 7 mM of adapter. The Illumina HiSeq X was used for sequencing. Raw reads were filtered using Trimmomatic v0.39 (REF), mapped to the SARS-CoV-2 USA/WA1/2020 reference genome with Bowtie v2.4.1 (REF), and the total genomic coverage was quantified using MosDepth v0.2.6 (REF). Genotypic analysis was conducted with LoFreq* v2.1.3.1 (REF) to filter low-frequency variants, in which sites with less than a 100× read depth or a 1% minor allele frequency were eliminated. Lastly, potential variants and their influence on protein coding regions were identified with SnpEff v4.3t (REF), using SARS-CoV-2 USA/WA1/2020 reference genome.

Assembly of the full length of the SARS-CoV-2 genome. Based on genomic information of the SARS-CoV-2 USA-WA1/2020 isolate deposited in GenBank (accession no. MN985325), the full-length genomic sequences were divided into 5 fragments (FIG. 1), synthesized de novo by Bio Basic (ON, Canada), and cloned into a high-copy-number pUC57 plasmid with designated restriction sites. A BstBI site in the S gene and an MluI site in the M gene were removed by silent mutation (FIG. 1A). These mutations were introduced to ensure that these restriction sites were unique and not present in the viral genome for the assembly of the full-length SARS-CoV-2 genome and as molecular markers to distinguish the rescued rSARS-CoV-2 clone from the natural isolate.

For the assembly of the entire viral genome in the BAC, fragment 1 was cloned into the pBeloBAC11 plasmid (NEB), linearized by PciI and HindIII digestion (FIG. 1A-FIG. 1C). By using the preassigned restriction sites in fragment 1, the other 4 fragments were assembled sequentially by using standard molecular biology methods (FIG. 1A). All the intermediate pBeloBAC11 plasmids were transformed into commercial DH10B electrocompetent E. coli cells (Thermo Fisher Scientific) using an electroporator (Bio-Rad) with the conditions of 2.5 kV, 600, and 10 F. The BAC containing the full-length SARS-CoV-2 genome, which had been analyzed by digestion using the restriction enzymes used to clone into pBeloBAC 11 (FIG. 1A), was also confirmed by deep sequencing.

Rescue of rSARS-CoV-2. Virus rescue experiments were performed as previously described (19). Briefly, confluent monolayers of Vero E6 cells (10⁶ cells/well, 6-well plates, triplicates) were transfected, using LPF2000, with 4.0-g/well of the SARS-CoV-2 BAC or an empty BAC as the internal control. After 24 hours, transfection medium was exchanged for post infection medium (DMEM supplemented with 2% [vol/vol] FBS), and cells were split and seeded into T75 flasks 48 h posttransfection. After incubation for another 72 h, tissue culture supernatants were collected, labeled as P0, and stored at 80° C. The P0 virus was used to infect fresh Vero E6 cells (10⁶ cells/well, 6-well plates, triplicates) (1 ml/well) for 48 h, and then cells were fixed and assessed for the presence of virus by immunofluorescence. After confirmation of the rescue, the P0 virus was subjected to 3 rounds of plaque purification and a new virus stock (P3) was made and titrated for further in vitro and/or in vivo experiments.

Immunofluorescence assay (IFA). Vero E6 cells (10⁶ cells/well, 6-well plate format, triplicates) were mock infected or infected (multiplicity of infection [MOI]0.01) with the natural USA-WA1/2020 isolate or rSARS-CoV-2. At 48 h post infection, cells were fixed with 10% formaldehyde solution at 4° C. overnight and permeabilized using 0.5% (vol/vol) Triton X-100 in phosphate-buffered saline (PBS) for 15 min at room temperature. Cells were incubated overnight with 1 g/ml of SARS-CoV cross-reactive N monoclonal antibody 1C7 at 4° C., washed with PBS, and stained with a fluorescein isothiocyanate (FITC)-labeled goat anti-mouse IgG (1:200). After being washed with PBS, cells were visualized and imaged under a fluorescence microscope (Olympus). Confluent monolayers of Vero E6 cells (1.2×10⁶ cells/well, 6-well format, triplicates) were mock-infected or infected (MOI of 0.01) with rSARS-CoV-2 expressing Venus or mCherry, or rSARS-CoV-2/WT. At 48 h post-infection, cells were fixed with 10% neutral buffered formalin at 4° C. for 16 h for fixation and viral inactivation, and permeabilized with phosphate-buffered saline (PBS) containing 0.5% (vol/vol) Triton X-100 for 5 min at room temperature. Cells were washed with PBS and blocked with 2.5% bovine albumin serum (BSA) in PBS for 1 h before incubation with 1 μg/ml of SARS-CoV anti-NP monoclonal antibody (MAb) 1C7 in 1% BSA in PBS for 1 h at 37° C. Cells infected with rSARS-CoV-2-Venus or -mCherry were washed with PBS and stained with either Alexa Fluor 594 goat anti-mouse IgG (Invitrogen; 1:1000) or fluorescein isothiocyanate (FITC)-conjugated goat anti-mouse IgG (Dako; 1:200), respectively. Cell nuclei were stained with 4″,6′-diamidino-2-phenylindole (DAPI, Research Organics). Representative images were captured using a fluorescence microscope (EVOS M5000 imaging system) at 20× magnification.

Plaque assay and immunostaining. Confluent monolayers of Vero E6 cells (10⁶ cells/well, 6-well plate format, triplicates) were infected with 20 PFU of SARS-CoV-2 USA-WA1/2020 or rSARS-CoV-2 for 1 h at 37° C. After viral adsorption, cells were overlaid with post infection medium containing 1% low-melting-point agar and incubated at 37° C. At 72 h post infection, cells were fixed overnight with 10% formaldehyde solution. For immunostaining, cells were permeabilized with 0.5% (vol/vol) Triton X-100 in PBS for 15 min at room temperature and immunostained using the N 1C7 monoclonal antibody (1 g/ml) and the Vectastain ABC kit (Vector Laboratories) according to the manufacturer's instructions. After being immunostained, plates were scanned and photographed using a scanner (EPSON).

Virus growth kinetics. Confluent monolayers of Vero E6 cells (10⁶ cells/well, 6-well plate format, triplicates) were infected (MOI0.01) with SARS-CoV-2 USA-WA1/2020 or rSARS-CoV-2. After 1 h of virus adsorption at 37° C., cells were washed with PBS and incubated in post infection medium at 37° C. At the indicated times after infection, viral titers in tissue culture supernatants were determined by plaque assay and immunostaining using the N monoclonal antibody 1C7, as previously described.

RNA extraction and RT-PCR. Total RNA from SARS-CoV-2 USA-WA1/2020- or rSARS-CoV-2-infected (MOI of 0.01) Vero E6 cells (10⁶ cells/well, 6-well plate format) was extracted with TRIzol reagent (Thermo Fisher Scientific) according to the manufacturer's instructions. RT-PCR amplification of the viral genome spanning nucleotides 26488 to 27784 was performed using SuperScript II reverse transcriptase (Thermo Fisher Scientific) and the Expand high-fidelity PCR system (Sigma-Aldrich). The 1,297 amplified RT-PCR products were digested with MluI (NEB). Amplified DNA products, undigested or digested with MluI, were subjected to 0.7% agarose gel analysis. Gel-purified PCR fragments were subjected to Sanger sequencing (ACGT). All primer sequences used for RT-PCR are available upon request. For certain experiments, primers specific for the viral nucleoprotein (NP) or ORF7a region; and Venus, mCherry, or Nluc were used.

Pathogenicity studies in golden Syrian hamsters. Twelve-week-old female golden Syrian hamsters were purchased from Charles River and maintained in the animal facility at Texas Biomed under specific-pathogen-free conditions. Golden Syrian hamsters were infected (1.0104 PFU) intranasally with either the rSARS-CoV-2 clone or the natural USA-WA1/2020 isolate in a final volume of 100 μl following gaseous sedation in an isoflurane chamber. After viral infection, hamsters were humanely euthanized on days 2 and 4 post infection to collect nasal turbinates and lungs.

Measurement of viral loads in nasal turbinates and lungs. Nasal turbinates and lungs from mock-, SARS-CoV-2-, and SARS-CoV-2-infected golden Syrian hamsters were homogenized in 2 ml of PBS for 20 s at 7,000 rpm using a Precellys tissue homogenizer (Bertin Instruments). Tissue homogenates were centrifuged at 12,000 g (4° C.) for 5 min, and supernatants were collected for the measurement of viral loads. Confluent monolayers of Vero E6 cells (96-plate format, 4104 cells/well, duplicate) were infected with 10-fold serial dilutions of the supernatants from the tissue homogenates. After viral adsorption for 1 h at 37° C., cells were washed 3 times with PBS before addition of fresh post infection medium containing 1% microcrystalline cellulose (Avicel; Sigma-Aldrich). Cells were further incubated at 37° C. for 24 h. Plates were then inactivated in 10% neutral buffered formalin (Thermo Fisher Scientific) for 24 h. For immunostaining, cells were washed three times with PBS and permeabilized with 0.5% Triton X-100 for 10 min at room temperature. Then, cells were blocked with 2.5% bovine serum albumin (BSA) in PBS for 1 h at 37° C., followed by incubation with 1 g/ml of the anti-N SARS-CoV monoclonal antibody 1C7 diluted in 1% BSA for 1 h at 37° C. After incubation with the primary antibody, cells were washed three times with PBS, counterstained with the Vectastain ABC kit, and developed using the DAB peroxidase substrate kit (Vector Laboratory, Inc., CA, USA) according to the manufacturer's instructions. Virus titers are indicated as PFU/ml.

Evaluation of lung pathological lesions. Macroscopic pathology scoring was evaluated using ImageJ software to determine the percentage of the total surface area of the lung (dorsal and ventral views) affected by consolidation, congestion, and atelectasis.

Statistical analysis. Data representative of three independent experiments in triplicates have been used. All data represent the mean standard deviation (SD) for each group and were analyzed with SPSS13.0 (IBM). A two-tailed Student t test was used to compare the means between two groups. P values of less than 0.05 were considered as statistically significant.

Generation of pBeloBAC11-SARS-CoV-2 encoding reporter genes. The pBeloBAC11 plasmid (NEB) containing the entire viral genome of SARS-CoV-2 has been previously described. Briefly, the entire genome sequence of SARS-CoV-2 USA/WA1/2020 (GenBank accession no. MN985325) was chemically synthesized (Bio Basic) in five fragments and cloned into pUC57 plasmids containing unique restriction sites. Silent mutations were introduced to the spike (S) and matrix (M) genes to remove BstBI and Mlul restriction sites, respectively, that were used for the assembly of the entire SARS-CoV-2 genome into the pBeloBAC11 plasmid. These nucleotide changes were also used as genetic markers to distinguish the natural USA/WA1/2020 and the recombinant SARS-CoV-2. The five fragments containing the entire SARS-CoV-2 genome were assembled into the pBeloBAC11 using standard molecular biology techniques. To remove the 7a gene and introduce the Venus, mCherry, or Nluc reporter genes, the region flanking the 7a viral gene and each individual reporter genes were amplified by extension and overlapping PCR using specific oligonucleotides in a shuttle plasmid. The modified 7a viral genes were inserted into the pBeloBAC11 plasmid containing the remaining SARS-CoV-2 viral genome using BamHI and RsrII restriction sites to generate pBeloBAC11-SARS-CoV-2-del7a/Venus, -del7a/mCherry, and -del7a/Nluc for the rescue of rSARS-CoV-2-Venus, rSARS-CoV-2-mCherry and rSARS-CoV-2-Nluc, respectively. Plasmids and pBeloBAC11 constructs were validated by Sanger sequencing (ACGT Inc).

Rescue of rSARS-CoV-2 expressing reporter genes. The rSARS-CoV-2/WT and rSARS-CoV-2 expressing reporter genes were rescued as previously described. Briefly, confluent monolayers of Vero E6 cells (1.2×10⁶ cells/well, 6-well plate format, triplicates) were transfected, using lipofectamine 2000 (LPF2000, Thermo Fisher) with 4 μg/well of pBeloBAC11-SARS-CoV-2/WT, pBeloBAC11-SARS-CoV-2-del7a/Venus, -del7a/mCherry, or -del7a/Nluc plasmids. An empty pBeloBAC11 plasmid was included as internal control. At 14 h, transfection media was replaced with post-infection media (DMEM with 2% FBS) and, 24 h later, cells were scaled up into T75 flasks. At 72 h, P0 virus-containing tissue culture supernatants were collected and stored at −80° C. Viral rescues were confirmed by infecting fresh Vero E6 cells (1.2×10⁶ cells/well, 6-well plates, triplicates) and assessing fluorescence or Nluc expression. P0 viruses were passaged three times and viral stocks were generated and titrated for in vitro experiments. Viral titers (plaque forming units per milliliter; PFU/ml) were determined by plaque assay in Vero E6 cells (1.2×10⁶ cells/well, 6-well plate format).

Protein gel electrophoresis and Western blots. Vero E6 cells (1.2×10⁶ cells/well, 6-well plate format, triplicates) were mock-infected or infected (MOI of 0.01) with rSARS-CoV-2/WT or rSARS-CoV-2 expressing Venus, mCherry, or Nluc. At 48 h post-infection, cells were lysed with 1× passive lysis buffer (Promega) and proteins were separated by denaturing electrophoresis in 12% SDS-polyacrylamide gels and transferred to a nitrocellulose membrane (Bio-Rad) with a Mini-Protean Tetra Vertical Electrophoresis Cell at 100V for 1 h at 4° C. Membranes were blocked in PBS containing 10% dried skim milk and 0.1% Tween 20 for 1 h and then incubated overnight at 4° C. with the following primary MAbs or polyclonal antibodies (PAbs). SARS-CoV NP (mouse MAb 1C7; Dr. Thomas Moran, Icahn School of Medicine at Mount Sinai), Venus (rabbit PAb sc-8334; Santa Cruz Biotech.), mCherry (rabbit PAb; Raybiotech), and Nluc (rabbit PAb; Promega). A MAb against actin (MAb AC-15; Sigma) was included as a loading control. Primary antibodies bound to the membrane were detected using horseradish peroxidase (HRP)-conjugated secondary antibodies against mouse or rabbit (GE Healthcare). Proteins were detected by chemiluminescence using SuperSignal West Femto Maximum Sensitivity Substrate (Thermo Scientific) based on the manufacturer's specifications and imaged in a ChemiDoc imaging system (Bio-Rad).

Plaque assays and immunostaining. Confluent monolayers of Vero E6 cells (1.2×10⁶ cells/well, 6-well plate format, triplicates) were infected with WT or reporter-expressing rSARS-CoV-2 for 1 h at 37° C. After viral absorption, infected cells were overlaid with agar and incubated at 37° C. for 72 h. Afterwards, cells were submerged in 10% neutral buffered formalin at 4° C. for 16 h for fixation and viral inactivation, and then the agar overlays were gently removed. To observe Venus and mCherry fluorescence expression, PBS was added to each well and plates were imaged under a fluorescence microscope (EVOS M5000 imaging system). For immunostaining, plates were permeabilized with 0.5% Triton X-100 PBS for 10 min at room temperature, blocked with 2.5% BSA PBS for 1 h at room temperature, and then incubated at 37° C. for 1 h using the anti-SARS 2 NP MAb 1C7. Plaques were developed for visualization using the Vectastain ABC kit and DAB HRP Substrate kit (Vector laboratories), in accordance to the manufacturer's recommendations.

Viral growth kinetics and titrations. Vero E6 cells (1.2×10⁶ cells/well, 6-well plate format, triplicates) were infected (MOI of 0.01) with rSARS-CoV-2/WT or rSARS-CoV-2 expressing Venus, mCherry, or Nluc. After viral adsorption for 1 h at 37° C., cells were washed with PBS, provided with fresh post-infection media, and then placed in a 37° C. incubator with 5% CO2 atmosphere. At the indicated times post-infection (12, 24, 48, 72, and 96 h), cells were imaged for Venus or mCherry expression under a fluorescence microscope (EVOS M5000 imaging system). Viral titers in the tissue culture supernatants at each time point were determined by titration and immunostaining, as previously described, using the anti-SARS-CoV NP MAb 1C7. Nluc expression in tissue culture supernatants was quantified using Nano-Glo luciferase substrate (Promega) following the manufacturer's recommendations. Mean values and standard deviation (SD) were determined using GraphPad Prism software (version 8.2).

Reporter-based micro-neutralization assay for the identification of antivirals. Vero E6 cells (96-well plate format, 4×104 cells/well, quadruplicates) were infected with ˜100-200 PFU of rSARS-CoV-2/WT or rSARS-CoV-2 expressing Venus, mCherry, or Nluc for 1 h at 37° C. After viral adsorption, cells were washed and incubated in 100 μL of infection media (DMEM with 2% FBS) containing 3-fold serial dilutions (starting concentration of 50 μM) of Remdesivir, or 0.1% DMSO vehicle control, and 1% avicel (Sigma-Aldrich). Cells infected with rSARS-CoV-2/WT or rSARS-CoV-2 expressing fluorescent Venus or mCherry were incubated at 37° C. for 24 h, while cells infected with rSARS-CoV-2 expressing Nluc were incubated at 37° C. for 48 h. For rSARS-CoV-2/WT and rSARS-CoV-2 expressing fluorescent Venus and mCherry, cells were submerged in 10% neutral buffered formalin at 4° C. for 16 h for fixation and viral inactivation. Cells were washed with 100 μl/well of PBS three times, permeabilized with 100 μl/well of 0.5% Triton X-100 in PBS at room temperature for 15 min and blocked with 100 μl/well of 2.5% BSA in PBS at 37° C. for 1 h. Next, cells were staining with the anti-NP MAb 1C7 (1 μg/mL) in 1% BSA PBS at 37° C. for 1 h. After incubation with the primary MAb, cells were washed with PBS three times, and a secondary fluorescein isothiocynate (FITC)-conjugated goat anti-mouse IgG (Dako; 1:200) in 1% BSA were added to cells for 1 h at 37° C. Cell nuclei were stained with 4″,6′-diamidino-2-phenylindole (DAPI, Research Organics). Viral infections were determined using fluorescent images of each well and quantified using a cell image analysis software, Cell Profiler (Broad Institute). In the case of cells infected with rSARS-CoV-2 expressing Nluc, tissue culture supernatants were collected at 48 h post-infection and Nluc expression was measured using a luciferase assay and a Synergy LX microplate reader (BioTek). Individual wells from three independent experiments conducted in quadruplicates were used to calculate the average and standard deviation (SD) of viral inhibition using Microsoft Excel software. Non-linear regression curves and the half maximal effective concentration (EC50) of Remdesivir was determined using GraphPad Prism software (version 8.2).

Reporter-based microneutralization assay for the identification of NAbs. To test the neutralizing activity of 1212C2, a human MAb recently described to neutralize SARS-CoV-2 (23), confluent monolayers of Vero E6 cells (96-plate format, 4×104 cells/well, quadruplicates) were infected with -100-200 PFU of rSARS-CoV-2/WT or rSARS-CoV-2 expressing Venus, mCherry, or Nluc for 1 h at 37° C. After viral adsorption, cells were washed and incubated with 100 μL of infection media (DMEM 2% FBS) containing 3-fold serial dilutions (starting concentration of 500 ng) of 1212C2 or PBS, and 1% avicel (Sigma-Aldrich). Infected cells were incubated at 37° C. for 24 h for rSARS-CoV-2/WT, or rSARS-CoV expressing Venus or mCherry, and 48 h for rSARS-CoV-2 expressing Nluc. After viral infections, cells infected with rSARS-CoV-2/WT and rSARS-CoV-2 expressing fluorescent Venus and mCherry were submerged in 10% neutral buffered formalin at 4° C. for 16 h for fixation and viral inactivation. Cells were washed with 100 μl/well of PBS three times, permeabilized with 100 μl/well of 0.5% Triton X-100 in PBS at room temperature for 15 min. Then, cells were blocked with 100 μl/well of 2.5% BSA in PBS at 37° C. for 1 h. Cells were next incubated with the anti-NP MAb 1C7 (1 g/ml) in 1% BSA PBS at 37° C. for 1 h. Cells were next washed three times with PBS and incubated with a secondary fluorescein isothiocynate (FITC)-conjugated goat anti-mouse IgG (Dako; 1:200) in 1% BSA for 1 h at 37° C. Cell nuclei were stained with 4″,6′-diamidino-2-phenylindole (DAPI, Research Organics). Viral infections were determined using fluorescent images of each well and quantified using a cell image analysis software, Cell Profiler (Broad Institute). In the case of cells infected with rSARS-CoV-2 expressing Nluc, tissue culture supernatants were collected at 48 h post-infection and Nluc expression was measured using a luciferase assay and a Synergy LX microplate reader (BioTek). Individual wells from three independent experiments conducted in quadruplicates were used to calculate the average and standard deviation (SD) of viral inhibition using Microsoft Excel software. Non-linear regression curves and the half maximal neutralizing concentration (NT50) of 1212C2 was determined using GraphPad Prism software (version 8.2).

Genetic stability. Vero E6 cells (1.2×10⁶ cells/well, 6-well plate format, triplicates) were infected (MOI of 0.01) with rSARS-CoV-2-Venus or -mCherry P3 stocks and after 1 h viral adsorption, virus inoculum was replaced with infectious media (DMEM 2% FBS). The cells were incubated at 37° C. with 5% C02 until 70% cytopathic effect (CPE) was observed. Then, tissue culture supernatants were collected and diluted 100-fold in infectious media and used to infect fresh Vero E6 cells (1.2×10⁶ cells/well, 6-well format, triplicates) for two additional passages (P5). Venus- and mCherry-expressing plaques (˜50 counted plaques per viral passage) were evaluated by immunostaining and fluorescent protein expression. Viral plaques were imaged under a fluorescence microscope (EVOS M5000 imaging system) under 4× magnification.

Generation of pBeloBAC11-SARS-CoV-2 encoding fluorescent proteins (FP). The pBeloBAC11 plasmid (NEB) containing the entire viral genome of SARS-CoV-2 USA/WA1/2020 (WA-1) isolate (accession no. MN985325) has been previously described. The rSARS-CoV-2 expressing Venus or mCherry from the locus of the viral N protein using the PTV-1 2A autocleavage sequence were generated as previously described. The rSARS-CoV-2 containing mutations K417N, E484K, and N501Y present in the receptor binding domain (RBD) within the spike (S) gene of the South African (SA) B.1.351 (beta, f) VoC and expressing mCherry was generated using standard molecular biology techniques. Plasmids containing the full-length genome of the different rSARS-CoV-2 were analyzed by digestion using specific restriction enzymes and validated by deep sequencing. Oligonucleotides for cloning the Venus or mCherry FP, or K417N, E484K, and N501Y mutations, are available upon request.

Generation of rSARS-CoV-2 expressing FP. Wild-type (WT, WA-1), Venus (Venus WA-1), and mCherry (mCherry WA-1) reporter-expressing rSARS-CoV-2, as well as rSARS-CoV-2 encoding the SA B.1.351 (beta, f) mutations K417N, E484K, and N501Y in the S RBD expressing mCherry (mCherry SA) were rescued as previously described. Briefly, confluent monolayers of Vero E6 cells (1.2×10⁶ cells/well, 6-well plate format, triplicates) were transfected with 4 μg/well of pBeloBAC11-SARS-CoV-2 (WA-1), -2A/Venus, -2A/mCherry, or -2A/mCherry-SA-RBD plasmids using Lipofectamine 2000 (Thermo Fisher). After 24 h post-transfection, media was exchanged with post-infection (pi) media (DMEM containing 2% FBS), and 24 h later cells were scaled up to T75 flasks and incubated for 72 h at 37° C. Viral rescues were first confirmed under a brightfield microscope by assessing cytopathic effect (CPE) before supernatants were collected, aliquoted, and stored at −80° C. To confirm the rescue of rSARS-CoV-2, Vero E6 cells (1.2×10⁶ cells/well, 6-well plates, triplicates) were infected with virus-containing tissue culture supernatants and incubated at 37° C. in a 5% C02 incubator for 48 h. Viruses were detected by fluorescence or immunostaining with a SARS-CoV N protein cross reactive mouse (m)MAb (1C7C7). Plaque assays were used to determine viral titers (plaque forming units, PFU)/ml). Viral stocks were generated by infecting fresh monolayers of Vero E6 cells at low multiplicity of infection (MOI, 0.0001) for 72 h before aliquoted and stored at −80° C.

Sequencing. To confirm the identity of the rescued rSARS-CoV-2 mCherry SA, total RNA from infected (MOI 0.01) Vero E6 cells (1.2×10⁶ cells/well, 6-well format, triplicates) was extracted using TRIzol reagent (Thermo Fisher Scientific), and used in RT-PCR reactions to amplify ta fragment of 1,174 bp around the RBD of the S gene. RT-PCR was done using SuperScript II reverse transcriptase (Thermo Fisher Scientific) and Expand high-fidelity PCR system (Sigma-Aldrich). RT-PCR products were purified on 0.7% agarose gel and subjected to Sanger sequencing (ACGT). All primer sequences are available upon request.

Immunofluorescence assays. Confluent monolayers of Vero E6 cells (1.2×10⁶ cells/well, 6-well format, triplicates) were mock-infected or infected (MOI 0.01) with WT, Venus- or mCherry-expressing rSARS-CoV-2 WA-1 (WA-1, Venus WA-1, or mCherry WA-1, respectively) or rSARS-CoV-2 mCherry SA. At 48 hours post-infection (hpi), cells were submerged in 10% neutral buffered formalin at 4° C. overnight for fixation and viral inactivation, and then permeabilized with 0.5% Triton X-100 phosphate-buffered saline (PBS) at room temperature for 10 min. Thereafter, cells were washed with PBS before blocking with 2.5% bovine albumin serum (BSA) PBS for 1 h. Cells were then incubated with 1 μg/ml of SARS-CoV anti-N mMAb 1C7C7 in 1% BSA at 37° C. for 1 h. Reporter-expressing rSARS-CoV-2 were detected directly by epifluorescence and using either Alexa Fluor 594 goat anti-mouse IgG (Invitrogen; 1:1,000) or fluorescein isothiocynate (FITC)-conjugated goat anti-mouse IgG (Dako; 1:200), depending on whether the viruses express Venus or mCherry, respectively. Cell nuclei were detected with 4′, 6′-diamidino-2-phenylindole (DAPI, Research Organics). An EVOS M5000 imaging system was used to acquire representative images (10× magnification).

Viral growth kinetics. Vero E6 cells (1.2×10⁶ cells/well, 6-well plate format, triplicates) were infected (MOI 0.01) at 37° C. for 1 h. After viral adsorption, cells were washed with PBS and incubated at 37° C. in p.i. media. At 24, 48, 72, and 96 hpi, fluorescence-positive cells were imaged with an EVOS M5000 fluorescence microscope for rSARS-CoV-2 expressing Venus or mCherry FP, and viral titers in the tissue culture supernatants were determined by plaque assay and immunostaining using the anti-SARS-CoV N mMAb 1C7C7. Mean values and standard deviation (SD) were calculated with Microsoft Excel software.

Plaque assays and immunostaining. Confluent monolayers of Vero E6 cells (2×10¹ cells/well, 24-well plate format, triplicates) were infected with WT or reporter-expressing rSARS-CoV-2 for 1 h before being overlaid with p.i. media containing 1% agar (Oxoid) and incubated at 37° C. in a 5% CO2 incubator. After 72 h, cells were fixed in 10% neutral buffered formalin overnight at 4° C. Next, overlays were removed, PBS was added to each well, and fluorescent plaques were detected and quantified using a ChemiDoc MP imaging system (Bio-Rad). Cells were then permeabilized with 0.5% Triton X-100 in PBS for 5 min, blocked with 2.5% BSA in PBS for 1 h, and incubated with the SARS-CoV N mMAb 1C7C7, and plaques detected using a Vectastain ABC kit and DAB HRP Substrate kit (Vector laboratories) following the manufacturers' instructions.

A bifluorescent-based neutralization assay. The hMAbs used in this study were generated and purified as described. CB6, REGN10987 and REGN10933 hMAbs were included as controls. To test the neutralizing activity of hMAbs, confluent monolayers of Vero E6 cells (4×104 cells/well, 96-plate well format, quadruplicates) were infected (MOI of 0.01 or 0.1) with the indicated rSARS-CoV-2 for 1 h at 37° C. After viral absorption, p.i. media containing 3-fold dilutions of the indicated hMAbs (starting concentration of 500 ng/well) were added to the cells and incubated at 37° C. for 48 h. Cells were then fixed in 10% neutral buffered formalin overnight and washed with PBS, before fluorescence signal was measured and quantified using a Synergy LX microplate reader and Gen5 data analysis software (Bio-Tek). The mean and SD of viral infections were calculated from individual wells of three independent experiments conducted in quadruplicates with Microsoft Excel software. Non-linear regression curves and NT50 values were determined using GraphPad Prism Software (San Diego, CA, USA, Version 8.2.1). Representative images were captured with an EVOS M5000 Imaging system (Thermofisher) at 10× magnification.

Mouse experiments. All animal protocols were approved by Texas Biomed IACUC (1718MU). Six-to-eight-week-old female K18 human angiotensin converting enzyme 2 (hACE2) transgenic mice were purchased from The Jackson Laboratory and maintained in the Animal Biosafety Laboratory level 3 (ABSL-3) at Texas Biomedical Research Institute. All mouse procedures were approved by Texas Biomedical Research Institute IACUC. To assess the in vivo efficacy of hMAbs, K18 hACE2 transgenic mice (n=5/group) were anesthetized with isoflurane and injected intraperitoneally (i.p.) with hMAbs IgG isotype control, 1212C2 or 1213H7 (25 mg/kg) using a 1 ml syringe 23-25 gauge ⅝-inch needle 24 h prior to challenge with rSARS-CoV-2. For viral challenges, mice were anesthetized and inoculated intranasally (i.n.) with 104 plaque forming units (PFU) of the indicated rSARS-CoV-2 and monitored daily for morbidity as determined by changes in body weight, and survival. Mice that lost greater than 25% of their initial weight were considered to have reached their experimental endpoint and were humanely euthanized. In parallel, K18 hACE2 transgenic mice (n=3/group) were treated (i.p.) with 1212C2 or 1213H7 hMAbs and challenged i.n. with 104 PFU of the indicated rSARS-CoV-2 for viral titer determination. Viral titers in the lungs of infected mice at days 2 and 4 p.i. were determined by plaque assay. In vivo fluorescence imaging of mouse lungs was conducted using an Ami HT in vivo imaging system, IVIS (Spectral Instruments). Mice were euthanized with a lethal dose of Fatal-Plus solution and lungs were surgically extracted and washed in PBS before imaging in the Ami HT. Images were analyzed with Aura software to determine radiance with the region of interest (ROI), and fluorescence signal was normalized to background signal of lungs from mock-infected mice. Bright field images of lungs were captured using an iPhone X camera. After imaging, lungs were homogenized using a Precellys tissue homogenizer (Bertin Instruments) in 1 ml of PBS and centrifuged at 21,500×g for 10 min to pellet cell debris. Clarified supernatants were collected and used to determine viral titers by plaque assay. Macroscopic pathological scoring was determined from the percent of total surface area affected by congestion, consolidation, and atelectasis of excised lungs, using NIH ImageJ software as previously described.

Reverse-genetics system. The BAC harboring the entire viral genome of SARS-CoV-2 USA-WA1/2020 (GenBank accession no. MN985325) was constructed as described previously. Deletion of individual accessory ORF proteins was achieved in viral fragment 1 by using inverse PCR and primer pairs containing a BsaI type IIS restriction endonuclease site. All the primer sequences are available upon request. Fragments, including those with individual deletions of accessory ORF proteins, were reassembled into the BAC using BamHI and RsrII restriction endonucleases.

Rescue of ΔORF rSARS-CoV-2s. Virus rescues were performed as previously described. Briefly, confluent monolayers of Vero E6 cells (10⁶ cells/well, 6-well plates, triplicates) were transfected with 4.0 mg/well of the SARS-CoV-2 BAC using Lipofectamine 2000. After 24 h, transfection medium was exchanged for p.i. medium (DMEM supplemented with 2% vol/vol of FBS), and cells were split and seeded into T75 flasks 48 h post-transfection. After incubation for another 72 h, tissue culture supernatants were collected, labeled as P0, and stored at 280° C. After viral titration of the supernatant, the P0 virus was used to infect fresh Vero E6 cells at a multiplicity of infection (MOI) of 0.0001 to make new viral stocks. Tissue culture supernatants were collected 72 h p.i., aliquoted, labeled as P1, and stored at 280° C. for future use. To sequence the viral stocks, viral RNA was purified using the Direct-zol RNA MiniPrep Plus (Zymo) kit according to the manufacturer's instructions. Whole-genome amplification and sequencing were performed as previously described.

Immunofluorescence assay (IFA). Vero E6 cells (10⁵ cells/well, 24-well plate format, triplicates) were mock infected or infected (MOI of 3) with the WT or a ΔORF rSARS-CoV-2. At 24 h p.i., cells were fixed with 10% formaldehyde solution at 4° C. overnight and permeabilized using 0.5% (vol/vol) Triton X-100 in PBS for 15 min at room temperature. Cells were incubated overnight with 1 mg/ml of a SARS-CoV N protein cross-reactive polyclonal antibody at 4° C. and a SARS-CoV-2 spike (S) cross-reactive monoclonal anti-body (3B4), washed with PBS, and stained with a fluorescein isothiocyanate (FITC)-labeled goat anti-mouse IgG (1:200) and rhodamine-labeled goat anti-rabbit IgG (1:200). Nuclei were visualized by DAPI (49,6-diamidino-2-phenylindole) staining. After being washed with PBS, cells were visualized and imaged with an EVOS microscope (ThermoFisher Scientific).

Plaque assay and immunostaining. Confluent monolayers of Vero E6 cells (10⁶ cells/well, 6-well plate format, triplicates) were infected with serially diluted viruses for 1 h at 37° C. After viral adsorption, cells were overlaid with p.i. medium containing 1% low-melting-point agar and incubated at 37° C. At 72 h p.i., cells were fixed overnight with 10% formaldehyde solution. For immunostaining, cells were permeabilized with 0.5% (vol/vol) Triton X-100 in PBS for 15 min at room temperature and immunostained using the SARS-CoV N protein cross-reactive monoclonal antibody 1C7C7 (1 mg/ml) and the Vectastain ABC kit (Vector Laboratories) by following the manufacturers' instructions. After the immunostaining, plates were visualized on a ChemiDoc imager (Bio-Rad). Viral plaque diameters were measured with a standard ruler in centimeters.

Virus growth kinetics. Confluent monolayers of Vero E6 cells (10⁶ cells/well, 6-well plate format, triplicates) were infected (MOI of 0.01) with the WT or a ΔORF rSARS-CoV-2. After 1 h of virus adsorption at 37° C., cells were washed with PBS and incubated in p.i. medium at 37° C. At the indicated times after infection, viral titers in tissue culture supernatants were determined by plaque assay and immunostaining using the SARS-CoV N protein cross-reactive monoclonal antibody 1C7C7.

RT-PCR. Total RNA from Vero E6 cells (10⁶ cells/well, 6-well plate format) infected (MOI of 0.01) with the WT or a ΔORF rSARS-CoV-2 was extracted with TRIzol reagent (Thermo Fisher Scientific) according to the manufacturer's instructions. Reverse transcription-PCR (RT-PCR) amplification was performed using SuperScript II reverse transcriptase (Thermo Fisher Scientific) and an expanded high-fidelity PCR system (Sigma-Aldrich). The amplified DNA products were subjected to 0.7% agarose gel analysis, and the gel-purified PCR fragments were subjected to Sanger sequencing (ACGT). All primer sequences used for RT-PCR are available upon request. Methods for Illumina library preparation, sequencing, and analysis of recombinant viruses were followed as known in the art.

Mice. Four- to 8-week-old specific-pathogen-free female B6.Cg-Tg(K18-ACE2)2Prlmn/J (stock no. 034860) K18 hACE2 transgenic mice were purchased from The Jackson Laboratory (Bar Harbor, ME). Significant sex-dependent differences were not observed in morbidity, mortality, or viral titers. K18 hACE2 transgenic mice were maintained in microisolator cages at ABSL3, provided sterile water and chow ad libitum, and acclimatized for 1 week prior to experimental manipulation. For morbidity and mortality studies, a total of 20 females were used (n=4/group), whereas a total of 40 female mice (n=4 per group) were used to determine viral titers at 2 and 4 days p.i. For the mock-infected control groups in the morbidity and mortality studies, 2 female K18 hACE2 transgenic mice were used and a total of 4 (n=2 per time point) were used as mock-infected controls for gross pathology analysis.

Mouse infection and sample processing. K18 hACE2 transgenic mice were either mock (PBS) infected or infected intranasally (i.n.) with 105 PFU of the WT or a ΔORF rSARS-CoV-2 in a final volume of 50 ml following isoflurane sedation. After viral infection, mice were monitored daily for 14 days for morbidity (body weight loss) and mortality (survival). Mice showing 25% loss of their initial body weight were defined as reaching the experimental endpoint and humanely euthanized. For viral titers and gross pathology analysis, K18 hACE2 transgenic mice were infected as described above but euthanized at 2 or 4 days p.i. Nasal turbinates and lung tissues were harvested and homogenized in 1 ml of PBS using a Precellys tissue homogenizer (Bertin Instruments) for viral titrations. Tissue homogenates were centrifuged at 21,500 g for 5 min, and supernatants were collected for determination of viral titer.

Multiplex cytokine assay. Multiple cytokines and chemokines (IFN-α, IFN-g, IL-10, IL-17A, IL-6, MCP-1 or CCL2, RANTES or CCL5, TNF-α) were measured using a custom 8-plex panel mouse ProcartaPlex assay (ThermoFisher Scientific; catalog no. PPX-08-MXGZGFX, lot 279751-000) by following the manufacturer's instructions. An immunoassay was performed in the ABSL3 laboratory, and samples were decontaminated by an overnight incubation in 1% formaldehyde solution before readout on a Luminex 100/200 system running on xPONENT v4.2 with the following parameters: gate, 5,000 to 25,000; 50 ml of sample volume; 50 to 100 events per bead; sample timeout, 120 s; and low photomultiplier tube (PMT) (LMX100/200 default). Acquired data were analyzed using ProcartaPlex Analysis software v1.0. Statistical analysis. Statistical analysis was performed using GraphPad Prism 8.3. For multiple comparisons, a 2-way analysis of variance (ANOVA) with multiple comparisons was performed.

Rescue of double deletion rSARS-CoV-2. Virus rescues were performed as previously described. Briefly, confluent monolayers of Vero E6 cells (10⁶ cells/well, 6-well plates, triplicates) were transfected with 4.0 μg/well of SARS-CoV-2 BAC using Lipofectamine 2000. After 24 h, transfection media was exchanged for post-infection media (DMEM supplemented with 2% (v/v) FBS), and cells were split and seeded into T75 flasks at 72 h post-transfection. After incubation for another 72 h, tissue culture supernatants were collected, labeled as P0 and stored at −80° C. After viral titration of the supernatant, the P0 virus was used to infect fresh Vero E6 cells at MOI 0.0001 for 72 h to produce P1 stocks. P1 stocks were aliquoted, titrated and stored at −80° C. for future use. For generation of stock for in vivo studies, a new P1 stock was generated, concentrated with polyglycol ethylene (PEG) following manufacturer's protocol, titrated, and stored at −80° C.

Plaque reduction microneutralization assay. SARS-CoV-2-specific NAbs were determined using an endpoint dilution plaque reduction microneutralization (PRMNT) assay. All the serum samples were diluted starting from 1:50 and then subjected to 2-folds serial dilutions were mixed with an equal volume of DMEM containing approximately 100 PFU/well SARS-CoV-2 USA-WA1/2020, United Kingdom variant B.1.1.7, Brazil/Japan variant P.1, South Africa variant B.1.351, or California variant B.1.429 in a 96-well plate, and the plate was incubated at 37° C. for 1 h with constant rotation. The mixtures were then transferred to confluent Vero E6 cells in 96-well plates. After incubation at 37° C. for 1h, the virus-serum mixtures were removed and a fresh DMEM with 2% FBS was added. At 24 h post-infection, cells were fixed in 10% formalin solution and immunostained with a SARS-CoV cross-reactive nucleocapsid (N) protein monoclonal antibody (mAb, 1C7C7). Virus neutralization was quantified using ELISPOT, and the percentage of infectivity calculated using sigmoidal dose response curve. Mock-infected cells (no virus) and cells infected with SARS-CoV-2 in the absence of serum were included as internal controls. NT50 was calculated for each serum sample. Data were expressed as mean±SD.

Multiplex cytokine assay. Multiple cytokines and chemokines (IFN-α, IFN-γ, IL-6, IL-10, TNF-α, IL-17A, MCP-1 or CCL2, and RANTES or CCL5,) were measured using a custom 8-plex panel mouse ProcartaPlex assay (ThermoFisher Scientific, cat. number: PPX-08-MXGZGFX, lot: 283828-000), following the manufacturer's instructions. The assay was performed in the ABSL-3 laboratory and samples were decontaminated by an overnight incubation in 1% formaldehyde solution before readout on a Luminex 100/200 System running on Xponent v4.2 with the following parameters: gate 5,000-25,000, 50 μl of sample volume, 50 events per bead, sample timeout 120 s, low PMT (LMX100/200: Default). Acquired data were analyzed using ProcartaPlex Analysis Software v1.0.

In vivo imaging. For in vivo bioluminescence imaging, mice were anesthetized with isoflurane, injected retro-orbitally with 100 μl of Nano-Glo luciferase substrate (Promega), and immediately imaged. The bioluminescence data acquisition and analysis were performed using the Aura program (Spectral Imaging Systems). Flux measurements were acquired from the region of interest.

While these embodiments have been described with emphasis on the embodiments, it should be understood that within the scope of the appended claims, the embodiments might be practiced other than as specifically described herein. Although the embodiments have been shown in only limited forms, it should be apparent to those skilled in the art that it is not so limited but susceptible to various changes without departing from the scope of the disclosure. Accordingly, it is intended to embrace all such alternatives, modifications, and variations as fall within the spirit and broad scope of the appended claims.

What is claimed is:

1. A bacterial artificial chromosome-based construct comprising a replication-competent recombinant severe acute respiratory syndrome coronavirus-2 (SARS-CoV-2) genome, wherein the SARS-CoV-2 genome contains a deletion of a group-specific open reading frame and a reporter gene adapted to report transcription of the recombinant SARS-CoV-2 genome.

2. The bacterial artificial chromosome-based construct of claim 1, wherein the group-specific open reading frame is one or more of Spike (S), Membrane (M), Envelope (E), ORF3a, ORF6, ORF7a, ORF7B, ORF8, and ORF10.

3. The bacterial artificial chromosome-based construct of claim 1, wherein the reporter gene encodes one or more of a fluorescent protein.

4. The bacterial artificial chromosome-based construct of claim 1, wherein the reporter gene encodes one or more of a fluorescent protein and a luciferase.

5. A method of stimulating an immune response against SARS-CoV-2 in a subject comprising administering an immunogenic composition containing the bacterial artificial chromosome-based construct of claim 1.

6. A reverse genetics system for screening and identifying an anti-SARS-CoV-2 agent, the system comprising a bacterial artificial chromosome-based vector containing a replication-competent recombinant SARS-CoV-2 genome and a reporter gene adapted to report transcription of the recombinant SARS-CoV-2 genome.

7. The reverse genetics system of claim 6, wherein the reporter gene encodes one or more of a fluorescent protein.

8. The reverse genetics system of claim 6, wherein the reporter gene encodes one or more of a fluorescent protein and a luciferase.

9. The reverse genetics system of claim 6, wherein the anti-SARS-CoV-2 agent is a neutralizing antibody.

10. A bacterial artificial chromosome-based vector comprising a replication-competent recombinant SARS-CoV-2 genome, wherein the SARS-CoV-2 genome contains a mutation in the gene encoding for a spike protein and a reporter gene adapted to report transcription of the recombinant SARS-CoV-2 genome.

11. The bacterial artificial chromosome-based vector of claim 10, wherein the mutation is a Bristol deletion.

12. The bacterial artificial chromosome-based vector of claim 10, wherein the mutation is a Furin deletion.

13. The bacterial artificial chromosome-based vector of claim 10, wherein the reporter gene encodes one or more of a fluorescent protein.

14. The bacterial artificial chromosome-based vector of claim 10, wherein the reporter gene encodes one or more of a fluorescent protein and a luciferase.

15. A method of stimulating an immune response against SARS-CoV-2 in a subject comprising administering an immunogenic composition containing the bacterial artificial chromosome-based vector of claim 10.
